SEQUENCE LISTING (45 sequences)

SEQ ID NO: 1 - DNA, 39 bp, synthetic construct
  misc_feature 1..16: note = DNA
  misc_feature 17..36: note = RNA
  misc_feature 37..39: note = DNA
  source 1..39: mol_type = other DNA
atatatattattattacatagatcgcgtacaatacgtcg

SEQ ID NO: 2 - DNA, 57 bp, synthetic construct
  source 1..57: mol_type = other DNA
acgtccacctttttccacgatttttggtggacgtcgacgtattgtacgcgatctatg

SEQ ID NO: 3 - DNA, 78 bp, synthetic construct
  misc_feature 1..16: note = DNA
  misc_feature 17..30: note = RNA
  misc_feature 31..78: note = DNA
  source 1..78: mol_type = other DNA
atatatattattattgtcgcgtacaatacgtcgacgtccacctccacgatttggtggacgtcgacgtattgtacgcga

SEQ ID NO: 4 - DNA, 60 bp, synthetic construct
  misc_feature 1..8: note = DNA
  misc_feature 9..22: note = RNA
  misc_feature 23..60: note = DNA
  source 1..60: mol_type = other DNA
atatatagtcgcgtacaatacggtccacctccacgatttggtggaccgtattgtacgcga

SEQ ID NO: 5 - DNA, 72 bp, synthetic construct
  misc_feature 1..8: note = DNA
  misc_feature 9..22: note = RNA
  misc_feature 23..72: note = DNA
  source 1..72: mol_type = other DNA
atatatattcgcgtacaatacgtccacgtccaggtccacgatttcctggacgtggacgtattgtacgcgatc

SEQ ID NO: 6 - DNA, 62 bp, synthetic construct
  misc_feature 1..14: note = RNA
  misc_feature 15..62: note = DNA
  source 1..62: mol_type = other DNA
tcgcgtacaatacgtccacgtccaggtccacgatttcctggacgtggacgtattgtacgcga

SEQ ID NO: 7 - DNA, 67 bp, synthetic construct
  source 1..67: mol_type = other DNA
atatatattattgagatcgcatggcatagcatcacacgatgtacgttcatagatcgcgtacaatacg

SEQ ID NO: 8 - DNA, 34 bp, synthetic construct
  misc_feature 1..7: note = DNA
  misc_feature 8..15: note = RNA
  misc_feature 16..34: note = DNA
  source 1..34: mol_type = other DNA
atatttaatattattagatcgcatggcatagcat

SEQ ID NO: 9 - DNA, 35 bp, synthetic construct
  source 1..35: mol_type = other DNA
cacacgatgtacgttcatagatcgcgtacaatacg

SEQ ID NO: 10 - DNA, 21 bp, synthetic construct
  source 1..21: mol_type = other DNA
tacatcgtgtgatgctatgcc

SEQ ID NO: 11 - DNA, 15 bp, synthetic construct
  source 1..15: mol_type = other DNA
  modified_base 1: mod_base = OTHER; note = Locked Nucleic Acid (LNA) base
tccacgtccaggtcc

SEQ ID NO: 12 - DNA, 21 bp, synthetic construct
  source 1..21: mol_type = other DNA
gagatcgcatggcatagcatc

SEQ ID NO: 13 - DNA, 54 bp, synthetic construct
  source 1..54: mol_type = other DNA
  misc_binding 1..14: bound_moiety = P5-G7 index primer
  misc_feature 34..54: note = Region anneals to the extension product template
tcgtcggcagcgtcagatgtgtataagagacaggagatcgcatggcatagcatc

SEQ ID NO: 14 - DNA, 49 bp, synthetic construct
  source 1..49: mol_type = other DNA
  misc_feature 35..49: note = Region anneals to the extension product template
gtctcgtgggctcggagatgtgtataagagacagtccacgtccaggtcc

SEQ ID NO: 15 - DNA, 47 bp, synthetic construct
  source 1..47: mol_type = other DNA
  misc_feature 1..24: note = Illumina P7 adapter regions
  misc_feature 33..47: note = Region anneals to the products from the library prep first step
caagcagaagacggcatacgagatgccttaacgtctcgtgggctcgg

SEQ ID NO: 16 - DNA, 51 bp, synthetic construct
  source 1..51: mol_type = other DNA
  misc_feature 1..29: note = Illumina P5 adapter regions
  misc_feature 38..51: note = Region anneals to the products from the library prep first step
aatgatacggcgaccaccgagatctacactaggagcttcgtcggcagcgtc

SEQ ID NO: 17 - DNA, 20 bp, synthetic construct
  source 1..20: mol_type = other DNA
aatgatacggcgaccaccga

SEQ ID NO: 18 - DNA, 21 bp, synthetic construct
  source 1..21: mol_type = other DNA
caagcagaagacggcatacga

SEQ ID NO: 19 - DNA, 96 bp, synthetic construct
  misc_feature 1..16: note = DNA
  misc_feature 17..36: note = RNA
  misc_feature 37..96: note = DNA
  source 1..96: mol_type = other DNA
atatatattattattacatagatcgcgtacaatacgtcgacgtccacctttttccacgatttttggtggacgtcgacgtattgtacgcgatctatg

SEQ ID NO: 20 - DNA, 78 bp, synthetic construct; identical to SEQ ID NO: 3
  misc_feature 1..16: note = DNA
  misc_feature 17..30: note = RNA
  misc_feature 31..78: note = DNA
  source 1..78: mol_type = other DNA

SEQ ID NO: 21 - DNA, 60 bp, synthetic construct; identical to SEQ ID NO: 4
  misc_feature 1..8: note = DNA
  misc_feature 9..22: note = RNA
  misc_feature 23..60: note = DNA
  source 1..60: mol_type = other DNA

SEQ ID NO: 22 - DNA, 72 bp, synthetic construct; identical to SEQ ID NO: 5
  misc_feature 1..8: note = DNA
  misc_feature 9..22: note = RNA
  misc_feature 23..72: note = DNA
  source 1..72: mol_type = other DNA

SEQ ID NO: 23 - DNA, 62 bp, synthetic construct; identical to SEQ ID NO: 6
  misc_feature 1..14: note = RNA
  misc_feature 15..62: note = DNA
  source 1..62: mol_type = other DNA

SEQ ID NO: 24 - DNA, 64 bp, synthetic construct
  source 1..64: mol_type = other DNA
  misc_feature 7..23: note = T7p
  misc_feature 51..64: note = Capture region
tatatataatacgactcactatagggtccacgtccaggtccgacgattttcgtattgtacgcga

SEQ ID NO: 25 - DNA, 64 bp, synthetic construct
  source 1..64: mol_type = other DNA
  misc_feature 1..14: note = Capture region
  misc_feature 24..38: note = CO DS primer 1 landing site
  misc_feature 42..58: note = T7p
tcgcgtacaatacgaaaatcgtcggacctggacgtggaccctatagtgagtcgtattatatata

SEQ ID NO: 26 - DNA, 38 bp, synthetic construct
  source 1..38: mol_type = other DNA
  misc_feature 25..38: note = Capture region
tccacgtccaggtccgacgattttcgtattgtacgcga

SEQ ID NO: 27 - DNA, 37 bp, synthetic construct
  source 1..37: mol_type = other DNA
  misc_feature 24..37: note = Capture region
ccacgtccaggtccgacgattttcgtattgtacgcga

SEQ ID NO: 28 - DNA, 38 bp, synthetic construct
  source 1..38: mol_type = other DNA
  misc_feature 1..15: note = Capture region
  misc_feature 24..38: note = CO DS primer 1 landing site
tcgcgtacaatacgaaaatcgtcggacctggacgtgga

SEQ ID NO: 29 - DNA, 27 bp, synthetic construct
  source 1..27: mol_type = other DNA
  misc_feature 7..23: note = T7p
tatatataatacgactcactatagggt

SEQ ID NO: 30 - DNA, 26 bp, synthetic construct
  source 1..26: mol_type = other DNA
  misc_feature 4..20: note = T7p
ccctatagtgagtcgtattatatata

SEQ ID NO: 31 - DNA, 15 bp, synthetic construct
  source 1..15: mol_type = other DNA
tccacgtccaggtcc

SEQ ID NO: 32 - DNA, 17 bp, synthetic construct
  source 1..17: mol_type = other DNA
agatcgcatggcatagc

SEQ ID NO: 33 - DNA, 35 bp, synthetic construct; identical to SEQ ID NO: 9
  source 1..35: mol_type = other DNA
  misc_feature 22..35: note = Capture region

SEQ ID NO: 34 - DNA, 34 bp, synthetic construct; identical to SEQ ID NO: 8
  misc_feature 1..7: note = DNA
  misc_feature 8..15: note = RNA
  misc_feature 16..34: note = DNA
  source 1..34: mol_type = other DNA

SEQ ID NO: 35 - DNA, 35 bp, synthetic construct; identical to SEQ ID NO: 9
  source 1..35: mol_type = other DNA
  misc_feature 22..35: note = Capture region

SEQ ID NO: 36 - DNA, 21 bp, synthetic construct
  source 1..21: mol_type = other DNA
tacatcgtgtgatgctatgcc

SEQ ID NO: 37 - DNA, 48 bp, synthetic construct
  source 1..48: mol_type = other DNA
  misc_feature 6..11: note = XhoI restriction site
  misc_feature 35..48: note = Capture region
atatactcgagttagatcgcatggcatagcatgatcgcgtacaatacg

SEQ ID NO: 38 - DNA, 101 bp, synthetic construct
  source 1..101: mol_type = other DNA
tccacgtccaggtccnnnntttcctggacgtggacgtattgtacgcgatctatgaacgtacatcgtgtgatgctatgccatgcgatctcaataatatatat

SEQ ID NO: 39 - DNA, 69 bp, synthetic construct
  misc_feature 1..7: note = DNA
  misc_feature 8..15: note = RNA
  misc_feature 16..69: note = DNA
  source 1..69: mol_type = other DNA
atatttaatattattagatcgcatggcatagcatcacacgatgtacgttcatagatcgcgtacaatacg

SEQ ID NO: 40 - DNA, 60 bp, synthetic construct
  source 1..60: mol_type = other DNA
tcgacgtccacctttttccacgatttttggtggacgtcgacgtattgtacgcgatctatg

SEQ ID NO: 41 - DNA, 57 bp, synthetic construct
  source 1..57: mol_type = other DNA
tcgacgtccacctttttccacgatttttggtggacgtcgacgtattgtacgcgatct

SEQ ID NO: 42 - DNA, 56 bp, synthetic construct
  source 1..56: mol_type = other DNA
tcgacgtccacctttttccacgatttttggtggacgtcgacgtattgtacgcgatc

SEQ ID NO: 43 - DNA, 54 bp, synthetic construct
  source 1..54: mol_type = other DNA
tcgacgtccacctttttccacgatttttggtggacgtcgacgtattgtacgcga

SEQ ID NO: 44 - DNA, 53 bp, synthetic construct
  source 1..53: mol_type = other DNA
tcgacgtccacctttttccacgatttttggtggacgtcgacgtattgtacgcg

SEQ ID NO: 45 - DNA, 52 bp, synthetic construct
  source 1..52: mol_type = other DNA
tcgacgtccacctttttccacgatttttggtggacgtcgacgtattgtacgc

(19) United States
(12) Patent Application Publication
(10) Pub. No.: US 2026/0234604 A1
(43) Pub. Date: Aug. 13, 2026

(54) COMPOSITIONS AND METHODS FOR MOLECULAR BARCODING

(71) Applicant: Oregon Health & Science University, Portland, OR (US)

(72) Inventor: Corey M. Dambacher, Portland, OR (US)

(73) Assignee: Oregon Health & Science University, Portland, OR (US)

(21) Appl. No.: 19/160,405

(22) PCT Filed: Feb. 28, 2024
(86) PCT No.: PCT/US2024/017772

§ 371 Date: Aug. 27, 2025

Related U.S. Application Data
(60) Provisional application No. 63/487,575, filed on Feb. 28, 2023.

(51) Int. Cl. C12N 15/10

(52) U.S. Cl. CPC C12N 15/1065

(57) ABSTRACT

Describe herein are nucleic acid oligonucleotides (oligos) including Hairpin Tag nucleic acid molecules and DNA/RNA Hybrid Hairpin Tag nucleic acid molecules, and capture oligo (CO) nucleic acid molecules and DNA/RNA chimeric CO nucleic acid molecules; spatially encoding capture arrays; use of the oligos in coding and decoding features; and use of the oligos and/or arrays and other provided comments, including in methods and workflows for spatial encoding and quantification of targets, such as biomarkers in biological samples.

### CROSS-REFERENCE TO RELATED APPLICATION

This is the 371 National Phase of International Application No. PCT/US2024/017772, filed Feb. 28, 2024, which claims priority to and the benefit of the earlier filing of U.S. Provisional Application No. 63/487,575, filed on Feb. 28, 2023, which is incorporated by reference herein in its entirety.

### INCORPORATION BY REFERENCE OF SEQUENCE LISTING

A computer readable file, entitled “0046-0080US_ST26.xml” created on or about Aug. 27, 2025, with a file size of 65,536 bytes, contains the Sequence Listing for this application and is hereby incorporated by reference in its entirety.

### FIELD OF THE DISCLOSURE

The present disclosure relates generally to methods and systems for analyzing targets, such as biological samples and molecules. More particularly, it relates to molecular barcodes and spatially encoded analyses.

### BACKGROUND OF THE DISCLOSURE

Determining the identity and/or location(s) of targets molecules (such as proteins or nucleic acids) in the sample can be vital for clinical applications, diagnostics, and biomedical research. In situ hybridization (ISH), immunohistochemistry, laser capture microdissection, and such technologies, enable visualization of the locations of target molecules within a sample, such as a biological sample.

The identities of target molecules also may be determined using methods (such as stochastic barcoding) that label target molecules, and track them through process of amplification and/or sequencing. However, there remains an on-going need for methods and systems that reliably correlate the identity of target molecule(s) with their location(s) within a sample, such as a substantially two-dimensional (2D) biological sample.

Spatial biology platforms are revolutionizing the study of biology and are rapidly becoming indispensable for the understanding and treatment of cancer. However, the platforms on the market now, whether for proteomics profiling or transcriptomics analyses, are costly and are not scalable for widespread adoption. Current imaging-based approaches used for proteomics profiling may provide exquisite detail but are slow and are limited in the number of protein targets that can be analyzed on a single tissue slide. Imageless systems allow simplified data acquisition via next generation sequencing (NGS) readouts, but these platforms have difficulty achieving subcellular resolution, have a high cost/assay (due to the need to purchase dedicated instruments), and require multiple manual intervention steps.

### SUMMARY OF THE DISCLOSURE

Described herein is a scalable, low-cost, image-free spatial platform with sub-cellular resolution that is suitable for use, for instance, in basic research and clinical laboratory environments. The described barcoded tags and isothermal workflow enable information transfer to a spatially encoded array in a single, hands-free step. The workflow is non-destructive to the source material (e.g., a fresh froze or fixed tissue sample) and can function without specialized instrumentation to produce digital “images” of component (e.g., protein) presence and abundance at sub-cellular resolution. The described platform enables coordinate spatial and architectural analysis, for instance of cellular proteomic profiles, for deployment into research as well as clinical labs. Also provided are spatial capture arrays for the spatial encoding of information retrieved from bound probes (e.g., antibody probes) conjugated with tags as disclosed herein, as well as isothermal spatial encoding workflows that provide decoding of the retrieved information, for instance by NGS, which enable digital display of protein profiles in biological samples for example.

Provided herein are DNA/RNA hybrid tags, along with workflows for spatial encoding, identification, and/and quantification of biomarkers in biological samples.

A first embodiment is a DNA/RNA hybrid hairpin tag, having a structure as illustrated or described herein. By way of example, the DNA/RNA hybrid hairpin tag may include (in 5′ to 3′ order) an attachment moiety, a string of RNA bases, a Tag PCR handle, a tag ID barcode, a Tag PCR handle complement sequence, and a string of DNA bases complementary to at least a portion of the string of RNA bases. For instance, example DNA/RNA hybrid hairpin tags have a structure as illustrated in FIG. 1A, or as having the sequence of Tag v1, Tag v2, Tag v3, Tag v5, or Tag v6 (SEQ ID NOs: 3-7, respectively).

Also provided are released hairpin tags derived from the DNA/RNA hybrid hairpin tags described herein, including for instance as illustrated in FIG. 1B.

An additional embodiment is a tagged element (structure, component, etc.), which tagged element includes an element to which is attached (through the attachment moiety) a DNA/RNA hybrid hairpin tag illustrated or described herein. In examples of this embodiment, the element includes one or more of: a biological molecule (such as a protein or a nucleic acid), a cell or tissue, an affinity molecule (such as an antibody), a bead, or another addressable feature.

Yet another embodiment is a capture oligo (CO) having a structure as illustrated or described herein. For instances, exemplary COs include (in 5′ to 3′ order) an attachment moiety, a first string of DNA bases, a tag ID barcode, and a second string of DNA bases complementary to at least a portion of the first string of DNA bases. By way of example, a CO may have a structure as illustrated in FIG. 2 or FIG. 3.

Also provided are any of these capture oligos, which is attached to a capture feature through the attachment moiety.

In any embodiment of the CO, or which includes a CO, the CO may further include a conditionally cleavable element. Optionally, this may be positioned such that it allows release of the CO (or a substantial portion of the CO) from an element or surface to which a CO is attached (such as by the attachment moiety).

Another embodiment is a capture element (structure, component, etc.), which capture element includes an element to which is attached (through the attachment moiety) a CO. By way of example, the element includes a bead or another addressable capture feature.

It is contemplated that, in embodiments, the provide capture element is one capture element within an array of different capture elements, and wherein the CO on each of a plurality of the different capture elements of the array each include a different tag ID barcode.

Also provided are capture pairs (e.g., a pair of oligonucleotides), which capture pair include: a DNA/RNA hybrid hairpin tag having a structure as illustrated or described herein, or a released hairpin tag derived from the DNA/RNA hybrid hairpin tag; and a capture oligo (CO) having a structure as illustrated or described herein, wherein the sequence of the DNA/RNA hybrid hairpin tag and the sequence of the CO are at least partially complementary, such that when the hairpin tag is released in proximity to the CO, the released hairpin tag is captured by the CO. Such capture pairs are illustrated herein, for instance in the Figures and corresponding description, as well as in Appendix A included in priority U.S. Provisional Application No. 63/487,575, filed on Feb. 28, 2023.

Another embodiment is a method for detection and/or quantification of targets in a substantially two-dimensional (2D) sample, which method is as described or illustrated herein. The substantially 2D sample may be a tissue thin section, or another slice of biological tissue (such as an archival tissue slice), a substantially 2D array of biological samples (such as may be applied to a slide or other surface, including for instance cell extracts or synthetic mixtures or the like), and so forth. The substantially 2D sample may optionally be homogenous (as to content, such as biological macromolecules) across its surface area, but more often it is heterogenous such that some target(s) are found only in some positions or with varying concentration at some positions. Thus, embodiments of the provided methods allow localization of target(s) within the 2D surface of the sample being analyzed.

Also provided are methods for detection and/or quantification and/or localization of target(s) in a substantially two-dimensional (2D) sample, which is a “one-pot” method (where multiple chemical reactions and/or enzymatic reactions occur, either simultaneously or in sequence) carried out essentially at a single temperature (that is, isothermally).

Examples of these methods for detection and/or quantification and/or localization of targets in a substantially two-dimensional (2D) sample are illustrated (in whole or in part) in FIGS. 4A, 4B, 5A, 5B, 6, 7A, 7B, 8, 9A-9E, 12, 13, or Appendix A included in priority U.S. Provisional Application No. 63/487,575, filed on Feb. 28, 2023.

In any of the provided methods, embodiments include the enzymatic activity of one or more of RNase H, DNA polymerase, reverse transcriptase, RNA polymerase, and/or one or more restriction enzyme(s). Optimally, these enzymatic activities may take place in a single container (a “one-pot” method) and/or at a single temperature (isothermal method).

Any of the provided method embodiments may further include sequence analysis of a plurality of nucleic acid molecules containing one or molecular ID tag(s).

Another embodiment is a spatially-encoded capture array, substantially as described or illustrated herein. For instance, such spatially-encoding capture array may be as illustrated in FIG. 10 or FIG. 11, or in Appendix A of priority U.S. Provisional Application No. 63/487,575, filed on Feb. 28, 2023. The provided spatially-encoding capture arrays of embodiments include capture elements embedded in a biomolecule-permeable matrix. In examples, the capture elements include beads; the biomolecule-permeable matrix is a gel; or both. Optionally The spatially-encoded capture array is provided in the form of a pliable “sticker”, which for instance is intended to be used in direct contact with a substantially 2D sample, for analysis of targets within that sample. Examples of such analysis methods are described.

Yet another embodiment is a spatial encoding workflow essentially as described or illustrated herein. Examples of such spatial encoding workflows are illustrated in FIG. 12. or FIG. 13, or Appendix A of priority U.S. Provisional Application No. 63/487,575, filed on Feb. 28, 2023.

Also described is a spatially-encoding feature array having the “grid within a grid” arrangement illustrated in FIG. 10, and describe herein.

Additional embodiments include a computer readable medium or digital resource, or a digital database, containing spatial location information for features of a spatially-encoding array as describe herein. By way of example, the computer readable medium or digital resource, or a digital database, in some instances contains spatial location information for substantially all the features of the spatially-encoding array.

Also provided is use of the computer readable medium or digital resource, or a digital database, to provide a user with location information for one or more targets correlated with the spatial location information of the spatially-encoding array. For instance, in some cases the correlation arises through use of the spatially-encoding array in a workflow or method as described or illustrated herein.

Another embodiment is a method for isothermal spatial encoding of biological samples, substantially as described or illustrated herein.

Use of a DNA/RNA hybrid hairpin tag as described or illustrated herein, for transcriptomic analysis of a biological sample, is also described.

Also described are spatial encoding surfaces, such as a capture array, for instance a bead-based capture array, substantially as described or illustrated herein.

Provided herein are Hairpin Tag nucleic acid molecules that include, functionally connected in 5′ to 3′ order, parts A-B-C-D-E, wherein: part A includes a Cleavable Site including either: (1) a string of RNA bases, or (2) a string of DNA bases including a restriction enzyme (RE) recognition site; part B includes a string of DNA bases including a Tag PCR Handle; part C includes a string of DNA bases including a Tag ID Barcode, and which string of DNA bases forms a portion of the loop of the hairpin; part D includes a string of DNA bases having the reverse complementary sequence of the Tag PCR Handle, thereby forming a portion of the stem of the hairpin; and part E includes a string of DNA bases having the reverse complementary sequence of at least a portion of either (1) the string of RNA bases of part A or (2) the string of DNA bases including the RE recognition site, thereby forming a portion of the stem of the hairpin.

Also provided are sets of two or more Hairpin Tag nucleic acid molecules as described and/or DNA/RNA Hybrid Hairpin Tag nucleic acid molecules as described, where each of the two or more nucleic acid molecules has a unique Tag ID Barcode sequence. In examples of such sets, the set further includes at least one Attenuation Tag-like nucleic acid molecule, which Attenuation Tag-like nucleic acid molecule differs from a Hairpin Tag nucleic acid molecule or DNA/RNA Hybrid Hairpin Tag nucleic acid molecule of the set by lack of a functional Cleavable Site. By way of example, there are provided sets of two or more Hairpin Tag nucleic acid molecules and/or DNA/RNA Hybrid Hairpin Tag nucleic acid molecules, wherein the Attenuation Tag-like nucleic acid molecule differs from a Hairpin Tag nucleic acid molecule or DNA/RNA Hybrid Hairpin Tag nucleic acid molecules of the set by (1) having DNA bases in place of the RNA bases of part A, or (2) lacks the RE recognition site of part A.

Also provided are Tagged Probes, which including a Probe Molecule to which is attached through the attachment moiety to a Hairpin Tag nucleic acid molecule as described, or to a DNA/RNA Hybrid Hairpin Tag nucleic acid molecule as described. By way of example, the Probe Molecule includes an affinity molecule having a binding affinity for a target molecule. Optionally, the affinity molecule includes an antibody binding domain having affinity for an antigen, and the target molecule includes the antigen.

Another embodiment is a Released Hairpin Tag nucleic acid molecule derived from a Hairpin Tag as described, or from a DNA/RNA Hybrid Hairpin Tag as described, wherein the Released Hairpin Tag has been separated from the attachment moiety by enzymatic action of a Restriction Endonuclease or a RNase H enzyme.

Yet another embodiment is a Capture Oligo (CO) nucleic acid molecule that includes, functionally connected in 5′ to 3′ order, parts 1-II-III-IV, wherein: part I includes a Cleavable Site including either: (1) a single RNA base or a contiguous string of RNA bases, or (2) a string of DNA bases including a restriction enzyme (RE) recognition site; part II includes a string of DNA bases including a CO PCR Handle; part III includes a string of DNA bases including a Spatial Barcode; and part IV includes a string of DNA bases including a Tag Capture Region. Optionally, the CO nucleic acid molecule further includes an attachment moiety conjugated to the 5′ end of part 1. For instance, the attachment moiety in instances provides amine-reactive crosslinker activity or thiol-reactive crosslinker activity.

Yet another embodiment is a set of two or more CO nucleic acid molecules of any of the provided CO nucleic acid molecules, and/or DNA/RNA chimeric CO nucleic acid molecules of any the provided DNA/RNA chimeric CO nucleic acid molecules, where each of the two or more nucleic acid molecules has a unique Spatial Barcode sequence.

Yet another provided embodiment is a Spatially Encoded Capture Feature, which include a Capture Feature to which is attached through the attachment moiety a CO nucleic acid molecule as provided or a DNA/RNA chimeric CO nucleic acid molecule as provided.

Also described are Capture Pairs, which including: a Hairpin Tag nucleic acid molecule as provided, or a DNA/RNA Hybrid Hairpin Tag nucleic acid molecule as provided, or a Released hairpin tag nucleic acid molecule as provided; and a Capture Oligo (CO) nucleic acid molecule of as provided or a DNA/RNA chimeric CO nucleic acid molecule as provided, or an Attached CO as provided, wherein the sequence of the Hairpin Tag nucleic acid molecule and the sequence of the CO nucleic acid molecule are at least partially complementary, such that when the Hairpin Tag nucleic acid molecule is released from its attachment moiety in proximity to the CO nucleic acid molecule, the released Hairpin Tag nucleic acid molecule is captured by sequence complementarity bonding at the 3′ ends to the CO nucleic acid molecule, such that resultant complex of the released Hairpin Tag nucleic acid molecule and the CO nucleic acid molecule is competent for a downstream extension reaction by a polymerase enzyme.

Yet another embodiment is a Spatially Encoding Capture Array, which includes a defined array of spatially-addressed capture features, wherein each capture feature includes: a spatially-identifiable feature including: a pre-defined, addressable location on a substantially two-dimensional solid surface; or a bead or other similar separate, solid capture object; and attached at each feature, multiple copies of a CO nucleic acid molecule as provided or a DNA/RNA chimeric CO nucleic acid molecule as provided, wherein the CO nucleic acid molecules at each feature have a unique Spatial Barcode sequence compared to the CO nucleic acid molecules at other features in the array.

Another embodiment is a semi-ordered Spatially Encoding Capture Array, which includes: a grid of spatially addressable locations, each of which is labeled with oligonucleotides having a unique X-Y coordinated sequence, which oligonucleotides are applied to the spatially addressable locations by splint ligation of: a x-coordinate adapter oligonucleotide, which x-coordinate adapter oligonucleotide is used to label all spatially addressable locations within a row of the grid; and a y-coordinate adapter oligonucleotide, which y-coordinate adapter oligonucleotide is used to label all spatially addressable locations within a column of the grid.

Also provided are methods for detection and/or quantification and/or localization of targets in a substantially two-dimensional (2D) sample, which methods involve: contacting a substantially 2D sample with at least one tagged probe to produce a substantially 2D stained sample, which tagged probe includes: a Hairpin Tag nucleic acid molecule including an attachment moiety and a Tag ID Barcode, or a DNA/RNA Hybrid Hairpin Tag nucleic acid molecule including an attachment moiety and a Tag ID Barcode; and a Probe Molecule attached through the attachment moiety to the Hairpin Tag nucleic acid molecule including a Tag ID Barcode, or the DNA/RNA Hybrid Hairpin Tag nucleic acid molecule including a Tag ID Barcode; contacting a surface of the substantially 2D stained sample with a permeable, spatially encoding capture array to form a sample-array sandwich, which spatially encoding capture array includes: a plurality of spatially identifiable features; and attached at each spatially identifiable feature, multiple copies of a Capture Oligo (CO) nucleic acid molecule or a DNA/RNA chimeric CO nucleic acid molecule, wherein the CO nucleic acid molecules at each feature have a unique Spatial Barcode sequence compared to the CO nucleic acid molecules at other features in the array; placing a flow cell or other solution-containing cover over the sample-array sandwich to form an enclosure containing the sample-array sandwich; adding to the enclosure a solution including reaction components to form a reaction mixture, which components include: a cleavage enzyme selected from a RNAseH or at least one restriction endonuclease (RE); at least one polymerase; a mixture of ribonucleoside tri-phosphates (rNTPs) and/or deoxynucleotide triphosphates (dNTPs); Mg²⁺ ions; and a buffering agent; incubating the sample-array sandwich in contact with the reaction mixture at an assay temperature for 30-60 minutes, to form a reaction product mixture; removing at least a portion of the reaction product mixture from the enclosure; and analyzing the reaction product mixture to detect and/or quantify and/or define the location of targets in the substantially 2D sample. Optionally, in such methods the enclosure includes a flow cell. Embodiment of the provided method provide location information for more than one target within the substantially 2D sample.

Yet another embodiment is a spatial encoding workflow, including: contacting a hybrid RNA/DNA tag including a spatial barcode conjugated to an antibody probe with a substantially two-dimensional (2D) tissue sample, to produce a stained sample; placing a capture array including capture features in contact with the stained sample to produce a sample/array sandwich; placing a fluid-containment enclosure on top of the sample/array sandwich; introducing assay solution including active RNAseH and active polymerase into the fluid-containment enclosure, thereby bringing the assay solution into contact with the sample/array sandwich; incubating the sample/array sandwich in the sample solution at a temperature and for a period of time sufficient for the RNAseH activity to at least partially digest the hybrid RNA/DNA tag to produce a cleaved tag and thereby releasing the cleaved tag into the assay solution in proximity to a capture feature; permitting interaction of the cleaved tag with a capture oligo (CO) on the proximal capture feature to provide a captured cleaved tag; incubating the sample/array sandwich in the sample solution at a temperature and for a period of time sufficient for the polymerase activity to extend the captured cleaved tag using the CO as template, to produce an extension product; cleaving the extension product, upon sufficient extension to produce a complementary RNA/DNA region based on the RNA base(s) in the CO, with RNAseH in the sample solution, thereby releasing the full-length extension products; collecting at least a portion of the full-length, released extension products; and amplifying and/or sequencing at least one of the full-length, released extension products.

Also provided are computer readable medium or digital resources, and digital databases, containing spatial location information for features of a spatially encoding array as described herein. Use of the provided computer readable medium or digital resource, or a digital database, to provide a user with location information for one or more targets correlated with the spatial location information of the spatially encoding array, is also disclosed. By way of example uses of the provided computer readable medium or digital resource, or a digital database, the correlation arises through use of the spatially encoding array in a workflow or method as described or illustrated herein.

Yet another embodiment is a kit useful for carrying out one of the methods provided herein. Representative kits include one or more of: two or more Hairpin Tag nucleic acid molecules as described; two or more DNA/RNA Hybrid Hairpin Tag nucleic acid molecules as described; a described set of two or more Hairpin Tags; a set of two or more Tagged Probes of as described; two or more Capture Oligo (CO) nucleic acid molecules as described; two or more DNA/RNA chimeric CO nucleic acid molecules as described; a described set of two or more CO nucleic acid molecules; a described set of two or more DNA/RNA chimeric CO nucleic acid molecules; two or more CO nucleic acid molecules, each attached to a capture feature, as described; at least one spatially encoding capture array as described; at least one semi-ordered spatially encoding capture array as described; or a spatial encoding surface as described.

Also provide are kit embodiment, further including one or more of: a container in which is contained functional RNAseH enzyme; a container in which is contained functional polymerase enzyme; a container in which is contained functional restriction enzyme; a container in which is contained one or a mixture of ribonucleoside tri-phosphates (rNTPs) and/or deoxynucleotide triphosphates (dNTPs); a container in which is contained a solution including Mg²⁺ ions; or a container in which is contained a buffer solution.

### BRIEF DESCRIPTION OF THE DRAWINGS

FIG. 1A: Hybrid hairpin tag structure shown with exemplary sequence. An exemplary nucleic acid tag (Tag1 v6; SEQ ID NO: 7) that contains both RNA and DNA bases within a hairpin (stem-and-loop) structure is shown. The 5′-end of the tag contains a flexible linker that is attached to a molecule (X in the figure), which may be bound to a target immobilized on a surface. Going from 5′ to 3′, the linker is followed by a stretch of RNA bases, a downstream constant (across a set of hybrid hairpin tags) PCR handle, 1-3 G bases used for spacing the primer landing site from the unique tag barcode, a unique tag barcode placed within the loop of the hairpin (unique within a set of hybrid hairpin tags), which is followed by a 3′ region that is complementary to (that is, the reverse complement of) the 5′ region of the hairpin tag. In the formed hairpin tag, the RNA and DNA bases of the tag generate a DNA/RNA hybrid region (a portion of the stem of the hairpin) that is cleavable by RNase H.

FIG. 1B: Once cleaved by RNAse H, the tag is released from the molecule (X). Digestion of the RNA region of the tag (to produce, e.g., SEQ ID NO: 8) by RNAse H exposes a region of ssDNA bases within the hairpin stem that is designed for capture onto a capture oligo (CO).

FIG. 2: Capture of cleaved hairpin tag by capture oligo (CO) on capture beads. 3′-ends of capture oligos (oligo nucleotides designed for capture of released hairpin tags) contain a capture region that is complementary to the exposed DNA bases on released tags, following RNase H digestion (see FIGS. 1A-1B). The sequence of an exemplary capture region is shown (italicized in figure), bound to the captured hairpin tag (SEQ ID NO: 6). In embodiments, capture oligos are attached at their 5′-ends to a capture feature (illustrated here with a bead).

FIG. 3: Capture bead with capture oligo (CO) structure. The structure of an exemplary single-stranded CO containing a flexible linker at the 5′-end for attachment to a capture feature (illustrated here with a bead), followed by a 5′ PCR handle, a spatial barcode, and a 3′ capture region is shown. COs may also contain a unique molecular identifier (UMI), for instance as a stretch of randomized bases or scattered throughout the spatial barcode region. Attachment of the capture oligo to the capture feature (bead shown here) is conditionally cleavable, for instance by the inclusion of a photo- or chemically-cleavable linker, or by the inclusion of RNA bases or half of a dsDNA restriction enzyme site, as described herein. The 2D location of each capture feature is pre-determined (e.g., within an array) and this information is recorded within the spatial barcode region of the COs. All COs within/located at a capture feature share a unique spatial barcode corresponding to the location of the capture feature (e.g., bead, addressable region of a solid surface, etc.) to which they are attached.

FIG. 4A: Hairpin tag release in proximity to capture feature(s). A pool of molecules (such as a probe panel or library), whereby each unique molecule in the pool is encoded with a specific molecule-identifying DNA barcode sequence (within the tag loop region), is incubated with a sample immobilized on a surface. The sample is then washed to remove any un-bound and non-specifically bound molecules. One or more enzymes that collectively have RNase H and polymerase activity are then introduced in a common buffer with appropriate cofactors (such as dNTPs, Mg²⁺). Tags (molecular IDs, “released tags”, “hairpin tags”) are thereby released from molecules (illustrated as X in the figure) that are bound to targets on a surface. This release is carried out in proximity to COs that are attached to capture features (illustrated as a bead).

FIG. 4B: Hairpin tag capture and extension. In an isothermal “one-pot” reaction, hairpin tags are released from the sample surface, captured onto nearby COs (based on sequence of the capture region of the CO, as well as by proximity of the CO to the point from which the tag was released), and 3′-ends of captured tags are extended by the introduced polymerase. In some embodiments, 3′-ends of COs are blocked. Inclusion of a DNA polymerase within the one-pot reaction prevents “hopping” of tags to other nearby capture features, as the melting temperature of the interaction (between the captured hairpin tag and the capture sequence of the capture oligo) is increased significantly by the extension reaction. Such hopping, if it were to occur, might contribute to loss of location fidelity in the analysis (e.g., though increased diffusion).

FIG. 5A: Alternative construction of tags for compatibility. Alternative tags can be constructed for compatibility with the described isothermal one-pot reactions. Illustrated in this figure, by simply changing the base composition of the tag to include RNA bases between the linker region of the tag and the molecule ID region, a compatible and cleavable tag can be generated. In this way, other tag types can be used in the same isothermal one-pot reaction workflow as described in FIG. 4B.

FIG. 5B: Alternative tag capture and extension. Alternatively designed tags outlined in FIG. 5A can be captured onto proximal COs on capture features (such as beads), and extended by the polymerase included in the isothermal one-pot reaction. As with hairpin oligo capture (illustrated in FIGS. 4A-4B), 3′-ends of COs may be blocked or extended during the one-pot reaction when capturing alternatively designed tags.

FIG. 6: Alternative tag design for compatibility and isothermal amplification. Another type of tag design is also contemplated for use in a similar isothermal one-pot reaction. As illustrated, a promotor region that is recognized by an RNA polymerase (T7 polymerase, for example) can be designed for incorporation into tag sequences at one end or at both ends of the tag. A dsDNA tag bearing two molecules (X and Y), whereby each molecule is encoded (labeled, identified) within a different strand of the dsDNA tag, as shown. Here, opposing T7 promotor regions are installed to create amplifiable templates from both strands of the tag. These probes can be bound to a sample that is immobilized on a surface, then the surface is washed to remove unbound probes, and the washed surface exposed to a solution containing T7 polymerase with appropriate cofactors for RNA transcription. This reaction can be performed in proximity to capture features containing Cos (e.g., as provided herein), within an isothermal one-pot reaction containing a reverse transcriptase (RT) enzyme with appropriate cofactors for DNA extension.

FIG. 7A: Conversion of ssDNA tags for compatibility and isothermal amplification. Tags of existing DNA-tagged molecules can be converted for compatibility with the herein described methods of isothermal information transfer, through installation of a promotor region that is recognized by an RNA polymerase (T7 polymerase for example). A commonly used ssDNA tag structure is shown containing two constant regions (often used as PCR handles) flanking a sequence (Molecule ID) encoding (labeling, identifying) the molecule (X) that is attached through a linker at the terminal end of the tag (5′ shown here). This adapter can be installed before or after the tagged probes have been incubated with the target sample (containing target molecules). Likewise, an extension reaction to generate dsDNA tags through extension of adapter 3′ ends may be performed before or after probes have been incubated with the sample. Following probe binding, unbound probes are removed by washing, and the probe-bound surface would be exposed to a solution containing T7 RNA polymerase and a RT enzyme with appropriate cofactors. This transcription reaction can be performed in proximity to capture features containing COs, within an isothermal one-pot reaction containing a reverse transcriptase (RT) enzyme with appropriate cofactors for DNA extension.

FIG. 7B: Conversion of dsDNA tags for compatibility and isothermal amplification. Tags of existing DNA-tagged molecules can be converted for compatibility with isothermal information transfer through the installation of a promotor region that is recognized by an RNA polymerase (T7 polymerase for example). A commonly used dsDNA tag structure is shown containing two constant regions (often used as PCR handles) flanking a sequence (Molecule ID) encoding (labeling, identifying) the molecule (X) that is attached through a linker at the terminal end of the tag (5′ shown here). This adapter can be installed before or after the tagged probes have been incubated with the sample. Following probe binding, unbound probes would be removed by washing, and the probe-bound surface would be exposed to a solution containing T7 RNA polymerase and a RT enzyme with appropriate cofactors. This transcription reaction can be performed in proximity to capture features containing COs, within an isothermal one-pot reaction containing a RT enzyme with appropriate cofactors for DNA extension.

FIG. 8: Capture of RNA produced from tag templates. As the transcribed RNAs are released into solution, they are captured onto 3′-ends of capture oligos attached to proximal capture features (illustrated with beads), and 3′-ends are immediately extended by the RT enzyme present in the same reaction mixture. Once the RNA strands are copied onto 3′-ends of capture oligos via the extension reaction, they are destroyed by RNase H activity (contained within the RT enzyme, or by the inclusion in the reaction mixture of an enzyme containing RNase H activity, such as E. coli RNase H). The resulting extension products contain spatial information provided by the spatial barcodes, the tag information, as well as 3′- and 5′-PCR handles for downstream amplification. Spatially encoded products can be released from capture features in a variety of ways, such as outlined in FIGS. 9A-9E.

FIG. 9A: Release of double-stranded products from capture features by RNase H. Following extension of tag 3′-ends on capture oligos to generate spatially-encoded dsDNA products, these spatially-encoded products can be released into the surrounding solution within the same isothermal one-pot information transfer reaction workflow. Here, RNA bases are included within the 5′-end of capture oligos, such that following extension by the DNA polymerase (specifically, an RNA- and DNA-dependent DNA polymerase; Maxima™ for example, available from Thermo Scientific), a DNA/RNA hybrid region is generated which becomes a substrate for RNase H activity in the reaction. These products are cleaved from capture features by RNase H and released into the reaction solution.

FIG. 9B: Release of double-stranded products from capture features by endonuclease. An alternative way to release spatially-encoded dsDNA extension products from capture features is to include half of a dsDNA restriction enzyme (RE) site within the 5′-end of capture oligos. Following extension of tag 3′-ends on capture oligos, a fully formed dsDNA RE site will be generated. A cognate RE can be introduced during the isothermal information transfer reaction or after the reaction has completed. Upon introduction this RE will cleave spatially-encoded dsDNA products from capture features and these will be released into the reaction solution.

FIG. 9C: Release of double-stranded products from capture features; photo- or chemical. An alternative way to release spatially-encoded dsDNA extension products from capture features is to include a photo- or chemically-cleavable moiety within the linker that attaches the capture oligo to the capture feature. Here, the cleaving agent is introduced after the one pot isothermal information transfer reaction has completed. This method can be combined with other methods for dsDNA capture oligo release (for example via RNase H or RE activity) in known percentages for a variety of specialized applications.

FIG. 9D: Release of single-stranded products from capture features; photo- or chemical. Following capture and extension of transcription products, a single-stranded spatially-encoded DNA product is generated. These products can be released from capture features by the inclusion of a photo- or chemically-cleavable moiety within the linker that attaches the capture oligo to the capture feature. Here, the cleaving agent is introduced after the one pot isothermal information transfer reaction has completed. This method can be combined with other methods for ssDNA capture oligo release (for example via RNase H or RE activity following conversion to dsDNA as outlined below) in known percentages for a variety of specialized applications.

FIG. 9E: Conversion of single-stranded products for release from capture features. Following capture and extension of transcription products, a single-stranded spatially-encoded DNA product is generated. A constant primer can be annealed to the 3′ ends of these oligos, and extended by a DNA polymerase to convert these into spatially-encoded dsDNA products on the capture features. The inclusion of RNA bases or half of a dsDNA RE site at the 5′-end of the capture oligos will enable enzymatic release of these products from capture features following their conversion into dsDNA. Following extension of tag 3′-ends on capture oligos, a fully formed dsDNA RE site will be generated, or a DNA/RNA hybrid region will be generated. Upon exposure to the appropriate enzyme, the products will be released into the reaction solution.

FIG. 10: Exemplary spatially-encoded bead arrays. One major problem with using a capture array composed of small (1-10 μm), tightly-packed capture beads (or monodisperse beads at high density) is the number of different beads that must be used for generating capture arrays of large surface areas. For example, to construct a 1.75 cm×1.75 cm capture array composed of packed 1 μm beads, about 10B (1×10¹⁰) individual 1 μm beads are required to completely cover the surface area. Furthermore, there can be very little redundancy (none ideally) in bead spatial barcodes across the capture array, which in this example would require a capture bead library size of at least 10¹⁶ different beads to reduce the probability of spatial barcode redundancy to about 1 in 10,000. Additionally, libraries of beads designed for visual encoding and decoding methods described here (for example orthogonal cleavage sequencing (OCS) as described in WO 2022/187719, and other libraries that are constructed using a splitting and pooling approach) are not ideally designed to reach such numbers of diversity. The co-construction of NGS barcodes along with visual barcodes as described here, requires that constant bases are used for splint ligation (e.g., as described in Kershaw & O'Keiffe, Methods Mol Biol. 941:257-269, 2012) of each section, which increases the length of the NGS barcode with each round of splitting and pooling (as diversity within the library is increased). Representative applications of this technology will utilize large spatially-encoded arrays (>1 cm×1 cm, and up to 10 cm×10 cm, for instance), and therefore this problem needed to be addressed. To reduce the number of capture beads required to generate large capture array surfaces, the random bead array may be patterned within a grid. The grid configuration enables sub-sectioning of the random bead array into smaller features (35-100 μm features illustrated). When using grid features of 35 μm, and beads of 1 μm diameter, each grid feature will contain about 1,250 individual beads. Likewise, grid features of 100 μm containing packed 3 μm beads, will have about 1,000 individual beads in each feature. Following bead attachment to the surface (loading of the grid), each feature of the grid receives a unique spatial address. To spatially address each feature of the grid, x- and y-coordinate adapters are added to the grid for instance by droplet printing. In brief, x-coordinate adapters (one unique adapter sequence for every row of the grid) are deposited horizontally across the bead array by droplet printing into features of the grid, and these adapters are included within a solution containing a ligase enzyme, as well as a constant nucleic acid splint, for splint ligation to the constant region of NGS barcodes attached to the beads. Likewise, y-coordinate adapters (one unique adapter sequence for every column of the grid) are deposited vertically across the bead array by droplet printing into features of the grid, and these adapters are included within a solution containing a ligase enzyme, as well as a constant nucleic acid splint, for splint ligation to the constant region of x-coordinate adapters already ligated to the NGS barcodes attached to the beads. The y-coordinate adapter also contains a 3′ capture region for capture of released probe tags from the sample. This enables unique addressing of each feature of the grid, and this process can be expanded to generate very large capture arrays containing packed or monodisperse 1 μm beads at high density. To avoid significant redundancy of spatial barcodes across the array in this configuration, bead library diversities of around 1M can be used, even when generating capture array surface areas of 10 cm×10 cm or greater.

FIG. 11A: Bead capture array; capture beads embedded in macromolecule-permeable material (e.g., gel). After reading bead locations within the capture array grid (for instance, using OCS or another method), the location of each bead in the grid is recorded for instance in a software package. NGS capture oligos on beads contain information that is operationally-coupled to the visual barcode sequence, as well as (in embodiments) the x- and y-coordinate adapters used for locational addressing by droplet printing (see FIG. 10). Beads then can be embedded within a gel or other substance that is permeable to macromolecules, but rigid enough to trap the beads in the matrix following (gel) casting or hardening. After embedding the beads, the matrix containing the beads can be removed from the semi-patterned bead array, and this product can be used as a spatially-identified capture array. By way of example, the “sticker” (cast gel/matrix) thickness in various embodiments is between 100 μm and 250 μm, or (for instance, when cast) as thin as 50 μm. In described embodiments, the gels are 0.5%-2% agarose gels cast using 10 mM Tris pH 7.5 buffer, in which high melt temperature agarose powder (e.g., from Thermo Scientific, such as 16500100) is melted. Other contemplated permeable matrices include polyacrylamide (for instance, 4% polyacrylamide, which has been shown to allow adequate diffusion of macromolecules) or different compositions of hydrogels. Optionally, the permeable matrices (gels) may include functionalization(s) for attachment of immobilization features (such as beads) within the matrix (gel).

FIG. 11B: Capture oligo information copied and retained within gel. To produce many capture array gels from a single bead array, extension products generated as copies of NGS capture oligos can be retained within a cast gel. In brief, NGS capture oligos on beads are built in reverse complement orientation, such that the sequence encoding the 3′ capture region is oriented closest to the bead, and the 5′ PCR handle is oriented furthest away from the bead. These reverse complement NGS barcodes can be primed using a common primer that will anneal to the 5′ constant region on all capture oligos, and this primer can be extended by a DNA polymerase to copy the NGS capture oligo information (spatial barcodes). The common primer used for priming and extension of DNA will contain a moiety for polymerization within a macromolecule-permeable substance such as a gel (for example, using a 5′ acrydite-modified primer for polymerization within a polyacrylamide gel). Following the extension reaction, an appropriate unpolymerized (or unhardened) gel can be cast over the beads to co-polymerize or otherwise incorporate 5′ chemical moieties on extension products into the gel. The gel will now contain correctly oriented (copies) of the NGS capture oligos that were built in reverse complement, with their relative locations preserved. The gel can then be removed without the beads, and the semi-patterned bead array can be used for casting more gels by repeating the annealing, extension, and gel casting procedure.

FIG. 12: An exemplary spatial encoding workflow. The isothermal information transfer reaction can be performed in close proximity to (for instance, placed directly in contact with) capture beads embedded within a macromolecule-permeable substance, such as a gel. The use of a macromolecule permeable substance, such as a gel, for the capture array enables introduction of spatial encoding components (enzymes and cofactors), and the release of spatially-encoded products directly through the substance (gel) by diffusion. As illustrated, an anti-Her2 antibody probe was conjugated with Tag v6 (SEQ ID NO: 7; illustrated in FIG. 1A), and used for probing Her2+ cells (SKBR3 cells) immobilized on a glass slide. Following probe incubation, the slide was washed, and a thin layer of solution (~20 μl) containing a DNA polymerase (Maxima RT) with dNTPs in 1×RT buffer was placed on the sample prior to “sticking” (placing, laying) the capture bead array gel (“sticker”) onto the sample (that is, placing it in very close proximity to, for instance in direct contact with, the sample). In this example, capture oligos contained RNA bases within their 5′ regions. A solution containing dNTPs (to avoid dilution of these required cofactors of small molecular weight) and 1×RT buffer (~40 μl) was then placed on the back side of the gel (that is, the side away from the biological sample). Following a 20-minute incubation at 40° C., this solution was removed and kept as a negative control. After removing the negative control solution, a solution containing E. coli RNase H and dNTPs (to avoid dilution of these required cofactors of small molecular weight) in 1× buffer was placed on the back side of the gel matrix. Following a 20-minute incubation at 40° C., this solution was removed and used with the negative control sample in qPCR experiments for quantifying levels of extension product present in each solution. The graph shows that ~130-fold more extension product was retrieved in the RNase H+ sample over the negative control (RNase H−) sample. This suggests that the enzyme required for initiating the isothermal information transfer reaction, RNase H in this case, was able to diffuse across/through the gel to the sample surface, where this enzyme released information contained within probe tags for capture onto capture beads, whereby the DNA polymerase enzyme was able to extend captured tag 3′-ends for extension through the RNA bases of the capture oligo, enabling release of the extended products through the gel. The released products were then harvested by removing the solution from the back side of the gel, allowing their analysis.

FIG. 13: Illustration of a simple spatial encoding device and workflow. The capabilities described herein, which include the ability to perform the isothermal information transfer reaction and the ability to release extended products from capture beads into solution, enables the simple, straightforward spatial encoding workflow outlined in FIG. 13. Following the addition of probes to a sample immobilized on a surface (tissue section on slide shown here), the sample surface is washed to remove unbound probes, and a macromolecule-permeable spatial encoding capture array (gel sticker) is placed onto the sample. Optionally, a flow cell-like cover can be placed over the encoding surface. This type of device may apply gentle pressure to the encoding surface, to ensure and/or increase the proximity of capture features (within the macromolecule-permeable spatial encoding capture array) with the sample. Once assembled, this device allows for solutions to be flowed over the capture array that has been placed on the tissue/sample, whereby one or more of the spatial encoding reaction components (enzymes and/or cofactors required for one-pot isothermal information transfer and release of spatially-encoded products into solution, as exemplified herein) can be introduced across the macromolecule-permeable gel, and spatially-encoded products can be released through the gel and into the solution contained within the flow cell. Following incubation at the isothermal reaction temperature (e.g., 37-42° C.), spatially-encoded products within the assay solution are retrieved (for instance, by aspiration), and the spatially-encoded products contained within the retrieved solution are analyzed. In embodiments, this involves amplification (e.g., by PCR) for next generation sequencing library preparation.

FIG. 14: “One-Pot” Isothermal Reaction. This figure expands on FIG. 12, and illustrates aspects of embodiments of the assay. In accord with the illustration in FIG. 13, a tissue sample (such as a fresh frozen or formalin-fixed paraffin-embedded (FFPE) tissue section on a glass slide or other surface) is stained with conjugated antibody probes (that is, antibodies to which the DNA/RNA hybrid hairpin tag is conjugated), then a capture array (containing spatially defined capture features to which are attached capture oligos—each of which include a 5′-PCR handle, spatial barcode, UMI, and probe capture region; four capture features are illustrated) is placed on top of the stained tissue. A flow cell is placed on top of the tissue/array sandwich, and assay solution (containing RNAse H and polymerase) is introduced. Action of the RNase H enzyme releases hairpin tag barcodes from bound probes. Released tags diffuse out of the tissue and (A) interact with the spatially encoded array features (that is, released hairpin tag barcodes are captured on the most proximate spatially-defined feature(s) bearing barcoded capture oligos), where (B) it is extended by polymerase in the assay solution, copying the spatial encoding information (that is, the polymerase extends 3′-ends of each probe and thereby copies the UMI, spatial barcode, and 5′-PCR handle of the capture oligo). Upon complete extension, a complementary RNA/DNA region is created, which is then (C) cleaved by RNAse H in the assay solution (Isothermal release probes with template oligos by RNAse H from spatially defined capture features enables PCR amplicon-based Illumina library prep). The released, fully extended tags can then diffuse through the permeable gel matrix and be collected for further analysis, such as amplification and NGS (see the graph in FIG. 12, which illustrates that cleavage release does not happen without RNAse H.

FIG. 15A: An anti-Her2 antibody harboring the HPv6 tag was applied to MCF7 cells (which are Her2 negative). This was done to test background staining issues of the hairpin tagged antibodies. The Her2-HPv6 antibody was visualized with an anti-mouse Alexa 555 secondary to assess background labelling. Three separate block/washing conditions (DMSO, Denhardt's, Salmon Sperm DNA) were tested for their ability to eliminate background staining. Quantification of the images was performed in Volocity® imaging software and data were imported into GraphPad Prism to generate the graphs. (**=p<0.001).

FIG. 15B: Experimental conditions, quantification and graphing were performed as described for FIG. 15A. In this experiment, an anti-ER-HPv6 antibody was tested against SKBR3 cells (ER negative) to assess background staining and blocking methods.

FIGS. 16A-16B. Representation of all 256 loop barcodes. FIG. 16A: The graph was generated from 1 million (randomly selected) reads from the 26,000,000 paired-end (PE) reads that were aligned to the reference genome library; the percentage of representation of each barcode was then plotted. This was done four times, each time with 1 million reads chosen at random, so as to enable the generation of error calculations. The percentage of reads obtained for each of the 256 barcodes was then plotted using GraphPad Prism. Corresponding raw data for reads is provided in Exhibit B of priority Application No. 63/487,575, filed Feb. 28, 2023. FIG. 16B: The illustrated LOGO was generated by uploading 10,000 reads (that had been successfully aligned to the reference genome), to WebLogo (available online at weblogo.berkeley.edu/logo.cgi).

FIGS. 17A-17C. Expansion of spatially-encoded capture array surface area using a dual-coordinate oligo system: Further expansion of spatial encoded array surface areas can be achieved through droplet printing of two barcode types in different combinations. In FIG. 17A, a grid pattern of sub-arrays is shown that outlines a physical map of different combinations of oligos (x- and y-coordinate oligos are shown here), which optionally can be droplet-printed directly into sub-array features, whereby each sub-array contains multiple capture features (for example beads, whereby each bead is coated with a unique capture oligo (CO)). An exemplary sub-array feature size is 100 μm×100 μm, containing ~1,000 3 μm beads or ~10,000 1 μm beads packed closely together, but randomly-arranged within the sub-arrays. In every sub-array, each bead contains many copies of a unique visual barcode that is operationally-coupled to the DNA barcode sequence (VBC) contained within an attached and corresponding capture oligo nucleic acid, which is also represented in many copies. The region of the capture oligo that corresponds to the unique operationally-coupled visual barcode is referred to in the figure as VBC; this region can be further spatially addressed by droplet printing of a unique first oligo barcode (x-coordinate barcode oligo (x-BC) shown here), which is ligated to the VBC using a splinting oligo. This step is further illustrated in FIG. 17B, which shows the addition of two different x-BC oligos (x1 and x2 oligos) by droplet printing onto two adjacent sub-arrays containing bead capture features bearing just the VBC region of the capture oligo. This step is ideally carried out in the presence of an RNA splinting oligo (which may contain a single DNA base at both the 3′ and 5′ ends to improve stability) and the SplintR ligase enzyme (available from NEB M0375S) which recognizes RNA/DNA hybrids for ligation of adjacent DNA strands that are templated by an RNA oligonucleotide. After a wash step, a second spatial coordinate oligo is droplet printed onto each sub-array for ligation to the first spatial coordinate oligo. FIG. 17C shows a second oligo type, a y-coordinate barcode (y-BC) oligo, which is added to each sub-array in perpendicular orientation to which the x-coordinate oligos were added, which is also accompanied by ligation for attachment. This generates unique combinations of two different types of coordinated oligos that can be added and ligated to the VBC oligo to create an expandable surface area of spatially-addressed features to generate a large capture array. Here, the y-BC oligo also contains a capture region for capture of nucleic acids and/or tag oligos from a stained sample.

### REFERENCE TO SEQUENCES

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. All oligonucleotides are shown in 5′ to 3′ orientation. Key: 5AmMC6=5′ flexible 6-carbon atom linker bearing a reactive primary amine (IDT: /5AmMC6/); 5AmMC12=5′ flexible 12-carbon atom linker bearing a reactive primary amine (IDT: /5AmMC12/); 5Phos=5′ phosphorylation, added e.g., so the oligo can be a substrate for T4 DNA ligase; 31nvdT=3′ inverted dT modification, producing a 3′-3′ linkage that inhibits both degradation by 3′ exonucleases and extension by DNA polymerases; 5BioTEG=5′ modification including biotin and a triethyleneglycol (TEG) spacer that increases the oligo-biotin distance to 15 atoms; 5Biosg=5′ biotin, added so the oligo can be captured by binding to avidin; “r” indicates that the following base is RNA rather than DNA; + indicates the following is a locked nucleic acid (LNA) base.

In the Sequence Listing:

| SEQ ID | | |
|---|---|---|
| NO: | Name | Sequence |
| 1 | Ab_DRD_constant | /5AmMC6/ATATATATTATTATTArCrArUrArGrArUrC |
| | | rGrCrGrUrArCrArArUrArCrGTCG |
| 2 | Ab_DRD-Tag1 | /5Phos/ACGTCCACCTTTTTCCACGATTTTTGGTG |
| | | GACGTCGACGTATTGTACGCGATCTATG |
| 3 | DigiSpace HP Tag v1 | /5AmMC6/ATATATATTATTATTArCrArUrArGrArUrC |
| | | rGrCrGrUrArCrArArUrArCrGTCGACGTCCACCTTT |
| | | TTCCACGATTTTTGGTGGACGTCGACGTATTGTA |
| | | CGCGATCTATG |
| 4 | DigiSpace HP Tag v2 | /5AmMC6/ATATATATTATTATTGrUrCrGrCrGrUrAr |
| | (78 nucleotide HP tag) | CrArArUrArCrGTCGACGTCCACCTCCACGATTTG |
| | | GTGGACGTCGACGTATTGTACGCGA |
| 5 | Digispace HP Tag v3 | /5AmMC6/ATATATAGrUrCrGrCrGrUrArCrArArUrAr |
| | (60 nucleotide HP tag) | CrGGTCCACCTCCACGATTTGGTGGACCGTATT |
| | | GTACGCGA |
| 6 | DigiSpace HP tag v5 | /5AmMC6/ATATATATrUrCrGrCrGrUrArCrArArUrAr |
| | | CrGTCCACGTCCAGGTCCACGATTTCCTGGACG |
| | | TGGACGTATTGTACGCGATC |
| 7 | DigiSpace HP tag v6 | /5AmMC12/rUrCrGrCrGrUrArCrArArUrArCrGTCCA |
| | | CGTCCAGGTCCACGATTTCCTGGACGTGGACGT |
| | | ATTGTACGCGA |
| 8 | Ab_DRD-Tag1 cleaved v1 | TCGACGTCCACCTTTTTCCACGATTTTTGGTGGA |
| | | CGTCGACGTATTGTACGCGATCTATG |
| 9 | Ab_DRD-Tag1 cleaved v2 | TCGACGTCCACCTTTTTCCACGATTTTTGGTGGA |
| | | CGTCGACGTATTGTACGCGATCT |
| 10 | Ab_DRD-Tag1 cleaved v3 | TCGACGTCCACCTTTTTCCACGATTTTTGGTGGA |
| | | CGTCGACGTATTGTACGCGATC |
| 11 | Ab_DRD-Tag1 cleaved v4 | TCGACGTCCACCTTTTTCCACGATTTTTGGTGGA |
| | | CGTCGACGTATTGTACGCGA |
| 12 | Ab_DRD-Tag1 cleaved v5 | TCGACGTCCACCTTTTTCCACGATTTTTGGTGGA |
| | | CGTCGACGTATTGTACGCG |
| 13 | Ab_DRD-Tag1 cleaved v6 | TCGACGTCCACCTTTTTCCACGATTTTTGGTGGA |
| | | CGTCGACGTATTGTACGC |
| 14 | DRD1_CO_6sUMI DNA | /5AmMC6/ATATATATTATTGAGATCGCATGGCAT |
| | bases | AGCATCACACGATGTACGTTCATAGATCGCGTA |
| | | CAATACG/3InvdT/ |
| 15 | CO_5_RNA | /5AmMC6/ATATTTArArUrArUrUrArUrUAGATCGCA |
| | | TGGCATAGCAT |
| 16 | CO_3_invT | /5Phos/CACACGATGTACGTTCATAGATCGCGTA |
| | | CAATACG/3InvdT/ |
| 17 | CO_Splint | TACATCGTGTGATGCTATGCC |
| 18 | 5′DRD LNA v4 | +T+CCACGTCCAGGTCC |
| 19 | 3′DRD common v4 | GAGATCGCATGGCATAGCATC |
| 20 | DRD Forward overhang P5- | TCGTCGGCAGCGTCAGATGTGTATAAGAGACAG |
| | tag | GAGATCGCATGGCATAGCATC |
| 21 | DRD Reverse overhang P7- | GTCTCGTGGGCTCGGAGATGTGTATAAGAGACA |
| | tag | GTCCACGTCCAGGTCC |
| 22 | P7-G7 index primer | CAAGCAGAAGACGGCATACGAGATGCCTTAACG |
| | | TCTCGTGGGCTCGG |
| 23 | P5-G7 index primer | AATGATACGGCGACCACCGAGATCTACACTAGG |
| | | AGCTTCGTCGGCAGCGTC |
| 24 | KAPA Primer 1 | AATGATACGGCGACCACCGA |
| 25 | KAPA Primer 2 | CAAGCAGAAGACGGCATACGA |
| 26 | FL T7p short Tag1 | TATATATAATACGACTCACTATAGGGTCCACGTC |
| | | CAGGTCCGACGATTTTCGTATTGTACGCGA |
| 27 | FL T7p short | /5Biosg/TCGCGTACAATACGAAAATCGTCGGACC |
| | Tag1_Biotin_rc: | TGGACGTGGACCCTATAGTGAGTCGTATTATATA |
| | | TA |
| 28 | Short Tag1 need A tail: | /5Phos/TCCACGTCCAGGTCCGACGATTTTCGTA |
| | | TTGTACGCGA |
| 29 | Short Tag1 has A tail: | /5Phos/CCACGTCCAGGTCCGACGATTTTCGTAT |
| | | TGTACGCGA |
| 30 | Short Tag1_rc: | /5Biosg/TCGCGTACAATACGAAAATCGTCGGACC |
| | | TGGACGTGGA |
| 31 | T7p ligation adapter: | TATATATAATACGACTCACTATAGGGT |
| 32 | T7p ligation adapter_rc: | /5Phos/CCCTATAGTGAGTCGTATTATATATA |
| 33 | CO DS primer 1: | TCCACGTCCAGGTCC |
| 34 | CO DS primer 2: | AGATCGCATGGCATAGC |
| 35 | RNA CO no invT: | /5Phos/CACACGATGTACGTTCATAGATCGCGTA |
| | | CAATACG |
| 36 | Biotin TEG-DNA CO no InvT | /5BioTEG/ATATACTCGAGTTAGATCGCATGGCAT |
| | | AGCATGATCGCGTACAATACG |
| 37 | library amplification bias | TCCACGTCCAGGTCCNNNNTTTCCTGGACGTGG |
| | test oligo | ACGTATTGTACGCGATCTATGAACGTACATCGT |
| | | GTGATGCTATGCCATGCGATCTCAATAATATATA |
| | | T |
| 38 | DRD1_CO_6sUMI - RNA | /5AmMC6/ATATTTArArUrArUrUrArUrUAGATCGCA |
| | bases | TGGCATAGCATCACACGATGTACGTTCATAGAT |
| | | CGCGTACAATACG/3InvdT/ |

### DETAILED DESCRIPTION

The hybrid tag structure disclosed here contains both DNA and RNA bases, and can be attached to a solid support, or various macromolecule probes to enable their molecular encoding (identifying, labeling). The tag is designed as a hairpin, such that a stretch of DNA bases complementary to the RNA bases in the tag are held in proximity to generate a stable RNA/DNA hybrid region; RNA bases within the tag are flanked on both sides by DNA bases. These aspects protect the RNA bases within the tag from degradation (mostly by ribonucleases that do not possess RNAse H activity). This structure allows for the tag to be released from its attachment site upon introduction of RNAse H. Additionally, the hairpin makes for a self-contained encoding element, that can be used without the need for hybridization of a recognition strand (this is currently required for DNA paint workflows, as well as other cyclic immunofluorescent (IF) and digital encoding workflows that make use of nucleic acid tagged probes).

Exemplary tag design and experimental examples shown herein relate to using the hybrid hairpin tag for the purpose of encoding antibody probes, which for instance can be used in a workflow for the detection and quantification of protein targets in a tissue section or other substantially two-dimensional (2D) sample arrangement. Embodiments of this workflow also make use of a previously described visual molecular barcode type that enables the identification of many different features (beads) immobilized on a surface (e.g., as published in WO 2022/187719), as well as other methods for providing a visual molecular barcode. Following visual identification, the location of each feature (e.g., each bead) can be determined and recorded (mapped, reliably addressed), and the surface containing the addressed (or mapped) features can be used for spatially-resolved assays. In embodiments, the visual barcodes attached to each feature correspond with (are functionally coupled to) sequence information within capture oligos that is retained throughout a next generation sequencing (NGS) workflow: each bead (or other addressable immobilization feature) contains at least one visual barcode and at least one corresponding capture oligo that is unique to that bead.

In some embodiments, more than one unique visual barcode corresponds to more than one unique capture oligo.

In assays described here, a tissue sample is prepared (such as for standard IHC experiments) and exposed to cocktails or panels of antibody-DNA/RNA hybrid tag-conjugated probes, washed to remove unbound probes, and incubated in a compatible solution for downstream steps (transfer buffer). The surface containing the spatially-resolved beads (or other features) is generated, mapped, and coated with a solution containing (in representative embodiments) transfer buffer, RNAse H and a DNA polymerase at low temperature (below room temperature, for instance on ice). The spatially-resolved feature (e.g., bead) array is then placed “bead-side down” so that the features (beads) are placed in direct contact with the (tissue) sample. By way of example, the (tissue) sample is mounted on/supported by a solid surface, such as a standard microscope slide. The sandwich (bead array/tissue slide) is then incubated at elevated temperature (for instance, RT to 42° C.) for isothermal transfer of tags cleaved (by RNAse H, in exemplary embodiments) from probes bound to the tissue sample onto spatially-encoded capture oligos that are immobilized on features (e.g., beads) of the spatially-encoded (bead) array. In this way, hybrid DNA/RNA hairpin tags can be released from tissue-bound probes, and captured directly onto the most proximal bead/feature relative to each released tag.

Because in embodiments DNA polymerase is also present in the isothermal cleavage/capture solution, each tag, once captured onto the most proximal bead of the array, will have its 3′-end extended to effectively copy the spatial information from the bead onto the tag by DNA extension. 3′-ends of capture oligos are blocked so they are not extendable. This exemplified “touch-down” extension reaction stabilizes the capture of tags and is expected to increase the resolution of the assay by reducing lateral diffusion of released tags. This extension step will solve or significantly reduce the issue of tag “hopping” to and from nearby beads (capture features) following release from probes, because upon extension, the melting temperature (Tm) of the interaction (between the tag oligo and the capture oligo) will increase with every base added to the 3′ end of each captured tag during the extension reaction, as the length of the newly synthesized DNA/DNA duplex interaction increases.

Contained within the released tags (going from 5′ to 3′) is (1) a sequence that acts as a 3′ constant PCR handle; (2) a short sequence corresponding to the probe type (antibody identifying barcode in this example); and (3) a constant capturable region that becomes exposed as a single stranded DNA following RNAse H digestion. The capture oligo on each bead of the spatially-encoded feature (bead) array is attached (immobilized) to the immobilization feature (bead) by its 5′-end through standard linker attachment chemistry (biotin-streptavidin here), and going from 5′ to 3′, includes (in embodiments): (1) a short stretch of 3 DNA bases; (2) eight RNA bases (though this can be varied, as described herein); (3) a constant 5′ PCR handle; (4) a spatial barcode corresponding to the visual barcode on the bead; (5) optionally, a stretch of randomized bases that generate a unique molecular identifier (UMI); and (6) a 3′ constant capture region for capture of released tags.

In examples, the extension reaction copies a unique molecular identifier (UMI), the bead-specific location identifying barcode region, and the 5′ constant PCR handle onto the probe to generate a contiguous sequence containing both the 3′ and 5′ constant PCR handles. “Constant” in all cases herein means that the region/element is common to all probes and capture oligos in the assay.

Since the capture oligos also contain RNA bases at their 5′ ends, after the polymerase extends through the DNA-templated bases of the capture oligo, it will extend through the RNA bases of the capture oligo and thereby generate a DNA/RNA hybrid species. Because RNAse H is also (already) present in the isothermal transfer reaction solution, this newly synthesized DNA/RNA hybrid is cleaved by RNAse H, thereby releasing the capture oligo from the immobilization feature (bead). The released capture oligo, after release, is still hybridized to the full-length extended tag.

Embodiments of this overall process allow for 1) isothermal cleavage of tags from probes bound to the tissue/sample(s) being analyzed; 2) capture of the released tags onto a spatially-encoded array, such as a spatially-encoded bead array; 3) extension of the spatial coordinates onto the tags; and 4) release of the spatially-encoded full-length tags containing both the 5′ and 3′ PCR handles (constant regions) for retrieval and PCR-based amplification of only full-length tags using a set of common primers. The amplification primers can contain, for instance, Illumina flow cell adapter sequences, making the spatially-encoded biomarker assay resolvable and quantitative by next generation sequencing. Step 3) more generally can be viewed as combining tag information with spatial barcode information via an extension reaction, which encompasses additional illustrated embodiments beyond that specified above. An option can be included to block the 3′-end of the tag, which would not be extended during the extension step, as only the CO would be. In such embodiments, the extension product release step is a separate step, because methods rely on there being a second RNA/DNA hybrid generated during the extension step for product release via the one pot reaction.

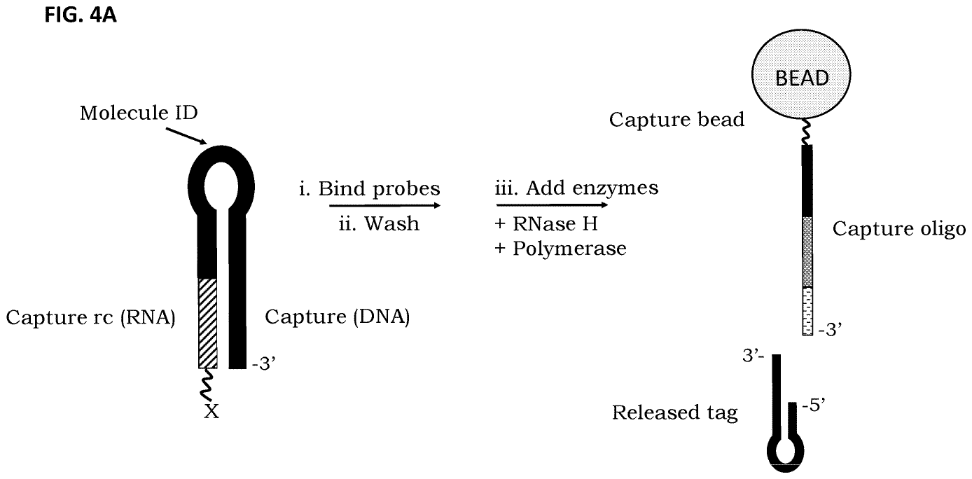

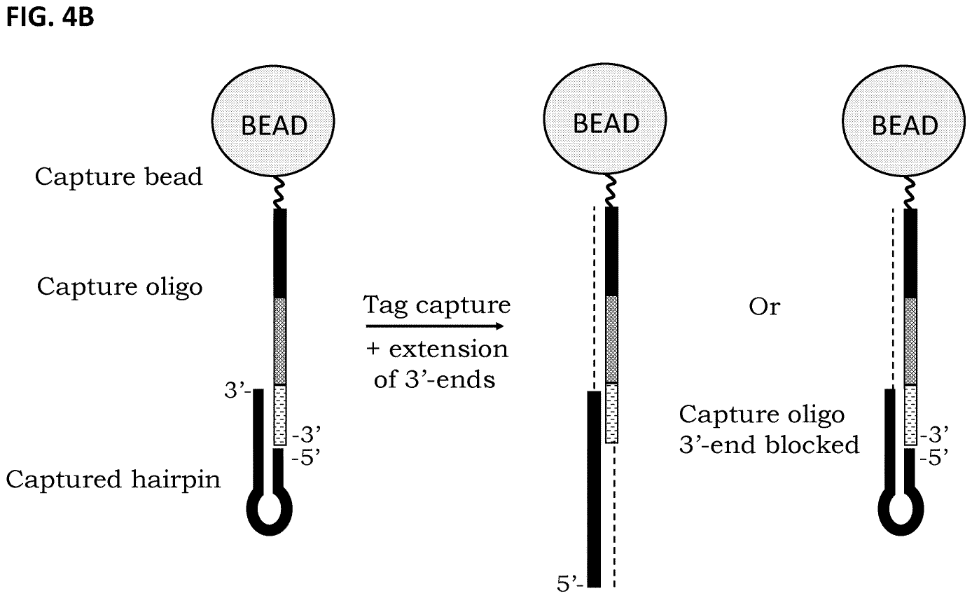

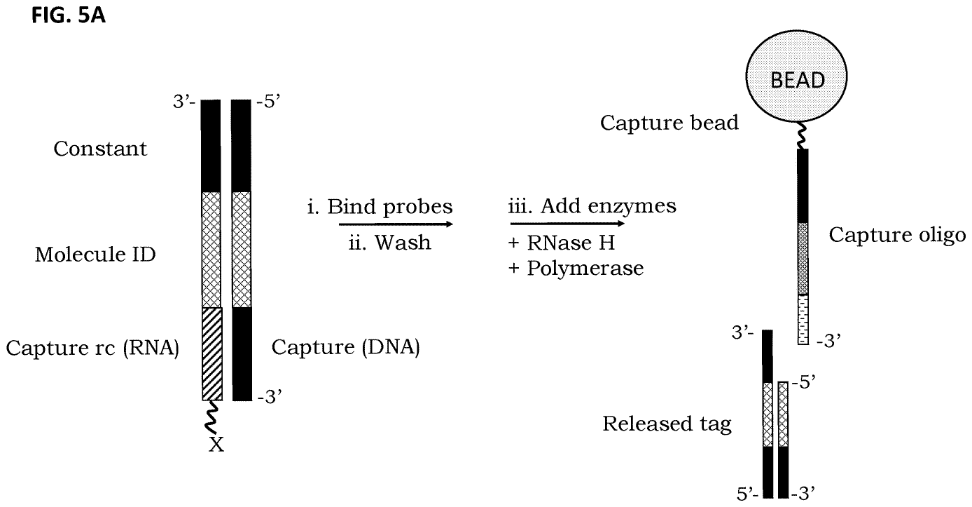

Methods to control or have influence over the direction of the extension step include: 1) block the 3′-end of the capture oligo to prevent extension of the CO (see for instance, the illustration in FIG. 4B), which allows extension of only the tags (which is illustrated herein); or 2) block the 3′-end of the tag, such that when released as a hairpin, it is not extendable, which will allow for extension of only the CO. For the illustrated product release step, methods rely on generating an extension product that creates a DNA/RNA hybrid region by installing RNA bases in the CO, and therefore the release step optionally is not a part of the isothermal reaction. One option described herein is to photo-release these, which is illustrated herein.

In RNA capture embodiments (with amplification by T7 promotor regions), extension occurs by reverse transcription, which would extend the CO and not the 3′-end of the captured RNA. Although that is not a hairpin format, and is actually a captured RNA, the direction of extension occurs from the 3′-end of the CO.

The figures provided herewith show representative overall workflow schemes, including components: encoding and decoding of beads (for instance, using visual molecular barcodes such as those described in WO 2022/187719, and the like) bearing spatially-defined barcodes to generate a spatial encoding array; the probe tag composition (RNA/DNA hybrid hairpin) which enables the isothermal information transfer step; production and cleavage of a prototype tag; the one pot reaction showing cleavage of the tag from a surface (bead), capture to another surface (bead) bearing the capture oligo, which leads to extension and release of the full-length extension product from the capture bead into the supernatant.

Two exceptional elements of herein described workflows are the “sticker” (generally, a spatially-encoded, thin (<250 μm), pliable, 3D macromolecule-permeable matrix, containing a 2D array of capture features at predetermined or “addressed” locations) and the DNA/RNA hybrid tag design. When combined, along with a spatial encoding system (such as the spatial encoding beads described in WO 2022/187719), the workflow provides one-step isothermal cleavage, extension, and release in the sandwich. This workflow is very user-friendly. This platform requires no customized instrumentation, because the pre-reading of the spatial bead array can be performed off-line. Due to the nature of the visual barcodes, locations cannot be re-determined by a user after they are read (reading them destroys the visual barcodes). In embodiments, the consumer receives the spatially encoded bead array, and uses it in combination with, for instance, reagents in a kit format for spatially-resolved biomarker expression in tissue samples. That array is a consumable product that can only be used once.

Also provided are spatially encoded bead arrays such as those described herein, and their uses.

There are multiple other applications for the spatially-encoded bead capture array; this will be the current cheapest way to make spatially encoded bead arrays (to read bead locations by sequencing, this is much more expensive, or to make a microarray to encode locations), whereby each bead has a NGS capture oligo containing a spatial barcode that corresponds (is operationally coupled) to the visual bead barcode on the bead.

Provided herein are Hairpin Tag nucleic acid molecules that include, functionally connected in 5′ to 3′ order, parts A-B-C-D-E, wherein: part A includes a Cleavable Site including either: (1) a string of RNA bases, or (2) a string of DNA bases including a restriction enzyme (RE) recognition site; part B includes a string of DNA bases including a Tag PCR Handle; part C includes a string of DNA bases including a Tag ID Barcode, and which string of DNA bases forms a portion of the loop of the hairpin; part D includes a string of DNA bases having the reverse complementary sequence of the Tag PCR Handle, thereby forming a portion of the stem of the hairpin; and part E includes a string of DNA bases having the reverse complementary sequence of at least a portion of either (1) the string of RNA bases of part A or (2) the string of DNA bases including the RE recognition site, thereby forming a portion of the stem of the hairpin.

Optionally, such Hairpin Tag nucleic acid molecules may further include one or more of: an attachment moiety conjugated to the 5′ end of part A (which attachment moiety in embodiments provides amine-reactive crosslinker activity or thiol-reactive crosslinker activity; a linker between the attachment moiety and part A (which linker in embodiments includes PEG(n), where n=1-20); a Tag ID Barcode that is at least 4 bases long, or in embodiments the Tag ID Barcode is 4, 5, 6, 7, 8, or more than 8 bases long; and/or a Tag PCR Handle in part B and the reverse complement thereof in part D that are each at least 10 bases long (in embodiments, where the Tag PCR Handle in part B and the reverse complement thereof in part D are each 11, 12, 13, 14, 15, 16, 17, 18, 19, 20, or more than 20 bases long.

Hairpin Tag nucleic acid molecules as provided herein optionally may be constructed at least in part using a templated ligation reaction.

In representative Hairpin Tag nucleic acid molecule embodiments, part A includes the string of DNA bases including the RE recognition site, and RE recognition site is at least 4 bases long. For instance, the RE recognition site can be 4, 5, 6, 7, 8, 9, 10, 11, 12, 13, 14, or longer than 14 bases long. Optionally, the RE recognition site within any Hairpin Tag nucleic acid molecule may be a Type IIS Restriction Enzyme recognition site, a site for a RE that produces a 3′ overhang, or both.

Additional representative Hairpin Tag nucleic acid molecules are DNA/RNA Hybrid Hairpin Tag nucleic acid molecules, wherein part A includes a single RNA, or a string of RNA bases, which string of RNA bases in part A is at least 5 bases long. Thus there are contemplated DNA/RNA Hybrid Hairpin Tag nucleic acid molecules, wherein the string of RNA bases in part A is 5, 6, 7, 8, 9, 10, 11, 12, 13, 14, 15, 16, 17, 18, 19, 20, or longer than 20 bases long.

Also provided are sets of two or more Hairpin Tag nucleic acid molecules as described and/or DNA/RNA Hybrid Hairpin Tag nucleic acid molecules as described, where each of the two or more nucleic acid molecules has a unique Tag ID Barcode sequence. In examples of such sets, the set further includes at least one Attenuation Tag-like nucleic acid molecule, which Attenuation Tag-like nucleic acid molecule differs from a Hairpin Tag nucleic acid molecule or DNA/RNA Hybrid Hairpin Tag nucleic acid molecule of the set by lack of a functional Cleavable Site. By way of example, there are provided sets of two or more Hairpin Tag nucleic acid molecules and/or DNA/RNA Hybrid Hairpin Tag nucleic acid molecules, wherein the Attenuation Tag-like nucleic acid molecule differs from a Hairpin Tag nucleic acid molecule or DNA/RNA Hybrid Hairpin Tag nucleic acid molecules of the set by (1) having DNA bases in place of the RNA bases of part A, or (2) lacks the RE recognition site of part A.

Also provided are Tagged Probes, which including a Probe Molecule to which is attached through the attachment moiety to a Hairpin Tag nucleic acid molecule as described, or to a DNA/RNA Hybrid Hairpin Tag nucleic acid molecule as described. By way of example, the Probe Molecule includes an affinity molecule having a binding affinity for a target molecule. Optionally, the affinity molecule includes an antibody binding domain having affinity for an antigen, and the target molecule includes the antigen.

Also provided are Tagged Probes, wherein the probe molecule includes one or more of: an antibody or binding fragment thereof, a nucleic acid, a small molecule, an organic or inorganic chemical, a putative drug target, an identified pharmaceutical drug, or a biological macromolecular complex. For instance, the probe molecule can be one of a set of probe molecules, each of which includes one of a plurality of members of a library of small molecules, a library of drug targets, a library of biological affinity molecules, a library of natural products, a library of bio-active compounds, a genomic library, a transcriptomic library, a metabolomic library, or a drug screening library.

In yet other embodiments of Tagged Probes, the target molecule includes a biological molecule, an inorganic object, or an addressable feature of an array. For instance, the target molecule can include a biological molecule, and the biological molecule includes one or more of a protein, lipid, carbohydrate, a nucleic acid molecule, or a combination of proteins, lipids, carbohydrates and/or nucleic acid molecules. Optionally, the target molecule in some cases is one of a plurality of molecules making up a complex, and the complex is located outside of or within a cell or cells in a tissue sample.

In any Tagged Probe embodiments, the tagged probe can further include an amplification sequence including a polymerase promoter sequence. By way of example, the amplification sequence includes a T7 promoter sequence, such as a T7 promoter adaptor.

Another embodiment is a Released Hairpin Tag nucleic acid molecule derived from a Hairpin Tag as described, or from a DNA/RNA Hybrid Hairpin Tag as described, wherein the Released Hairpin Tag has been separated from the attachment moiety by enzymatic action of a Restriction Endonuclease or a RNase H enzyme.

Yet another embodiment is a Capture Oligo (CO) nucleic acid molecule that includes, functionally connected in 5′ to 3′ order, parts I-II-III-IV, wherein: part I includes a Cleavable Site including either: (1) a single RNA base or a contiguous string of RNA bases, or (2) a string of DNA bases including a restriction enzyme (RE) recognition site; part II includes a string of DNA bases including a CO PCR Handle; part III includes a string of DNA bases including a Spatial Barcode; and part IV includes a string of DNA bases including a Tag Capture Region. Optionally, the CO nucleic acid molecule further includes an attachment moiety conjugated to the 5′ end of part I. For instance, the attachment moiety in instances provides amine-reactive crosslinker activity or thiol-reactive crosslinker activity.

In further options, the CO nucleic acid molecule includes a linker between the attachment moiety and part 1. For instance, example linkers include PEG(n), where n=1-20.

In further CO nucleic acid molecule embodiments, the Spatial Barcode is at least 4 bases long. For instance, the Spatial Barcode can be 4, 5, 6, 7, 8, or more than 8 bases long.

Optionally, in embodiments of the CO nucleic acid molecules, the CO PCR Handle in part II is at least 5 bases long. For instance, the CO PCR Handle in part II can be 5, 6, 7, 8, 9, 10, 11, 12, 13, 14, 15, 16, 17, 18, 19, 20, or longer than 20 bases long.

Also contemplated are CO nucleic acid molecules that further include a unique molecular identifier (UMI).

In any of the CO nucleic acid molecule embodiments, examples are contemplated in which the sequence has no more than 2 contiguous bases of internal sequence self-complementarity. By way of example, the sequence of the CO nucleic acid molecule has no more than more than 3, no more than 4, no more than 5, no more than 6, no more than 7, no more than 8, no more than 9, no more than 10, no more than 11, or no more than 12 contiguous bases of internal sequence self-complementarity.

In any of the CO nucleic acid molecule embodiments, examples are constructed at least in part using a templated ligation reaction.

In any of the CO nucleic acid molecule embodiments, optionally part I includes the string of DNA bases including the RE recognition site, and RE recognition site is at least 4 bases long. for instance, the RE recognition site can be 4, 5, 6, 7, 8, 9, 10, 11, 12, 13, 14, or longer than 14 bases long. Optionally, the RE recognition site is a Type IIS Restriction Enzyme recognition site, a site for a RE that leaves a 3′ overhang, or both.

Also contemplated are CO nucleic acid molecules that are a DNA/RNA chimeric CO nucleic acid molecule, and wherein part I includes a single RNA base. Also provided are DNA/RNA chimeric CO nucleic acid molecule, wherein part I includes the contiguous string of RNA bases. By way of example, such DNA/RNA chimeric CO nucleic acid molecules can contain a contiguous string of RNA bases in part I is 2, 3, 4, 5, 6, 7, 8, 9, 10, 11, 12, 13, 14, 15, 16, 17, 18, 19, 20 bases long, or longer than 20 bases long.

Yet another embodiment is a set of two or more CO nucleic acid molecules of any of the provided CO nucleic acid molecules, and/or DNA/RNA chimeric CO nucleic acid molecules of any the provided DNA/RNA chimeric CO nucleic acid molecules, where each of the two or more nucleic acid molecules has a unique Spatial Barcode sequence.

Also provide are CO nucleic acid molecules or DNA/RNA chimeric CO nucleic acid molecules, each of which is attached to a Capture Feature through the attachment moiety of nucleic acid molecule. By way of example, the Capture Feature in various embodiments is a bead, a chemically-functionalized spot on a glass surface, a defined region of a chemically functionalized and permeable gel, a bead or other inorganic object embedded within or on the surface of a permeable gel, or one of a series of spatially defined objects attached to a gel. Also contemplated are attached CO nucleic acid molecules, wherein the Capture Feature is a spatially addressable feature in an array, such as a microarray, for instance a microarray having at least 100 addressable Capture Features.

Yet another provided embodiment is a Spatially Encoded Capture Feature, which include a Capture Feature to which is attached through the attachment moiety a CO nucleic acid molecule as provided or a DNA/RNA chimeric CO nucleic acid molecule as provided. By way of example, the Capture Feature includes a bead or an addressable location on a substantially 2-dimensional surface. For instance, representative Spatially Encoded Capture Feature embodiments include one Spatially Encoded Capture Feature within an array of at least 100 different Spatially Encoded Capture Features, and wherein the CO nucleic acid molecule on each of the at least 100 different Spatially Encoded Capture Features of the array each include a different Spatial Barcode.

Also described are Capture Pairs, which including: a Hairpin Tag nucleic acid molecule as provided, or a DNA/RNA Hybrid Hairpin Tag nucleic acid molecule as provided, or a Released hairpin tag nucleic acid molecule as provided; and a Capture Oligo (CO) nucleic acid molecule of as provided or a DNA/RNA chimeric CO nucleic acid molecule as provided, or an Attached CO as provided, wherein the sequence of the Hairpin Tag nucleic acid molecule and the sequence of the CO nucleic acid molecule are at least partially complementary, such that when the Hairpin Tag nucleic acid molecule is released from its attachment moiety in proximity to the CO nucleic acid molecule, the released Hairpin Tag nucleic acid molecule is captured by sequence complementarity bonding at the 3′ ends to the CO nucleic acid molecule, such that resultant complex of the released Hairpin Tag nucleic acid molecule and the CO nucleic acid molecule is competent for a downstream extension reaction by a polymerase enzyme.

Yet another embodiment is a Spatially Encoding Capture Array, which includes a defined array of spatially-addressed capture features, wherein each capture feature includes: a spatially-identifiable feature including: a pre-defined, addressable location on a substantially two-dimensional solid surface; or a bead or other similar separate, solid capture object; and attached at each feature, multiple copies of a CO nucleic acid molecule as provided or a DNA/RNA chimeric CO nucleic acid molecule as provided, wherein the CO nucleic acid molecules at each feature have a unique Spatial Barcode sequence compared to the CO nucleic acid molecules at other features in the array. In examples, one or more CO nucleic acid molecule(s) is applied to spatially-addressed capture feature(s) in the array by: droplet printing of the CO into the pre-defined, addressable locations on the substantially two-dimensional solid surface, or attachment of the CO onto beads through the attachment moiety.

Optionally, the Spatially Encoding Capture Array includes beads each of which include a Visual Barcode operationally coupled to the COs. For instance, examples of the Spatially Encoding Capture Array include Visual Barcode that enables beads to be assigned to locations within in the capture array.

Also contemplated are examples of Spatially Encoding Capture Arrays, wherein the bead or other similar, separate capture objects are embedded in a biomolecule-permeable matrix. For instance, the capture objects can include beads; the biomolecule-permeable matrix includes a gel; or both. In specific examples, the biomolecule-permeable matrix that includes the gel is formatted as a pliable sticker.

Additional example Spatially Encoding Capture Arrays include the biomolecule-permeable matrix: is structurally stable at a selected temperature between 4-45° C.; is permeable to proteins such as functional RNAseH and polymerase; is permeable to ribonucleoside tri-phosphates (rNTPs) and/or deoxynucleotide triphosphates (dNTPs); is permeable to Mg²⁺ ions; is substantially inert to biological molecules; and is sufficiently flexible to permit application of a relatively thin layer of the matrix directly to a substantially two-dimensional sample or surface. For instance, the Spatially Encoding Capture Array may be formulated as a three-dimensional thin-layer gel, the width and length of which are substantially larger than its thickness, and wherein the spatially-identifiable capture features are arranged substantially in a single plane across surface of the gel defined by its the length and width. In instances, at least a first of the spatially-identifiable capture features is immediately adjunct and/or touching a second of the spatially-identifiable capture features.

In any of the provided Spatially Encoding Capture Arrays, the matrix can include a hydrogel or polyacrylamide gel. By way of example, the thickness of the matrix or gel is no more than about 2 mm. In specific instances, the thickness of the matrix or gel is no more than about 1 mm, no more than 500 μm, no more than 250 μm, no more than 200 μm, no more than 150 μm, no more than 125 μm, no more than 100 μm, or less than 100 μm. Beneficially, the thickness of the matrix or gel in instances is 100-200 μm, 100-150 μm, or about 125 μm.

In any instance of provided Spatially Encoding Capture Arrays, it is contemplated that the array can be reinforced by an inert mesh or other support structure.

In examples of provided Spatially Encoding Capture Arrays, the pre-defined, addressable location on a substantially two-dimensional solid surface has a surface area of no more than about 1 μm×1 μm; or the bead or other similar separate, solid capture object has diameter of no more than about 20 μm.

In examples of provided Spatially Encoding Capture Arrays, the bead or other similar separate, solid capture object has diameter of no more than 18 μm, no more than 15 μm, no more than 12 μm, no more than 10 μm, no more than 8 μm, no more than 5 μm, no more than 3 μm, no more than 1 μm, or about 100 nm. For instance, the bead or other similar separate, solid capture object may have a diameter of between 1-3 μm.

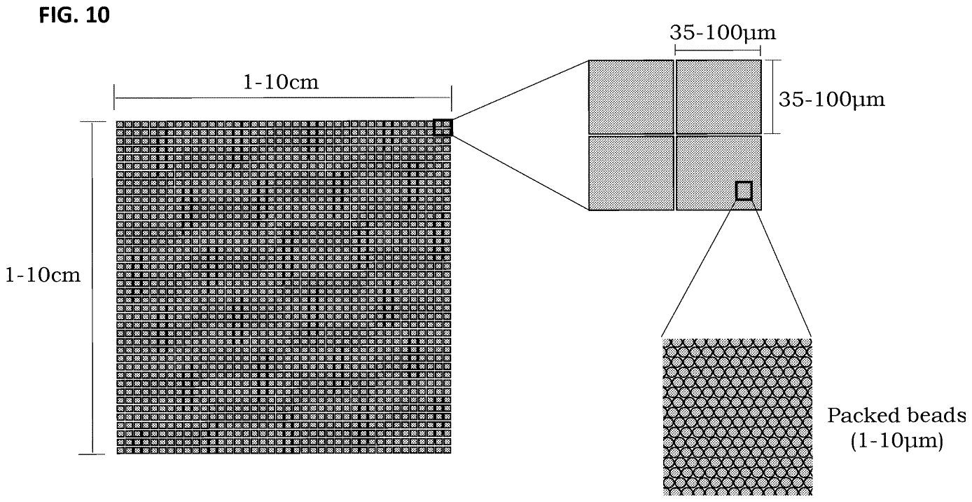

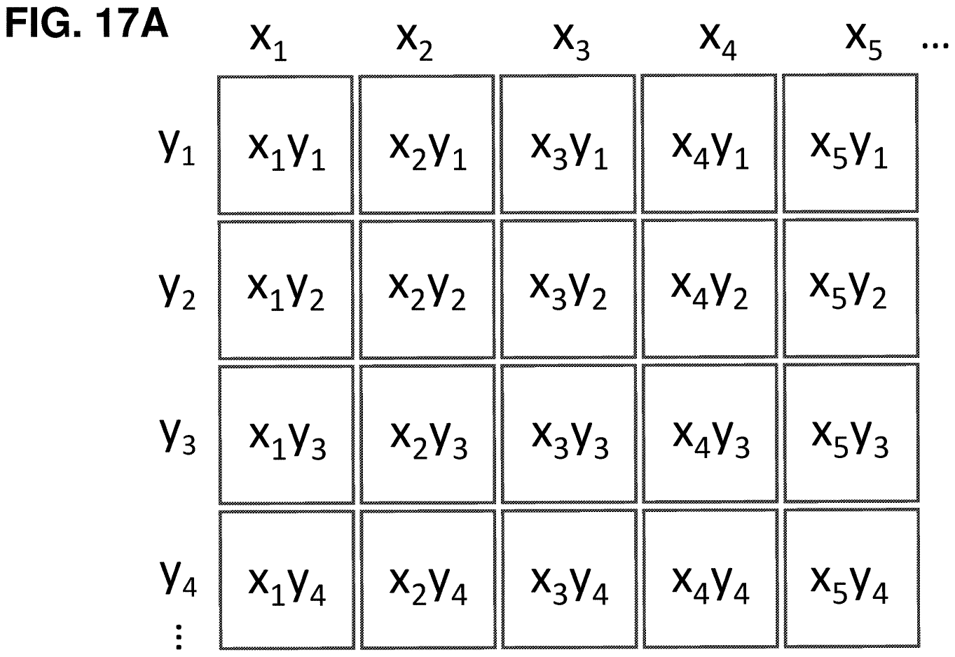

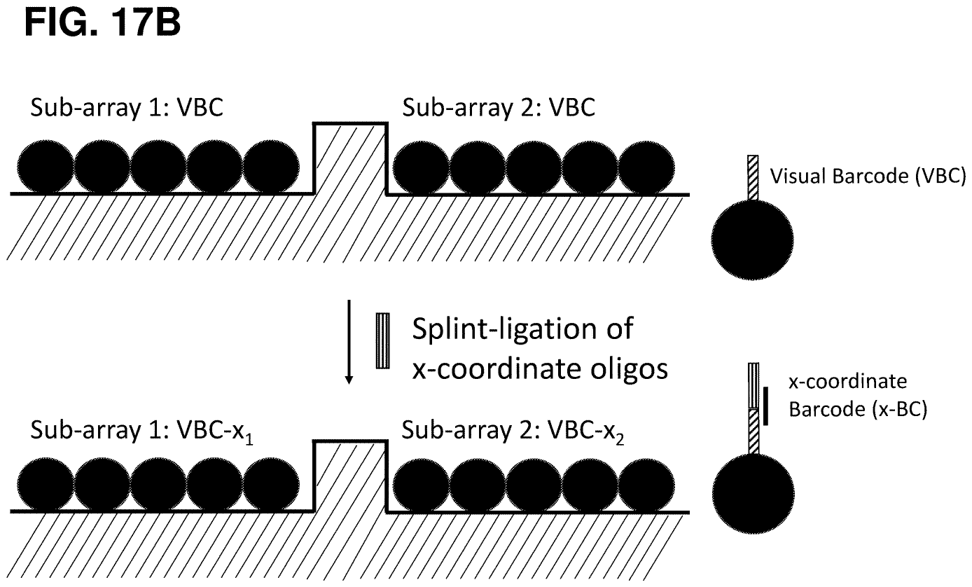

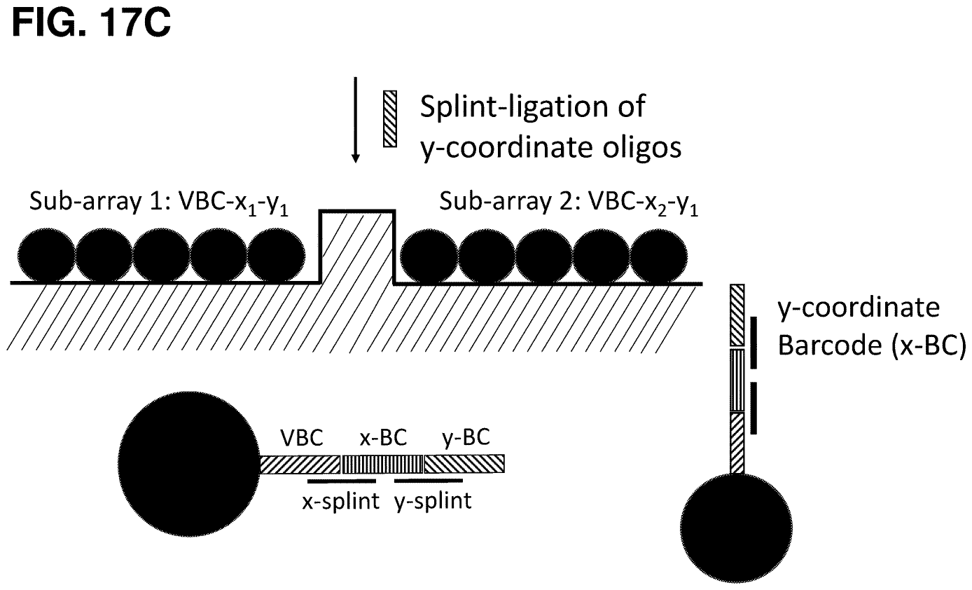

Another embodiment is a semi-ordered Spatially Encoding Capture Array, which includes: a grid of spatially addressable locations, each of which is labeled with oligonucleotides having a unique X-Y coordinated sequence, which oligonucleotides are applied to the spatially addressable locations by splint ligation of: a x-coordinate adapter oligonucleotide, which x-coordinate adapter oligonucleotide is used to label all spatially addressable locations within a row of the grid; and a y-coordinate adapter oligonucleotide, which y-coordinate adapter oligonucleotide is used to label all spatially addressable locations within a column of the grid. Examples of such semi-ordered Spatially Encoding Capture Arrays are constructed at least in part using a method provided in FIGS. 17A-17C. In embodiments, the semi-ordered Spatially Encoding Capture Array of has a grid-within-a-Grid format as illustrated in FIG. 10.

Also provided are semi-ordered Spatially Encoding Capture Arrays, that include: an array of uniquely identifiable capture features, which array includes two or more sub-arrays, each sub-array including uniquely identifiable capture features the location of which is specified at least in part by identification of the sub-array within the semi-ordered Spatially Encoding Capture Array.

Another example is a semi-ordered Spatially Encoding Capture Array, that includes: a set of two or more sub-arrays each including a plurality of capture features, in which each capture feature within each sub-array is attached to a capture oligo including a unique Location Tag, the capture features within each sub-array are randomly arranged, and the capture features within each sub-array further include a sub-array-identifying oligo tag attached by splint ligation to the capture oligos on each feature in the sub-array.

Also provided are methods for detection and/or quantification and/or localization of targets in a substantially two-dimensional (2D) sample, which methods involve: contacting a substantially 2D sample with at least one tagged probe to produce a substantially 2D stained sample, which tagged probe includes: a Hairpin Tag nucleic acid molecule including an attachment moiety and a Tag ID Barcode, or a DNA/RNA Hybrid Hairpin Tag nucleic acid molecule including an attachment moiety and a Tag ID Barcode; and a Probe Molecule attached through the attachment moiety to the Hairpin Tag nucleic acid molecule including a Tag ID Barcode, or the DNA/RNA Hybrid Hairpin Tag nucleic acid molecule including a Tag ID Barcode; contacting a surface of the substantially 2D stained sample with a permeable, spatially encoding capture array to form a sample-array sandwich, which spatially encoding capture array includes: a plurality of spatially identifiable features; and attached at each spatially identifiable feature, multiple copies of a Capture Oligo (CO) nucleic acid molecule or a DNA/RNA chimeric CO nucleic acid molecule, wherein the CO nucleic acid molecules at each feature have a unique Spatial Barcode sequence compared to the CO nucleic acid molecules at other features in the array; placing a flow cell or other solution-containing cover over the sample-array sandwich to form an enclosure containing the sample-array sandwich; adding to the enclosure a solution including reaction components to form a reaction mixture, which components include: a cleavage enzyme selected from a RNAseH or at least one restriction endonuclease (RE); at least one polymerase; a mixture of ribonucleoside tri-phosphates (rNTPs) and/or deoxynucleotide triphosphates (dNTPs); Mg²⁺ ions; and a buffering agent; incubating the sample-array sandwich in contact with the reaction mixture at an assay temperature for 30-60 minutes, to form a reaction product mixture; removing at least a portion of the reaction product mixture from the enclosure; and analyzing the reaction product mixture to detect and/or quantify and/or define the location of targets in the substantially 2D sample. Optionally, in such methods the enclosure includes a flow cell. Embodiment of the provided method provide location information for more than one target within the substantially 2D sample.

Embodiments of the methods are carried out at a single temperature (isothermally) or within a range of about 5° C. within a single temperature. By way of example, the assay temperature has a range of 20-55° C., or 37-42° C.

By way of example, in any one of method embodiments, the at least one polymerase provides an enzymatic activity of DNA polymerase, reverse transcriptase, or RNA polymerase.

By way of example, in any of the method embodiments, analyzing the reaction product mixture includes sequence analysis of a plurality of nucleic acid molecules containing a Spatial Barcode and a Tag ID Barcode. For instance, analyzing the reaction product mixture in some cases includes next generation sequencing (NGS) of a plurality of nucleic acid molecules containing a Spatial Barcode and a Tag ID Barcode. For instance, the plurality of nucleic acid molecules containing a Spatial Barcode and a Tag ID Barcode in some methods are full extension products released from the spatially identifiable features by cleavage (e.g., cleavage based on RNAseH enzymatic activity or restriction endonuclease activity) at the Cleavage Site of the CO. In examples of the methods, analyzing includes assigning a spatial location of at least one nucleic acid molecule containing a Spatial Barcode and a Tag ID Barcode within the substantially 2D sample.

Also provided are methods for isothermal spatial encoding of a biological sample, including the method of detection and/or quantification and/or localization of targets in a substantially two-dimensional (2D) sample, wherein the substantially 2D sample is a biological sample.

Another embodiment is use of a set of Hairpin Tag nucleic acid molecules as provided herein, or a set of Capture Oligo (CO) nucleic acid molecules of provided herein, or both, for transcriptomic analysis of a biological sample.

Also described are spatial encoding surfaces, such as a capture array, for instance a bead-based capture array, substantially as described or illustrated herein.

Yet another embodiment is a spatial encoding workflow, including: contacting a hybrid RNA/DNA tag including a spatial barcode conjugated to an antibody probe with a substantially two-dimensional (2D) tissue sample, to produce a stained sample; placing a capture array including capture features in contact with the stained sample to produce a sample/array sandwich; placing a fluid-containment enclosure on top of the sample/array sandwich; introducing assay solution including active RNAseH and active polymerase into the fluid-containment enclosure, thereby bringing the assay solution into contact with the sample/array sandwich; incubating the sample/array sandwich in the sample solution at a temperature and for a period of time sufficient for the RNAseH activity to at least partially digest the hybrid RNA/DNA tag to produce a cleaved tag and thereby releasing the cleaved tag into the assay solution in proximity to a capture feature; permitting interaction of the cleaved tag with a capture oligo (CO) on the proximal capture feature to provide a captured cleaved tag; incubating the sample/array sandwich in the sample solution at a temperature and for a period of time sufficient for the polymerase activity to extend the captured cleaved tag using the CO as template, to produce an extension product; cleaving the extension product, upon sufficient extension to produce a complementary RNA/DNA region based on the RNA base(s) in the CO, with RNAseH in the sample solution, thereby releasing the full-length extension products; collecting at least a portion of the full-length, released extension products; and amplifying and/or sequencing at least one of the full-length, released extension products.

Also provided are computer readable medium or digital resources, and digital databases, containing spatial location information for features of a spatially encoding array as described herein. By way of example, the computer readable medium or digital resource, or a digital database in some instances contains spatial location information for substantially all the features of the spatially encoding array.

Use of the provided computer readable medium or digital resource, or a digital database, to provide a user with location information for one or more targets correlated with the spatial location information of the spatially encoding array, is also disclosed. By way of example uses of the provided computer readable medium or digital resource, or a digital database, the correlation arises through use of the spatially encoding array in a workflow or method as described or illustrated herein.

Yet another embodiment is a kit useful for carrying out one of the methods provided herein. Representative kits include one or more of: two or more Hairpin Tag nucleic acid molecules as described; two or more DNA/RNA Hybrid Hairpin Tag nucleic acid molecules as described; a described set of two or more Hairpin Tags; a set of two or more Tagged Probes of as described; two or more Capture Oligo (CO) nucleic acid molecules as described; two or more DNA/RNA chimeric CO nucleic acid molecules as described; a described set of two or more CO nucleic acid molecules; a described set of two or more DNA/RNA chimeric CO nucleic acid molecules; two or more CO nucleic acid molecules, each attached to a capture feature, as described; at least one spatially encoding capture array as described; at least one semi-ordered spatially encoding capture array as described; or a spatial encoding surface as described.

In examples of such kits, the spatially encoding capture array, semi-ordered spatially encoding capture array, or spatial encoding surface is in the format of a pliable sticker.

Also provide are kit embodiment, further including one or more of: a container in which is contained functional RNAseH enzyme; a container in which is contained functional polymerase enzyme; a container in which is contained functional restriction enzyme; a container in which is contained one or a mixture of ribonucleoside tri-phosphates (rNTPs) and/or deoxynucleotide triphosphates (dNTPs); a container in which is contained a solution including Mg²⁺ ions; or a container in which is contained a buffer solution. Optionally, in such kits, wherein at least one of the containers contains at least two of the RNAseH enzyme, the polymerase enzyme, the restriction endonuclease enzyme, the rNTPs and/or dNTPs, the Mg²⁺ ions, or the buffer solution. Optionally, a complete assay solution is provided in a single container.

Kit embodiments may also, optionally, include one or more of: components useful for preparing a sample for analysis using a method provided herein; a solution-containing cover suitable for placement over a sample-array sandwich on a slide, in order to form a fluid-containing enclosure for the sample-array sandwich; a flow cell cover; or a glass slide or other surface suitable for receiving a substantially two-dimensional sample.

Aspects of the current disclosure are now described with additional details and options.

### Methods and Systems for Isothermal Spatial Encoding of Biological Samples and Assays

Isothermal method: Isothermal information transfer, spatial encoding, and spatially-encoded product release into solution. As described herein, a new type of nucleic acid tag is used for encoding molecules or probes, which tag includes a hairpin structure whereby a chimeric RNA/DNA molecule generates a region of RNA/DNA hybridization through base complementarity. This unit is very stable, resistant to RNA exonuclease activity, and can contain a specific barcode within the loop region of the hairpin (embodiments with a four-base stretch of contiguous, unpaired bases are provided as examples, though longer lengths are contemplated) to distinguish different probes from each other in multiplex assays. Also contained within the sequence of the hairpin tag is the first of two PCR handles used for downstream amplification of spatially-encoded products. Methods for constructing different versions of this hairpin tag are provided, and their utility in workflows when conjugated to antibody probes (for instance) is demonstrated.

After binding probes conjugated to RNA/DNA hybrid-containing hairpin tags to a surface, and washing away unbound conjugates, the hairpin tag can be released as a smaller, all DNA hairpin (a released tag) from the probes while bound to the sample surface by the use of an enzyme containing RNAse H activity. In embodiments, prior to their release by RNAse H, a spatially-encoded capture array is placed in proximity (for instance, directly in contact with) the sample surface containing the bound probe conjugates. As described herein, this capture array surface in embodiments is an array of 1 μm beads coated with capture oligos. Also demonstrated here, this capture surface may include an enzyme-permeable matrix (e.g., gel) in which the capture beads are embedded. This may be in the form of a sticker, which comprises the capture beads in the matrix, that is sufficiently flexible to permit it to be applied in direct contact with the sample surface being assayed.

Also contemplated are embodiments in which many different tag barcodes are included on the same antibody, such that it is possible to analyze the normal the distribution of the tags after amplification (for instance, where they are all released from the same antibody type, and should be in equimolar ratios). This embodiment is generally outside of normal workflows, but can be used for instance as an internal control or calibration.

In embodiments, capture oligos are bound to capture features (such as beads) through their 5′-ends, and contain a 3′ capture region designed for specific base complementarity with 3′-ends of released DNA hairpins, as well as a locational barcode, and the second of two PCR handles used for downstream amplification of spatially-encoded products. Capture oligos may also contain a 5′ stretch of RNA bases placed between the second PCR handle and the 5′ bead attachment moiety.

Locations of beads/features within the capture array are pre-determined, and this bead location information is contained within the sequence of the capture oligos (spatial or locational barcode region) bound to the capture beads or feature. Each capture bead or feature contains a unique spatial barcode, which is common to all capture oligos on that bead or within that feature address. Locations of beads or features within the capture array may be uploaded and stored in a software package or database, for instance in the form of DNA sequences (the locational or spatial barcode) that correspond to the specific location of each bead or feature.

Once placed in proximity, and upon introduction of RNAse H, information from the sample (released hairpins from probes) is transferred to (the most proximal) spatially-encoded capture beads or features via specific hybridization with capture regions contained within the 3′ sequence of capture oligos bound to capture beads. Within the same reaction mixture is a polymerase enzyme and necessary cofactors (Mg²⁺, dNTPs) for extension of hairpin 3′-ends by the DNA polymerase, as templated by the capture oligo, which copies the location information and second PCR handle onto the released hairpin oligo. 5′ RNA bases can optionally be placed within the capture oligos to create a second substrate for recognition and cleavage by RNAse H activity present in the isothermal one-pot reaction mixture. Inclusion of RNA bases at the 5′-end of the capture oligos enables the spatially-encoded probes to release from the capture beads and into the surrounding solution.

When using a capture array in which capture oligos (or beads) are embedded within a macromolecule-permeable surface or gel, the capture surface can be placed in proximity with the probe-bound sample prior to the introduction of enzymes and other reaction components, which enzymes/components can diffuse through the permeable surface/gel. A flexible encoding surface can be beneficial to conform to the sample surface, which places the spatially encoded capture features (beads as illustrated in embodiments herein) in close proximity with the sample. The provided “one-pot” isothermal encoding reaction can then be initiated by soaking the one-pot encoding reaction solution through the gel while incubating at the reaction temperature (e.g., 37-42° C.). In the reaction, information from the sample is transferred via RNAse H digestion of hairpin tags followed by capture of the released hairpin tags in proximity to where they were bound to the sample, then spatially-encoded via an extension reaction by a polymerase, then optionally released into the surrounding solution following spatial encoding. Application of the encoding, macromolecular-permeable capture surface or gel directly onto the sample allows for one or more of the enzymes (such as at least the triggering enzyme, E. coli RNAse H, as demonstrated herein) and/or cofactors required for the isothermal reaction(s) to diffuse through the encoding (spatially-identifying) surface and into contact with appropriate substrate(s) without moving the capture surface relative to the sample.

Following the reaction, spatially-encoded reaction products diffuse out of the gel (or other permeable matrix) and into the surrounding solution where they can be retrieved by aspiration of the solution. There needs to be sufficient volume of solution to cover the spatial encoding gel (capture array), but there optionally could be a flow cell covering the entire area of the spatial encoding gel, which would then be what defines the volume of solution. Ideally, this is a low volume of solution, so that the concentration of the diffused products is kept high. However, the volume is of less importance in embodiments that employ a described optional capture step(s).

In an exemplified embodiment, the solution volume is ~40 μl. If that volume is significantly higher, an additional (bead) capture step can be implemented in order to capture the diffused spatially encoded products—for instance onto beads. That can be accomplished by the fact that spatially encoded products can be released from capture beads following the extension step which generates a new DNA/RNA hybrid region. The region of DNA that becomes exposed following removal of the RNA region (following extension and exposure to RNAse H activity in the reaction solution) can be captured using beads (or another capture surface) containing immobilized oligos that are designed to contain a complementary region for capture of the extended and released products. Following capture in a 2× capture buffer (added 1:1 by volume to the aspirated solution containing spatially encoded and released extension products) containing 20 mM Tris pH 7.5, and 1M NaCl, an optional wash may be performed in the 1× buffer, then the beads are brought down to a low volume (e.g., by magnet or by centrifugation to pellet the beads), for resuspension in a consistent, low volume (less than 25 μl) of elution solution or buffer containing low to no salt (which could be water, or 10 mM Tris pH 7.5, for instance). The higher salt in the capture step will increase the propensity to capture spatially encoded extension products by hybridization to the bead-immobilized oligos. The low salt in the elution buffer will increase the propensity to de-hybridize captured products from the bead-immobilized oligos when exposed to heat (>75° C.). The low elution volume will provide consistency across experiments and will enable retrieval of all products diffused from the gel during the isothermal reaction.

These eluted products may be further processed, for instance by capture and/or by PCR amplification. These amplified products can then be prepared for next generation sequencing using known amplicon-based or ligation-based sequencing library preparation techniques. See, for instance, information available online at illumina.com/techniques/sequencing/ngs-library-prep.html (which describes multiple types of library preparations for Illumina sequencing, including both amplicon-based preps and ligation-based preparations.) The resultant library of sequencing data contains contiguous reads (counts) that contain individual probe hairpin sequences fused with the locational information that was contained within the capture oligos that were bound to the beads onto which the hairpins were captured. The pre-determined map of bead locations (barcodes) is then used (for instance, through a license or subscription service access) for reconstructing a digital image by overlaying counts for each probe that was obtained by sequencing, onto the spatially-encoded array at the location in which they were captured. This workflow therefore enables digital image reconstruction through the use of next generation sequencing.

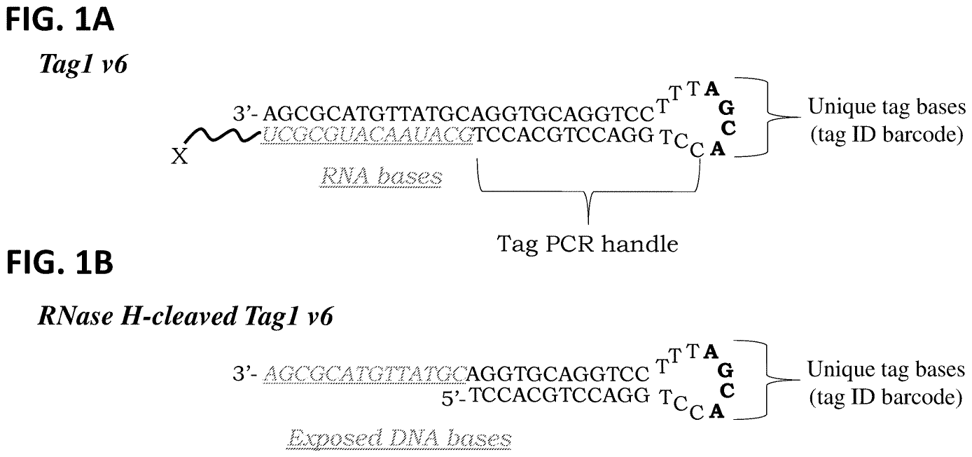

The hairpin nucleic acid tags described herein contain both RNA and DNA, to generate a substrate for cleavage by RNAse H (see FIGS. 1A-1B). Benefits arising from this design include: the ability to reveal a rationally-designed stretch of DNA sequence by RNAse H digestion for optimal capture of the released hairpin at isothermal temperature; the ability to both capture and extend such a sequence onto a capture oligo bound within a capture feature contained within an array of spatially ascribed capture features; optimization of hairpin capture region Tm under isothermal reaction conditions; the method functions without crippling antibody recognition of targets in the analyzed sample.

As described herein, the provided hairpin has been shown to be stable for at least 6 weeks when conjugated to an antibody and stored at 4 degrees in phosphate buffered saline (PBS)+bovine serum albumin (BSA).

Described herein is use of hairpin tags that contain both RNA and DNA for isothermal release of information from a DNA-encoded molecule bound to a surface following a selective binding event. Benefits arising from this include: the ability to “trigger” release of the hairpin upon contact of the tag with an enzyme containing RNAse H activity; and the ability to trigger release of the hairpin at a temperature compatible with capture of the released tag onto a capture oligo.

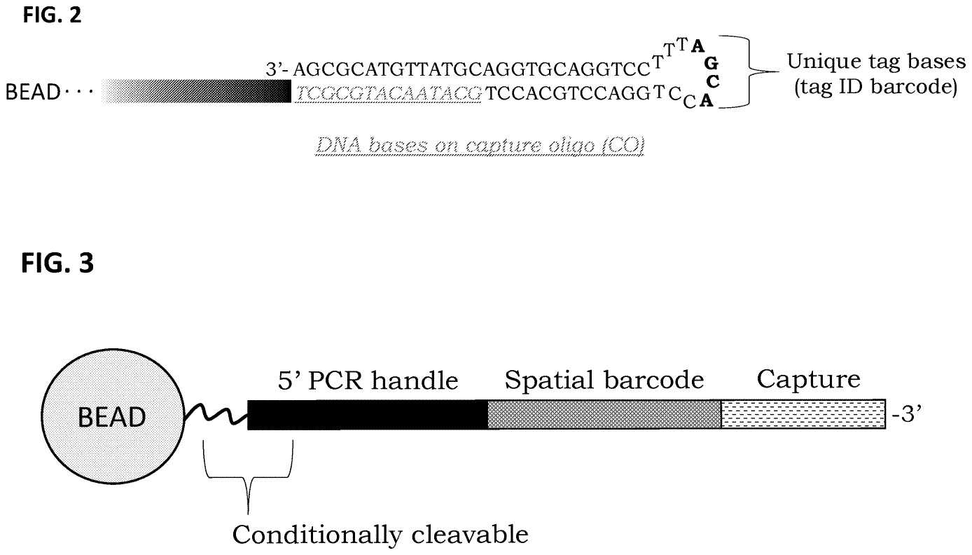

Also provided is capture of information released from a DNA-encoded molecule (FIG. 2) bound to a surface following a selective binding event, by the use of a capture oligo (structure shown in FIG. 3) coupled with an extension reaction performed by a polymerase enzyme that is included within the same reaction mixture (FIG. 4). Benefits arising from this include: the ability to reduce lateral diffusion of probes or “hopping” of probes to different capture oligos because the extension reaction immediately “locks-on” each hairpin to the first (or one of the first) capture oligos it comes in contact with via extension by the polymerase; and the ability to copy location information from highly proximal capture oligos onto released DNA hairpin oligos (and vice versa) within the same reaction mixture in which the hairpin oligos were released from the sample surface by templated extension of 3′-ends of captured oligos (and capture oligos in one version; when 3′-ends of capture oligos are not blocked).

When using a polymerase enzyme that contains reverse transcriptase activity (for instance, RNA-dependent DNA polymerase activity), reading through of RNA bases contained within the capture oligo template during the extension reaction is possible. Here, a polymerase that contains both RNA- and DNA-dependent DNA polymerase activity was used, which enables incorporation of deoxyribonucleotide triphosphate bases (dNTPs) in response to either DNA or RNA bases in the capture oligo template. Data described herein demonstrate: PCR amplification for retrieval of full-length products following hairpin capture and extension on capture beads; qPCR results from +/−experiments; gels of amplified full-length products; and Sanger sequencing traces of full-length extension products prepared for NGS (see Appendix A included in priority U.S. Provisional Application No. 63/487,575, filed on Feb. 28, 2023).

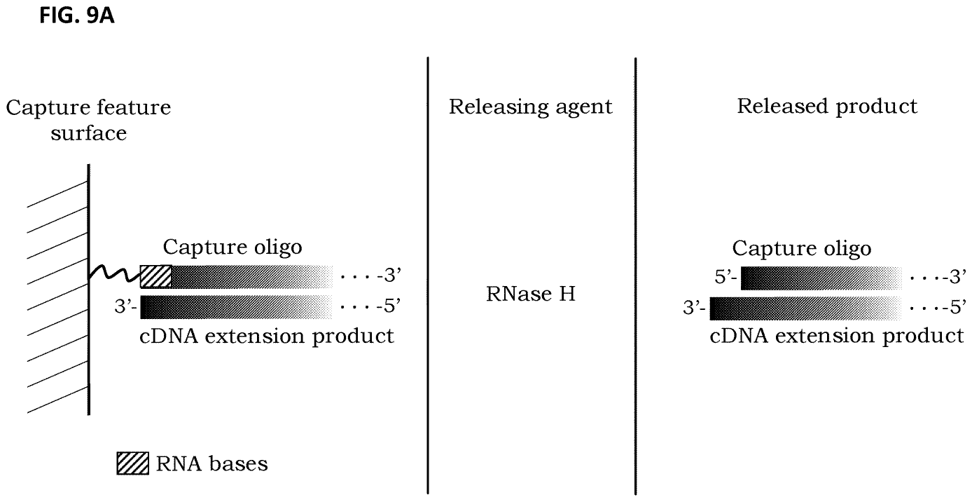

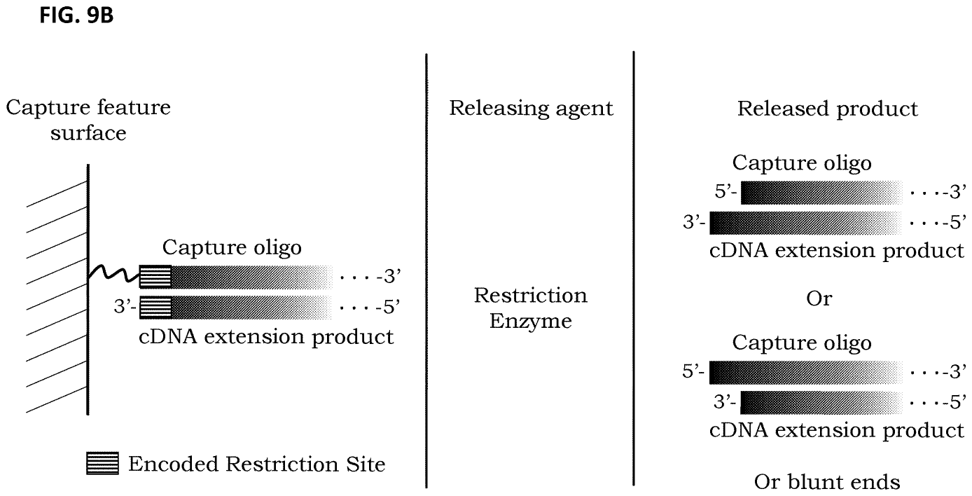

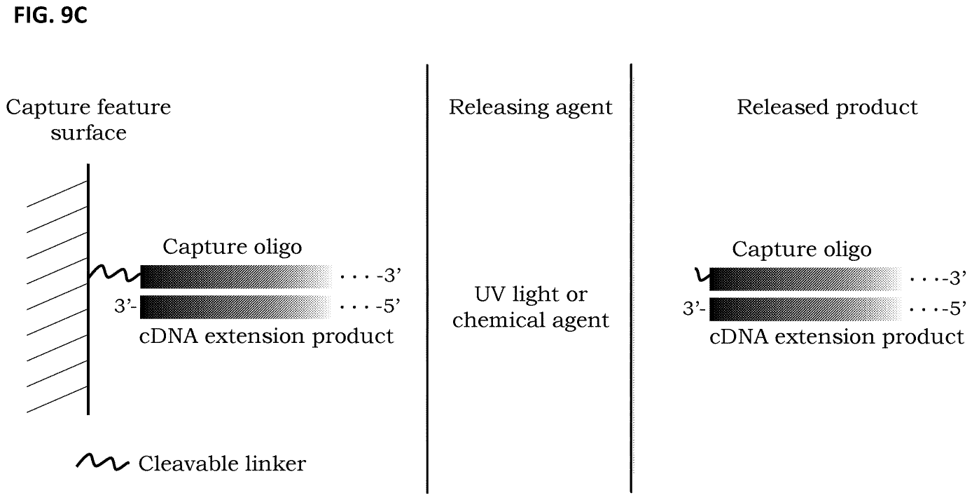

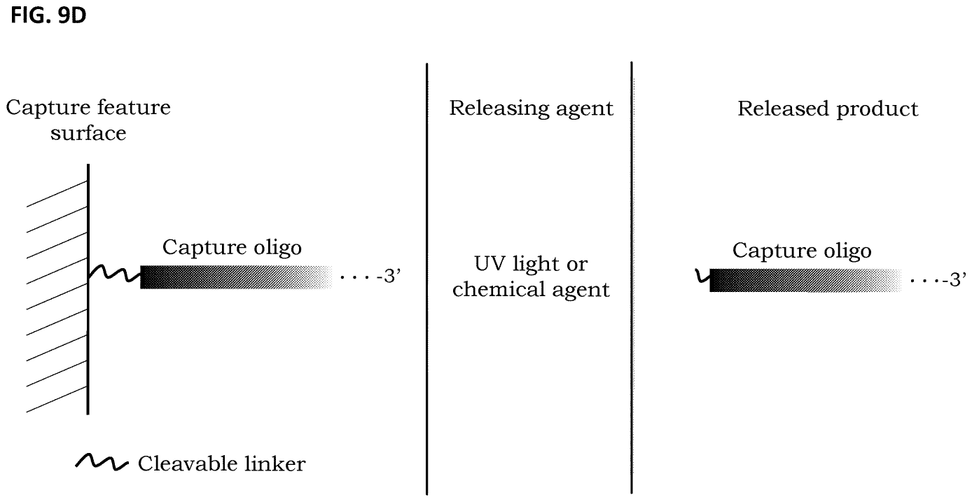

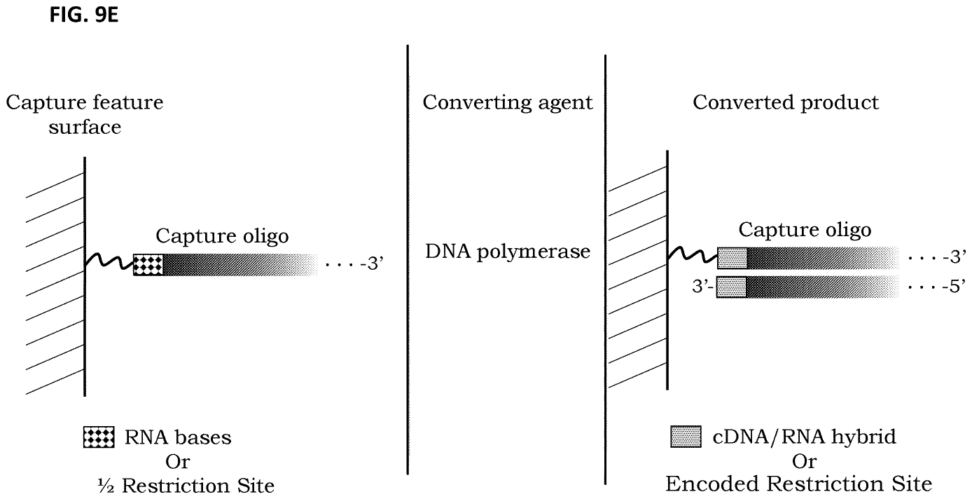

The release of spatially-encoded products from the spatial capture array (FIGS. 9A-9E show a variety of ways to do this), which can be performed within the same isothermal reaction mixture. This can be accomplished through the inclusion of RNA bases within the 5′ region of the capture oligo, such that extension by the polymerase through this region creates a new DNA/RNA hybrid species, whereby the new DNA/RNA hybrid species becomes a substrate for RNase H already included in the one-pot isothermal information transfer reaction (FIGS. 9A and 9E). This can also be accomplished through the inclusion of an encoded restriction enzyme site (at the 5′ end of the single stranded capture oligo), such that extension by the polymerase generates a dsDNA restriction site for recognition and cleavage by a corresponding restriction enzyme during the one-pot isothermal information transfer reaction, or after the extension step has been performed (FIGS. 9B and 9E). The inclusion of a photo- or chemically-cleavable linker for attachment of 5′-end of the capture oligo to the capture feature enables a third mechanism for release of spatially encoded products (FIGS. 9C and 9D).

A further benefit is that no user handling is required for product retrieval. In previously known workflows, following capture the two surfaces are separated, the capture array is washed, and captured probes are eluted from the capture array with elevated temperature. The methods disclosed here obviate the need for any of these steps.

Not all of the capture oligos may be modified to contain 5′-RNA bases for spontaneous release of products into solution following spatial encoding, but rather, perhaps a certain % of capture oligos at each capture feature (on each bead here) contain 5′-RNA bases for spontaneous product release, while a certain % of these contain all DNA bases, but are attached by a photo-cleavable linker to the capture feature. This embodiment describes is a very useful capability. This enables a user to come back later to an interesting sample that was screened with a relatively “shallow” read depth (i.e., not all of the captured information was released during the isothermal reaction, but the resulting library was sequenced and a digital image was reconstructed from this partial read), and to select a region of interest (ROI) through the software displaying the digital image. It is then possible to employ an instrument for defined, site-specific photo-release of all remaining spatially-encoded products within the selected region of interest. This has been accomplished, for instance, by using digital micromirror arrays for focusing UV light wavelengths at defined locations across a surface, and this type of array can be fabricated or purchased commercially with 10 μm resolution or higher. Additional data may then be obtained from the remaining sample that is thusly released.

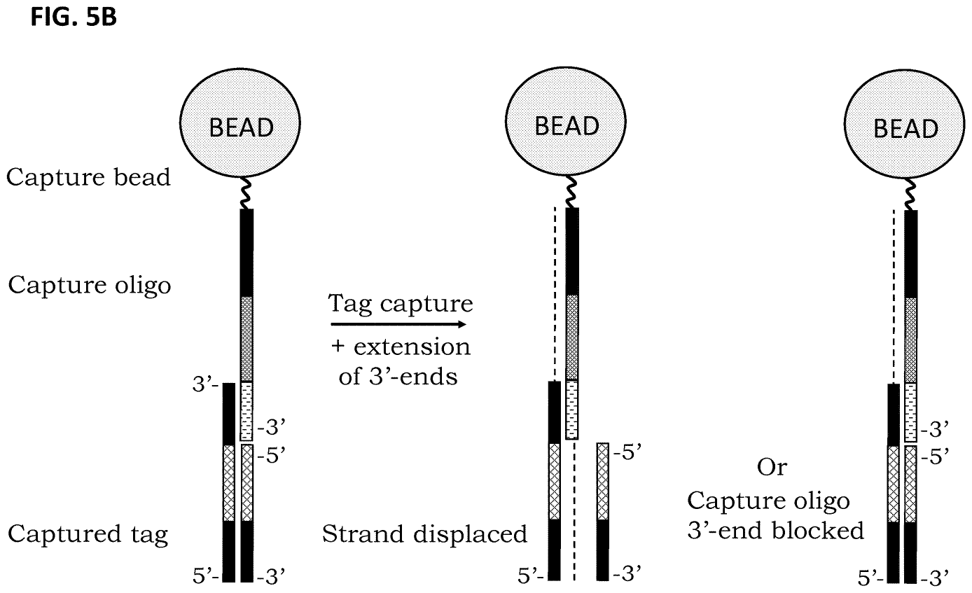

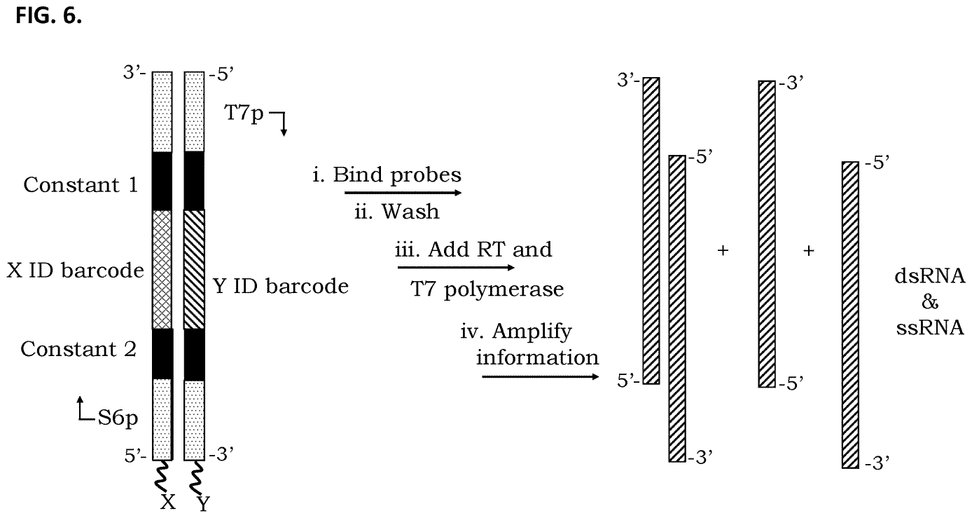

Also contemplated as a way to expand the application of the technology(s) described herein is to provide compatibility between other DNA-encoded molecules (those that do not contain the tag features described herein), and the isothermal spatial encoding system described here. To do this, molecule ID tags may be designed to include RNA bases within them (FIG. 5A), which will work seamlessly with the one-pot isothermal information transfer reaction described here (FIG. 5B). In one example, this is achieved through the inclusion of a stretch of 5′-RNA bases within a linear (non-hairpin) tag attached to the encoded molecule library. This tag type, going from the 5′-end to the 3′-end of the tag, contains a 5′-linker used for attachment of the tag to the molecule library, which is followed by a stretch of 5′-RNA bases that encode the reverse complement of the sequence to be captured (a constant region on all tags), followed by a variable region that encodes the individual molecule attached to the tag, followed by a second constant region made of DNA bases. Before or following a binding assay (screen or selection) a dsDNA region can be generated by hybridization of a complementary oligo to the 3′-constant region of the tags, or installed as a constant hairpin structure. This constant “primer” or hairpin structure at the 3′-end is best installed prior to the binding assay, and can be extended via an extension reaction by a polymerase (containing both RNA- and DNA-dependent DNA polymerase in order to extend through both base types contained within the tags) to generate a DNA/RNA hybrid region at the 5′-end of the tags, proximal to the attached molecule library. The extension reaction can be performed before or after the binding assay. Following binding, the tagged probes or molecule library can be spatially-resolved using the approach described above. Upon introduction of an enzyme containing RNase H activity, these tags will be liberated from the sample surface in the same way as described above, which reveals the now 3′-end of the extension product (constant capturable region on all tags), and are therefore made competent for inclusion within the same isothermal information transfer workflow described here. Other sequences can also be designed for inclusion within tag sequences, as shown in FIG. 6, which have many additional benefits outlined below. In FIG. 6, two opposing RNA polymerase promotors are used, which will be most optimal.

Additional ways to accomplish this involve adding an adapter molecule to other DNA tags in order to make them compatible with the one-pot isothermal information transfer reaction. These various adapter molecules may come in different forms, examples of which are described:

The first form of adapter is simply the herein RNA/DNA hybrid hairpin tag appended to the free end of the DNA that is encoding the probe (FIG. 1). This adapter type can be fused as a contiguous polymer with other DNA tags, or can be attached through a compatible covalent reaction chemistry to other DNA tags or molecules. In this case, the tag barcode would now be encoded within the loop of the hairpin attached to the DNA-encoded probe. To convert and encode antibodies or other smaller panels of probes (no larger than tens of thousands of probes in a mixture), including reagents from other companies (such as SomaLogic), or specific transcriptomic probes, this will work. This form of adapter has already been shown to work well within the one-pot isothermal information transfer reaction.

### Amplification:

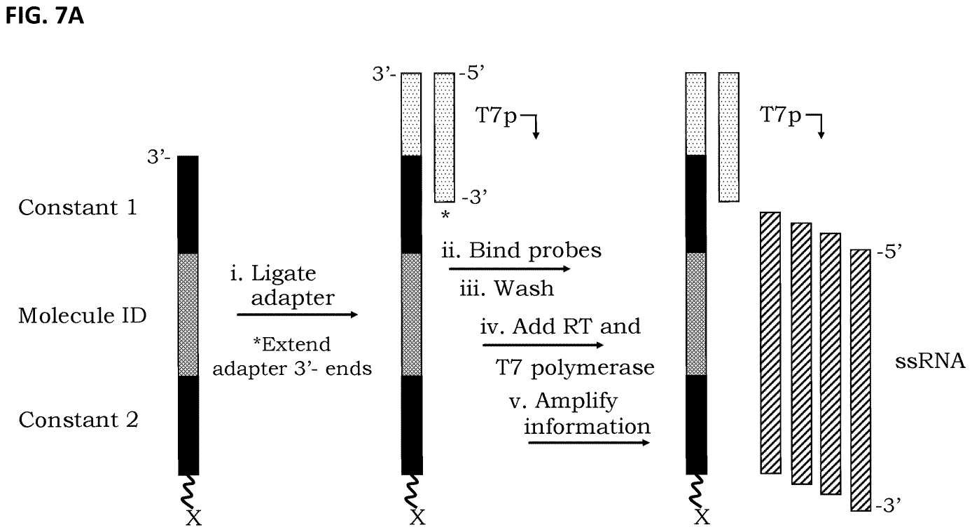

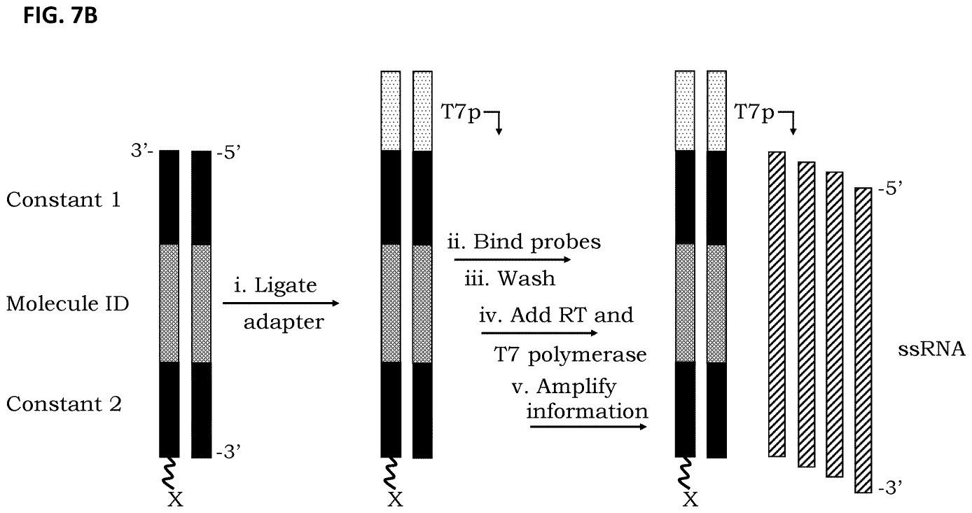

The second form of adapter imparts an amplification feature within the method. Here, an adapter containing a polymerase promoter sequence (such as T7 or S6 for example) is ligated onto the DNA strand of the DNA-encoded molecules thorough a templated ligation reaction (FIGS. 7A-7B). To accomplish adapter ligation to ssDNA tags (FIG. 7A), the sequence information for ligation must be known, as the ligation reaction must be templated by a splinting sequence. Fortunately, most encoded molecule libraries contain two constant regions within their DNA tag structures (for their downstream retrieval following a screen or selection by PCR amplification) and these constant regions flank the information corresponding to the encoded library member. One of these constant regions (region 1) can be used for ligation of the adapter, and the other constant sequence (region 2) can be used for capture. For ligation of this adapter type to dsDNA tags (FIG. 7B), the sequence information of the constant regions isn't necessary. Once ligated, this promotor sequence is competent for initiation of transcription of the probe-encoding sequence upon introduction of the appropriate polymerase. For the T7 adapter example, this adapter may consist of a double-stranded DNA sequence encoding the T7 polymerase promoter, with an overhanging compatible “sticky end” for ligation to the encoding DNA strands of the molecule library. Alternatively, this adapter may consist of a single contiguous DNA strand within a hairpin structure, whereby the stem of the hairpin (dsDNA region) encodes the T7 polymerase promoter sequence, and whereby an overhanging compatible “sticky end” for ligation to the encoding DNA strands of the molecule library is available for attachment. The same structures can be used for encoding any polymerase promoter sequence. This type of adapter may be ligated onto encoding DNA strands of the molecule library prior to or following the binding of the probes to a sample surface. After washing unbound probes from the sample, the same type of capture surface used in previous examples can be placed in proximity with the probed sample.

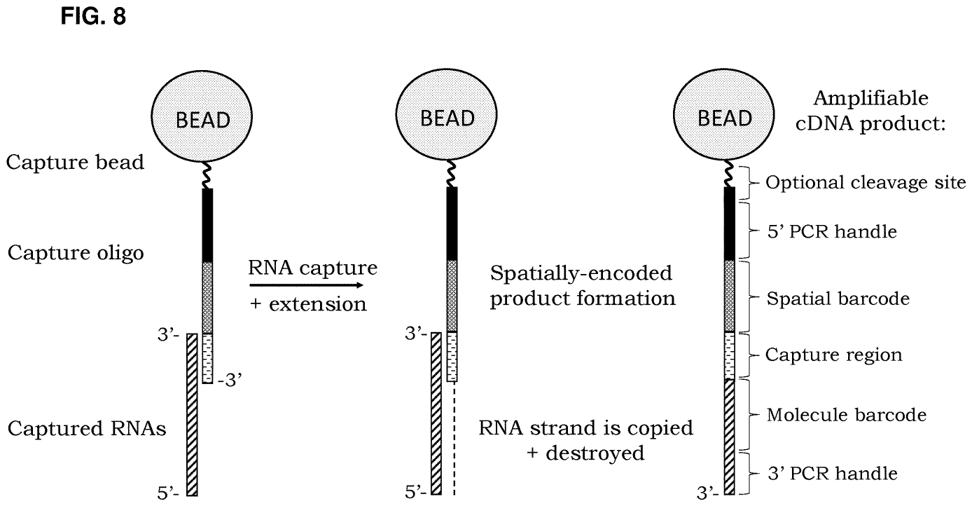

To initiate this isothermal information transfer reaction, the appropriate polymerase (T7 RNA polymerase here) is introduced with appropriate co-factors (NTPs for transcription in this case, Mg²⁺, etc.), and up to 1,000 copies of RNA are estimated to be produced from each adapter-modified DNA tag bound to the surface (through its associated probe). In this case, 3′-capture regions of capture oligos within the capture array are designed to specifically capture RNA produced from the encoding tags via the second constant region that was not used for ligation of the adapter (FIG. 8). RNA produced by this reaction is captured proximally onto spatially encoded capture features in the presence of a polymerase containing reverse transcriptase activity. Upon contact (capture of RNA onto capture oligos), the reverse transcriptase enzyme extends information contained within the released RNA onto the capture oligos of the capture array, by extension of capture oligo (CO) 3′ ends. This process generates a contiguous DNA molecule containing the (amplified) information released from the adapter-modified probe (probe or molecule ID barcode), as well as information from the spatially-encoded CO molecule that was most proximal to that location of the sample surface. The constant region of the encoding tags used for ligation then becomes one of two PCR handles for library amplification, while the other PCR handle is provided on the capture oligo. These contiguous molecules make up the spatially encoded library, and can be released by chemical cleavage, by photolysis, RNase H, or restriction enzyme cleavage from the capture array into solution (FIGS. 9D and 9E). The spatially encoded library products are then retrieved by aspiration of the solution as described above, for downstream NGS library preparation and sequencing.

### Application of the Method to Include Transcriptomics

This general first approach (the DNA/RNA hybrid hairpin tag approach) can be expanded to include probing of transcriptomics within the described spatial-encoding workflow. This approach consists of using probe pairs that recognize immediately adjacent RNA sequences of a target RNA (this has been done by others). Each probe pair, once recognizing their target RNA sequences, are designed to create the described type of hairpin structure only when bound to their target in proximity; the probe-bound RNA creates a template for proximal ligation of matching probe pairs to generate the disclosed hairpin structure. When multiplexing, each hairpin formed by proximity-initiated ligation of probe pairs contains a barcode in the loop region that corresponds to the RNA being probed. To accommodate larger libraries of tens of thousands of probes, the barcode in the hairpin loop may be expanded to 8-10 bp. The isothermal information transfer reaction is initiated in the same manner as with antibody probes tagged with the DNA/RNA hybrid hairpin oligo tags. This may ultimately enable a multi-omic assay format that can be performed on the same sample surface simultaneously, using the described isothermal information transfer reaction. If not simultaneously, for some sample types this approach can be performed in tandem (first probe RNA, then protein from the same sample, or vice versa) or on serial sections for tissue assays, if necessary or desirable.

Other versions of generating RNA/DNA hybrid species by proximity are also designed and shown. These tag types may also be useful for probing nucleic acid sequences in samples, and/or for determining interactions between tagged molecules with spatial resolution across a sample, when used within a similar workflow. These can consist of two complementary regions, which are forced together due to proximity, designed such that they do not generate complementarity at the temperature of the hybridization step (these complementary regions are less than 10 bp in length, whereby one of the two regions of complementarity consists of RNA bases and the other consists of DNA bases within the region of complementarity). Therefore, when used in this workflow, their proximity is generated through probe specificity for each probe's respective target, and if two tags are brought closely together (close enough to generate the RNA/DNA species by proximity), information regarding their proximity can be released upon contact with an enzyme containing RNase H activity, and these can be spatially encoded through capture of the released information using the downstream steps of the isothermal spatial encoding workflow described above.

### Spatial Encoding Surface

As described here, a spatially-encoded capture surface in some instances is an array of features, each including bound capture oligonucleotides. The features of the array may include randomly arrayed beads packed at high density, or a spotted microarray, or may utilize both technologies for total surface expansion (described below). The capture oligos (CO) at each feature of the capture array contain a capture region (CR) for capture of released information from the sample by base complementarity. This CR may be common to all COs contained within the spatially-encoded capture surface. Every CO also contains a spatial barcode (SB) that is unique to each individual feature (common to every CO within the lawn of COs at each feature location), which is used for ascribing capture feature locations by one or more forms of sequencing. COs also contain one of two constant regions (CR) common to every CO in the array that can be used as a PCR handle (PH), which is required for retrieval and/or amplification of spatially-encoded products following the spatial encoding reaction. COs may also contain a stretch of contiguous or non-contiguous randomized bases that include a unique molecular identifier (UMI), which can be used for elimination of PCR-generated amplification bias (well known in the art; see, for instance, Kivioja et al., Nat. Methods 9:72-74, 2012, doi.org/10.1038/nmeth.1778). One preferred structural orientation of nucleic acid elements of COs within the capture array going from 5′- to 3′- is: a 5′-linker for attachment of the CO to the surface (feature), followed by an optional stretch of 4-8 5′-RNA bases, followed by the one of two PH regions, followed by the SB region, followed by the CR used for capture of released information from the sample. In this way, released information from the sample during the isothermal spatial encoding reaction by RNase H or by RNA amplification can be captured onto COs at each spatially-resolved capture feature.

Though embodiments are provided herein that use eight (8) RNA bases, it is contemplated that this number may be reduced, possibly down to the length at which RNAse H can cleave (which in instances is as short as a single RNA base from a hybrid strand). Also contemplated are embodiments in which the number of RNA bases is increased beyond 8, to expose more (capturable) bases following extension and release of spatially-encoded extension products (as described herein).

In the case where RNase H is used, the 3′-ends of oligos captured from the sample can be extended by the polymerase to generate products consisting of contiguous sequences containing both the information of the released probe, as well as the locational information provided by the capture feature. Additionally, both of the two PHs required for downstream retrieval and/or amplification of the spatially encoded products.

In the case where tag information amplification by a first polymerase is enabled through the installation of an adapter region onto probe nucleic acid tags to contain a competent promotor region (as described above in 5 under Amplification this is T7 RNA polymerase), released information from the sample is captured through a newly synthesized sequence that is common to all probe tags. COs in the capture array contain a common 3′ CR for capture of released information from the sample through complementary base pairing. In this case, an enzyme containing RNA-dependent DNA polymerase activity (reverse transcriptase activity) is included within the same reaction mixture, and is used for extension of CO 3′ ends to contain the information templated by the RNA products released from bound probes. These fully extended COs that have encountered a released RNA sequence cannot be extended again. Additionally, RNase H activity within the reverse transcriptase enzyme will destroy released RNAs following their copying via the extension reaction, and therefore should by destroyed which will decrease the chance that they will act as a template for a subsequent extension reaction. This effectively results in one sequencing count per released RNA.

In the case whereby a stretch of 4-8 RNA bases is included within the 5′-end of the COs of the capture array, a polymerase enzyme containing both RNA- and DNA-dependent DNA polymerase activity (or two polymerase enzymes that together contain these activities) is required for extension of captured oligos through CO regions that consist of both base types in the CO template. Preferably, one enzyme that contains both activities (such as Maxima™ RT polymerase) is used under conditions that are compatible with other enzymes used in the isothermal reaction mixture.

Optionally some percentage or all of the COs contain this stretch of 5′-RNA bases for spontaneous release of extended, spatially-encoded products into solution by an enzyme containing RNAse H activity.

Optionally some percentage or all of the COs are attached to features of the capture array via a photo-cleavable linker, and these extended and spatially-encoded products may be released after the isothermal spatial encoding reaction has completed.

Optionally some % or all of the COs contain a 5′ restriction enzyme (RE) site that is formed following extension by a polymerase enzyme (which does not necessarily need to possess any RNA-dependent DNA polymerase activity, just DNA-dependent DNA polymerase activity), whereby spatially-encoded products may be released by a RE that recognizes this newly formed site following extension by the polymerase. This RE may be included within the same reaction mixture as the isothermal information transfer reaction, or introduced after the isothermal spatial encoding reaction has completed.

Different versions of direct visual sequencing of spatial barcodes contained within features of the capture array that can be used for determining locations of randomly-arrayed capture features may include those known in the art (sequencing by synthesis (SBS), sequencing by binding (SBB), or orthogonal cleavage sequencing (OCS) as described in WO 2022/187719, for example). This enables identification of each individual feature location within the capture array, and these locational coordinates are recorded as unique contiguous DNA sequences within a software package that generates a physical map of the feature locations (not unlike a microarray). This product can be used as a capture surface, but preferably, subsequent modifications of the capture array are performed. Determining locations of randomly-arrayed capture features (such as beads coated with capture oligos that are randomly arrayed on a surface) is not required for microarrays because locations of capture features are pre-determined.

For direct sequencing using SBS or SBB methods, locational barcodes within capture oligos can be include 30 or fewer contiguous bases. That is because the locational barcode within the capture oligo may be directly sequenced by the visual decoding method (next generation sequencing). However, visual barcodes built for use in decoding by OCS experiments are coordinately constructed along with capture oligo spatial barcodes across three rounds of splitting and pooling of bead-based libraries. This approach is first described in WO 2022/187719; its use toward encoding capture oligos for next generation sequencing applications is further described here (see Appendix A included in priority U.S. Provisional Application No. 63/487,575, filed on Feb. 28, 2023). In brief, barcode segments consisting of dsDNA containing designed cleavage sites (for RE recognition) are attached to one of 5 detectable labels (identimers) and are arranged in a plate or in tubes, whereby the 5 differentially-labeled identimer segments are mixed 1:1 in combinations without redundancy. For example, AF405-labeled Id1 is mixed with AF488-labeled Id1 or AF550-labeled Id1 or AF647-labeled Id1 or AF750-labeled Id1, but not with more AF405-labeled Id1. This will improve upon their visual detection during OCS experiments. This will be done for each differentially-labeled identimer segment in the chain to provide a total of 10 equally mixed dual-colored options at each segment. Here, 6 identimer segments of this type will be used for generating a combinatorial library of 1M beads. This bead library will be generated over 3 rounds of splitting and pooling instead of 6 (for 6 identimer segments) because segments Id1 and Id2 will be pre-ligated to form 100 options (Id1+2) for identimer ligation round 1, segments Id3 and Id4 will be pre-ligated to form 100 options (Id3+4) for identimer ligation round 2, and segments Id5 and Id6 will be pre-ligated to form 100 options (Id5+6) for identimer ligation round 3, which will generate a total library diversity of 1M differentially labeled beads after only 3 rounds of splitting and pooling. Here, each cycle will remove two different labels from each bead, enabling robust results from OCS experiments. Before, during, or after attachment of the first round of identimers to beads, 100 different single stranded oligo nucleotides are attached to beads (using either the same or using unique/orthogonal/non-competing attachment chemistries) to distinguish each of the 100 different options (Id1+2) by a unique nucleic acid sequence. This single stranded nucleic acid sequence represents the first segment of the NGS capture oligo that will be constructed in coordination with the visual identimer barcode. The first segment of the NGS capture oligo (NGS1) contains a 5′ attachment chemistry compatible for attachment to the beads, an optional photo-cleavable linker, followed by optional inclusion of RNA bases within the 5′-end of the oligo (constant region made of either RNA or DNA), followed by one of two PCR handles required for downstream PCR (constant region), followed by a 5-base sequence that is used for encoding each of the 100 identimer combinations (ld1+2), followed by a 4 base site common to all 100 different versions of NGS1, which is designed for orthogonal ligation to a subsequent NGS barcode segment. After attachment of the first coordinated (co-encoded) identimer segment (ld1+2) with its corresponding NGS segment (NGS1), beads will be washed, pooled to mix and split into the next set of 100 different options. Due to the fact that enzymatic ligation of subsequent identimer and NGS barcode segments can be made entirely orthogonal, these reactions can take place simultaneously within the same reaction mixture. For example, identimer segments contain sticky ends for ligation that are of a different length and base composition than sticky ends that are generated for NGS segment ligation. This is done not only by carefully designing sticky ends for each ligation (identimers versus NGS segments) to be orthogonal by base composition and by length, but also by templating NGS segment ligation using RNA splints. This allows for T4 DNA ligase (whose preferred substrate is the dsDNA of the identimer segments) to be used along with the SplintR enzyme (whose preferred substrate is the RNA-splinted DNA-DNA ligation junction). This should further improve upon specificity of reactions taking place for both segments when carried out in tandem, or at the same time within the same mixture. Therefore, orthogonal enzymatic ligation of Id3+4 and NGS2 can be performed at the same time in the same wells, whereby Id3+4 consist of 100 different options encoded within the spatial barcode region of NGS2. NGS2 contains a 5′ region with complementarity to a constant RNA splint that will be used for templated ligation of NGS2 with NGS1 on the bead, as well as a region encoding each of the 100 different options corresponding to Id3+4, followed by a 3′ end for templated ligation to the next NGS segment in the chain. Beads will then be washed, pooled to mix, and split into the final 100 different wells for orthogonal ligation of Id5+6 and NGS3 segments. NGS3 contains a 5′ region with complementarity to a constant RNA splint that will be used for templated ligation of NGS3 with NGS2 on the bead, as well as a region of 5 bases encoding each of the 100 different options corresponding to Id5+6, followed by a 3′ end for capture, or for templated ligation to the next NGS segment in the chain (outlined under Capture array below). Beads will then be washed and pooled to form a library of beads with a diversity of 1M, whereby each bead type in the library contains a unique sequence of visual labels with a coordinated NGS capture oligo containing DNA sequence information corresponding to the color code (identimer chain).

Benefits include: significantly reduced time and cost for reading beads via OCS workflows when compared to SBB or SBS (next generation sequencing). This is because the OCS workflow required for reading identimer chains sequences on beads involves introduction of different unique/orthogonal/non-competing restriction endonucleases (REs) at each cycle followed by imaging. These REs are significantly less expensive than components required for next generation sequencing. Additionally, all cleavage agents required for reading beads via the OCS workflow required here can function in the same reaction buffer (1× CutSmart® buffer from NEB). Furthermore, the entire OCS bead decoding workflow occurs at a low isothermal reaction temperature (37° C.), and therefore does not require elevated temperature or any changes to temperature during decoding. Finally, the cycle time for decoding identimer chains of this type is around 5 minutes per cycle; to decode identimer chains of 6 segments as described here, via the required OCS workflow will take about 30 minutes. Following the decoding of identimer chains on beads to determine bead locations on a surface, the NGS capture oligo containing the information regarding which identimer chain was on that bead (the color code) remains intact for subsequent capture of nucleic acids (during the isothermal spatial encoding workflow described here for example).

Alternatively, bead locations can be determined using traditional NGS reagents as mentioned above. For traditional NGS sequencing experiments, expensive reagents must be used for accurate determination of DNA sequences, which include reversibly-terminated and/or labeled nucleotides, elevated temperature is required during decoding (usually 65° C.), multiple different reagents are introduced during each decoding cycle and these reagents cannot be mixed (cleavage solution cannot be mixed with incorporation solution for example) requiring more sophisticated fluidics, and NGS cycle times take significantly longer than 5 minutes each. To read a large surface area of 1 μm beads via NGS, for example a 1.75 cm×1.75 cm surface of packed 1 μm beads where there will be about 10B individual 1 μm beads, the length of the NGS locational barcode must be over 24 bp to reach a diversity of at least 200T different beads in the library (potential locational barcode combinations) to reduce the chances of barcode redundancy within the large surface area, which ends up requiring many more than 6 decoding cycles.

In exemplary existing technologies, all beads are labeled with different combinations of fluorophores prior to decoding by OCS (e.g., as described in WO2022/187719), so high dynamic range (HDR) imaging should be used when determining identimer chain sequences via OCS workflows for accurate decoding. With the described identimer chains and OCS workflow, the bead library diversity is only 1M, so how with such a low diversity of beads can a 1.75 cm×1.75 cm surface area be covered with the capture array? This is solved using a semi-patterned bead array, whereby x and y coordinate oligonucleotide barcodes are printed directly onto randomly arrayed beads within each feature of the semi-patterned bead array. These x and y coordinate barcodes are ligated directly onto bead capture oligos, and impart additional locational information to beads within each feature of the semi-patterned bead array. This is more clearly outlined in Capture array below. A problem with this approach is that each randomly arrayed bead capture array must be read individually, and this could be made much more amenable to manufacturing of many arrays by copying of the information contained within the bead capture oligo arrays onto an acceptor array. This is described in more detail below.

### Capture Array:

The coordinated identimer/NGS bead library can be immobilized on a surface at a desired density, using one of many different attachment chemistries (covalent, non-covalent), for downstream visual encoding and decoding experiments, such as methods using OCS. Preferably, beads are randomly arranged within a semi-patterned fashion, whereby patterned features of the array consist of defined regions for bead immobilization. These bead immobilization features may consist of squares or other shapes of defined size (for example 100 μm×100 μm squares, whereby ~1,000 packed 3 μm beads may be randomly arrayed, or 35 μm×35 μm squares whereby ~1,200 1 μm beads may be randomly arrayed). Once arrayed randomly within the squares, a droplet printer may add additional oligos to the array in x and y coordinates. These x and y coordinate oligos are to be ligated directly onto NGS barcodes on the beads to impart additional locational information. For example, an array containing 35 μm×35 μm squares spaced by 1-3 μm, arranged in a grid of 500 squares×500 squares, will cover greater than 1.75 cm×1.75 cm in total surface area (FIG. 10). Each square of the grid will receive a unique combination of oligos, whereby the x coordinate oligo is ligated through a splint to the NGS barcode of the bead which contains the identimer chain information. The x coordinate oligo is added to rows of the grid first, and contains a 5′ region used for templated splint-ligation to the NGS₃ barcode (3′ acceptor region) of the bead, as well as a 5 base region corresponding to the row of the grid, and a 3′ region for templated splint-ligation to the y coordinate oligo. The y coordinate oligo is added to the grid after the x coordinate oligo has ligated to the beads, and contains a 5′ region for splint ligation to the x coordinate oligo on the beads, a 5 base region corresponding to the column of the grid, and a 3′ capture region for capture of oligos (during the isothermal information transfer reaction described here for example). Therefore, through the use of additional spatial information imparted by the x and y coordinate oligos deposited into the squares, very large surface areas can be covered without risking bead redundancy, and by reading relatively low diversity libraries of differentially barcoded beads (1M described here). The grid of squares can be expanded to increase the total surface area of the capture array. This overall approach (the grid of squares containing randomly arrayed beads) reduces the overall diversity of beads required to produce a capture array of large surface area, and significantly reduces the time required to read each capture array.

Reading locations of the identimer/NGS coordinately-encoded beads: Identimer chains on beads can be distinguished by imaging before and after exposure to one unique/orthogonal/non-competing RE at a time in a series of cycles (described in WO2022/187719 and briefly herein). Importantly, the cleavage agents used (such as REs) will not cleave the NGS barcodes. Therefore, following OCS experiments used for reading bead locations, the corresponding NGS capture oligos encoding the locations of the beads will remain intact and competent for capture of information released during the one pot isothermal information transfer/spatial encoding reaction.

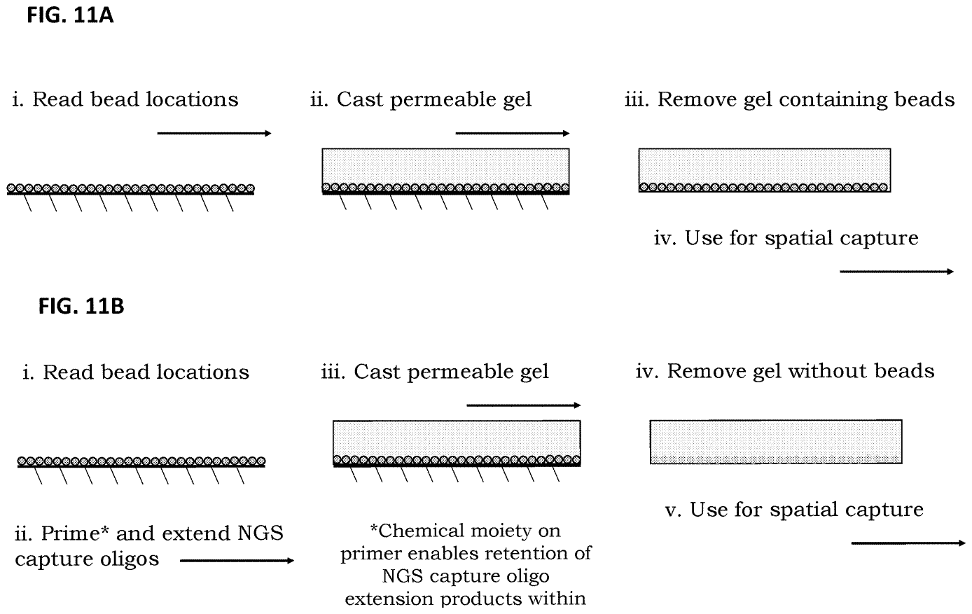

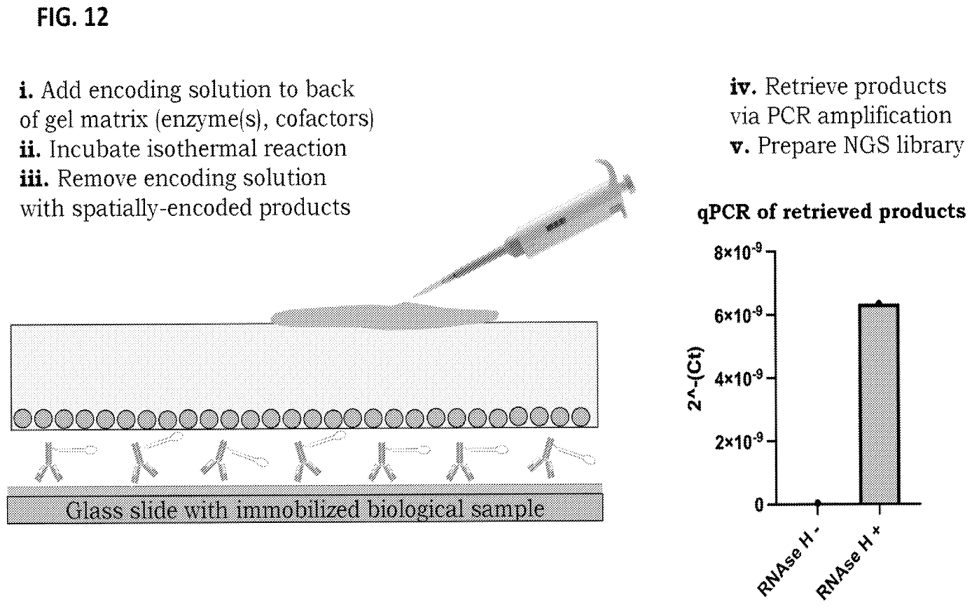

Use of the capture array: there are a variety of ways the spatially-encoded capture array can be made functional for use. Following the reading of bead locations within the semi-patterned random bead array, beads may be embedded in a gel, peeled off of the surface from which their locations were determined (as in examples shown here), and used as a capture array directly (FIG. 11A). Rigid surfaces such as a patterned silicon wafer, glass or plastic are less suitable for the information transfer reaction. This is because these rigid surfaces will not conform well to the sample as these surfaces are not pliable (like a gel or membrane). Additionally, it is difficult to maintain bead immobilization on rigid surfaces during the transfer reaction, even when beads are covalently bound to the capture array surface. That is because in order to achieve sub-cellular resolution with any information transfer approach, the capture array must be very close to (within just a few microns or touching) the sample. This physical contact can literally wipe immobilized beads off of a rigid surface. However, when beads are embedded within a pliable matrix such as a gel, then peeled off of the surface from which they were originally immobilized, the beads tend to stay within their original relative locations during the transfer reaction. Furthermore, due to the isothermal nature and potential completeness (in one version, the spatially encoded products are released from capture beads during the isothermal reaction), a semi-permeable gel can be used throughout the reaction. In this way, reagents (enzymes and/or their necessary cofactors) required for initiating and/or carrying out the complete isothermal information transfer reaction can be introduced across the semi-permeable gel matrix by diffusion. By simply incubating the array and sample (sandwich) in the isothermal information transfer reaction solution at isothermal temperature (37-42° C.), the reaction proceeds and products can be released from the capture features into the surrounding solution, enabling their diffusion through the gel and their subsequent retrieval by aspiration of the solution (FIG. 12). See also Appendix A included in priority U.S. Provisional Application No. 63/487,575, filed on Feb. 28, 2023.

After aspiration, additional analysis may be as simple as adding isothermal reaction components directly to beads containing attached RNA/DNA hybrid hairpin tags, or to samples with bound antibody-tag conjugates, incubating at the isothermal reaction temperature for a time period, then pulling that solution off of the sample surface (or beads via magnet), and capturing the released tags in the aspirated solution on capture beads containing capture oligos that are added to the aspirated solution. These tags are captured in the presence of all isothermal reaction components at the temperature of the reaction, so the tags are extended on the capture beads to contain the spatial barcode region and PCR handle encoded within the capture oligo. Those full-length extension products are then cleaved from the beads via RNAse H activity (capture oligos contain 5′-RNA bases to enable this following the extension reaction) and these products are competent for amplification using the designed primers.

There are also ways to improve the stability of the beads (how well they remain bound) within the gel matrix beyond what is shown here. Exemplary approaches are described below.

In examples shown here, the spatial encoding surface consists of 1 μm beads containing a single capture oligo type (without spatially encoding), either bound to a glass surface or embedded within a gel (0.5-2% agarose/10 mM Tris pH 7.5). These examples prove the biochemistry of the “one-pot” isothermal reaction. To make robust spatially encoded capture arrays, their production can be industrialized and required software components developed for reading bead locations, and for reconstructing digital images using next generation sequencing counts.

Constructing a Capture Oligo by Printing x- and y-Coordinate Oligos

As described elsewhere (here, but also in the OCS PCT application), visual barcodes can be operationally-coupled to capture oligos on the same beads, whereby the visual code is encoded within the DNA sequence of the corresponding capture oligo. To expand the surface area of a capture array containing monodisperse, randomly-arrayed capture beads of low diversity (below 10M different beads for example), it is possible to create sub-arrays of the beads for further spatial addressing (illustrate herein in FIGS. 17A-17C). The creation of sub-arrays of defined size can be accomplished using photolithographic techniques that are known in the art, using substrates ranging from silicon wafers, to glass, and alternative methods can be used to manufacture subarrays from PDMS. Further spatial addressing can be achieved by droplet printing of x- and y-coordinate oligos into known locations, whereby unique combinations of x- and y-coordinate oligos correspond to specific sub-arrays. In this example, the visual barcode (VBC) region of the capture oligo would contain (from 5′ to 3′) an attachment moiety and linker, a stretch of RNA bases, a 5′ PCR handle, a variable region (stretch of DNA bases) specific to the visual barcode on the bead, followed by a region (stretch of DNA bases) with complementarity to the x-splinting oligo used for ligating the x-coordinate barcode (x-BC) oligo. After droplet printing a solution containing a ligase enzyme (preferably the SplintR enzyme) and a unique x-BC oligo across the sub-arrays in one direction, this oligo would be splint-ligated to the VBC oligo via base complementarity between the splinting oligo (preferably an RNA splinting oligo) and the 3′-end of the VBC oligo. The x-BC oligo would contain (from 5′ to 3′) a stretch of DNA bases with complementarity to the x-splinting oligo, a unique x-coordinate oligo barcode region (differs between each x-BC oligo), and a stretch of DNA bases with complementarity to the y-splinting oligo. Each row (if printing in the horizontal direction) or column (if printing in the vertical direction) of sub-arrays would receive a unique x-BC oligo for attachment to the VBC oligos on the beads by splint-ligation. Following ligation, the array (containing all sub-arrays) would then be washed and prepared for printing of a unique y-coordinate barcode (y-BC) oligo into each of the sub-arrays. The y-BC oligos would then be printed, for instance, in the same manner as the x-BC oligos (with a ligase enzyme in a buffered solution), but perpendicularly to the orientation in which the x-BC oligos were printed. This would result in a unique combination of x-BC and y-BC oligos in every sub-array. The y-BC oligo would contain (from 5′ to 3′) a stretch of DNA bases with complementarity to the y-splinting oligo, a unique y-coordinate oligo barcode region (differs between each y-BC oligo), and a stretch of DNA bases with complementarity to released hairpin tags (capture region). Following ligation, the array (containing all sub-arrays) would be washed and prepared for optional splint removal. If splinting oligos are composed of RNA, and the ligation step is carried out using the SplintR enzyme, remaining splint oligos can be removed with RNAse enzymes, or a denaturing step to strip the splints off of capture oligos. Visual barcodes can be decoded before or after x- and y-coordinate oligos are ligated to impart additional spatial information to visually-barcoded beads.

### Attenuation of “High” Abundance Signals

Also contemplated herein are Attenuation Tag-like nucleic acid molecules and their use to attenuate signal(s) that arise from high-abundance targets in an analysis. In general, such Attenuation Tag-like nucleic acid molecule differs from a Hairpin Tag nucleic acid molecule or DNA/RNA Hybrid Hairpin Tag nucleic acid molecule by lack of a functional cleavable site—that is, replacement of the RNA base(s) that enable cleavage/release by RNAseH or omission/modification of the restriction endonuclease recognition site that serves as the cleavable site of the tag.

When multiplexing probes or conjugates for spatially encoded read-outs, this type of attenuation is used to “see” (detect, read out) all tag sequences, whether they are in high abundance or low abundance. This type of probe attenuation has been demonstrated previously, but never in a system such as described herein.

For example, the assays herein can be used in an attenuation format by including “all DNA base” tags mixed in (for instance, in a pre-determined portion or precent) with Hairpin DNA/RNA Hybrid Tags, such that these conjugates behave the same in the assay (having similar size and structure, including any hairpin structure), but the signal from targets of high abundance can be attenuated. Similarly, signals from Hairpin Tags that rely on string of DNA bases comprising a restriction enzyme (RE) recognition site for cleavage can be attenuated by including similar tags, in which the RE recognition site is not included

Risks exist around expansion of probe content to large numbers of differentially encoded probes. As the described systems enable unprecedented expansion, it is important to account for highly expressed biomarkers, to not “drown-out” reads from biomarkers with low expression levels. For example, attenuation of probes with very high counts may be accomplished by mixing two conjugate types (one type will be conjugated with Hairpin Tags containing all-DNA bases and the other type will be conjugated with Hairpin Tags containing RNA/DNA hybrid regions) at a known ratio, which can be back-calculated later during analysis.

Improvements to bead stability (immobilization) within the gel matrix: beads can be made more stable within the gel matrix by adding components to the gel that specifically bind to moieties on the bead. For example, beads can be made multi-functional through chemical modifications to contain a variety of different unique/orthogonal/non-competing reactive chemistries which are solvent accessible. Tri-functional beads may contain a first reactive chemical handle for attachment to identimer chains, a second (unique/orthogonal/non-competing) reactive handle for attachment to NGS barcodes, and a third (orthogonal, that is unique to and different from the other two) reactive handle for covalent or non-covalent immobilization within the gel matrix.

Production of a capture array: Beneficially, new bead arrays would not need to be produced and read by OCS every time a capture array is made. A preferred method would involve generating the bead library as described above, immobilizing beads for reading their locations within the semi-patterned array as described above, but instead of embedding beads in the gel to physically transfer the beads within the gel (the previously described capture array above), the information contained within the NGS capture oligos is copied into the gel (FIG. 11B). This can be accomplished by creating NGS barcodes in the reverse complement orientation, where the capture region and PCR handles of the capture oligos are switched relative to each other. In this configuration, the capture region (in reverse complement) is oriented at the 5′ end, closest to the bead, and the constant PCR handle is oriented at the 3′ end, farthest away from the bead. A constant priming oligo with complementarity to the 3′ end of the reverse complement NGS barcode oligos can be hybridized to all beads on the surface. This priming oligo may contain a chemical modification for polymerization into a gel such as polyacrylamide. An extension reaction can then convert all of the reverse complement NGS barcode oligos into dsDNA. The full-length (correct complement) of the NGS barcodes would then be attached through hybridization to reverse complement NGS barcode oligos on beads. Acrydite-modified primers (this modification is available through commercial oligonucleotide vendors such as IDT) used for the extension reaction can be polymerized directly into polyacrylamide gels cast at various percentages. 4% polyacrylamide gels should allow for macromolecule permeability during the isothermal information transfer reaction. These gels can be cast very thin (<200 μm thickness), and NGS barcodes (locations and information) can thereby be polymerized directly into the gels. The cast gel can be peeled off of beads without removing the beads from the array surface (in the presence of a dsDNA denaturing agent such as >50% formamide, >3M urea, or 0.1M sodium hydroxide), which may enable gentle removal of gels from bead beds, which could enable many gels to be cast from the same bead array by repeating the extension and gel casting steps. Therefore, the most ideal capture array does not contain beads, but information from beads that was copied enzymatically and then subsequently transferred into a gel by polymerization (FIG. 11B). To impart rigidity to the 4% polyacrylamide gel or other gel which may ultimately be used, a semi-permeable membrane may be placed on top of the gel prior to polymerization or gel hardening, so as to create a layered “sticker” that can be used as a capture array. The rigidifying matrix may be composed of biologically inert material with defined pore size, such as cellulose, different meshes composed of suitable plastics, or PTFE for example.

Instrumentation. General instrumentation requirements for reading the herein described features (e.g., beads) are generally convention, including those described previously in WO 2022/187719, as well as descriptions of other DNA sequencing technologies. In embodiments, it may be beneficial that the instrumentation is able to scan a large surface area (up to 10 cm×10 cm due to the described grid system) packed with 1 μm beads, using high dynamic range imaging (HDR) to resolve immobilized barcodes, with the ability to see at least for instance up to 5 or more colors (such as standard fluorophores, in some embodiments) or many more in embodiments that employ quantum dots with appropriate filters during each decoding cycle. The cycling of solutions is performed under automated microfluidics, regulated by instrument control software that is also used for timing image acquisition.

Software. It will be recognized that the methods, systems, and workflows described herein are supported by, for instance: instrument control software (including for instance such as described in the literature, with modifications to accommodate the herein described coding and decoding cycles of features), software for image analysis used for decoding beads, software for generating 2D maps of bead arrays in the form of DNA sequences (the spatial barcode sequences) at each feature/bead location, software for aligning NGS reads to create lists based on spatial barcode sequences (for instance, to allow tag counts to be ascribes to physical locations in the 2D feature array maps), and software for displaying digital images through superimposing tag read counts onto physical locations in the 2D feature (bead) array map to generate digital images.

The term “orthogonal” refers to a component in a multicomponent system that has chemical reactivity with a particular reagent under a specific set of reaction conditions while at least one other component in the multicomponent system has limited or no reactivity with the reagent, even though all components in the multicomponent system are present in the same milieu. Additional terms used include “unique” (e.g., reactivities that act on different targets, such as REs that cut at distinguishable nucleic acid sites) and “non-competing” (e.g., reactivities that do not act in an overlapping or competing (for components, such as a reaction site) reaction, though they may occur under the same conditions). Isoschizomers are enzymes (usually, a pair) that are unique (as to the structure of the enzyme), but have the same target sequence recognition and cleavage activity as each other. Thus, two isoschizomers have the same biological activity but are (structurally) unique enzymes. Isoschizomers are not considered to be “non-competing”, since they cut at the same target sequence—and thus, they may not be appropriate for use in a single workflow as described herein.

Similarly, the phrase “orthogonal reactivity” refers to a component in a multicomponent system that has chemical reactivity with a particular reagent under a specific set of reaction conditions while at least one or more components in the system does not, even though all the components in the system are present in the same milieu.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments Set 1

1. A DNA/RNA hybrid hairpin tag having a structure as illustrated or described herein.

2. The DNA/RNA hybrid hairpin tag of embodiment 1, including (in 5′ to 3′ order) an attachment moiety, a string of RNA bases, a Tag PCR handle, a tag ID barcode, a Tag PCR handle complement sequence, and a string of DNA bases complementary to at least a portion of the string of RNA bases.

3. The DNA/RNA hybrid hairpin tag of embodiment 1 or embodiment 2, with a structure as illustrated in FIG. 1A, or as having the sequence of Tag v1, v2, v3, v5, or v6.

4. A released hairpin tag derived from the DNA/RNA hybrid hairpin tag of any one of embodiments 1-3, for instance as illustrated in FIG. 1B.

5. A tagged element, including an element to which is attached through the attachment moiety the DNA/RNA hybrid hairpin tag of embodiment 2 or 3.

6. The tagged component of embodiment 5, wherein the element includes one or more of: a biological molecule (such as a protein or a nucleic acid), a cell or tissue, an affinity molecule (such as an antibody), a bead, or another addressable feature.

7. A capture oligo (CO) having a structure as illustrated or described herein.

8. The CO of embodiment 7, including (in 5′ to 3′ order) an attachment moiety, a first string of DNA bases, a tag ID barcode, and a second string of DNA bases complementary to at least a portion of the first string of DNA bases.

9. The CO of embodiment 7 or embodiment 8, with a structure as illustrated in FIG. 2 or FIG. 3.

10. The CO of embodiment 8 or embodiment 9, which is attached to a capture feature through the attachment moiety.

11. The CO of any one of embodiments 7-10, further including a conditionally cleavable element.

12. A capture element, including an element to which is attached through the attachment moiety the CO of any one of embodiments 8-11.

13. The capture element of embodiment 12, wherein the element includes a bead or another addressable capture feature.

14. The capture element of embodiment 12 or embodiment 13, which is one capture element within an array of different capture elements, and wherein the CO on each of a plurality of the different capture elements of the array each include a different tag ID barcode.

15. A capture pair, including: a DNA/RNA hybrid hairpin tag having a structure as illustrated or described herein, or released hairpin tag derived from the DNA/RNA hybrid hairpin tag; and a capture oligo (CO) having a structure as illustrated or described herein,

wherein the sequence of the DNA/RNA hybrid hairpin tag and the sequence of the CO are at least partially complementary, such that when the hairpin tag is released in proximity to the CO, the released hairpin tag is captured by the CO.

16. A method for detection and/or quantification of targets in substantially two-dimensional (2D) sample as described or illustrated herein.

17. The method of embodiment 16, which is a “one-pot” method carried out essentially at a single temperature (that is, isothermally).

18. The method of embodiment 16 or 17, as illustrated (in whole or in part) in any of FIGS. 4A, 4B, 5A, 5B, 6, 7A, 7B, 8, 9A-9E, 12, 13, or Appendix A of priority U.S. Provisional Application No. 63/487,575, filed on Feb. 28, 2023.

19. The method of any one of embodiments 16-18, wherein the method also provides location information for one or more target(s) within the substantially 2D sample.

20. The method of any one of embodiments 16-19, which includes the enzymatic activity of one or more of RNase H, DNA polymerase, reverse transcriptase, RNA polymerase, and/or one or more restriction enzyme(s).

21. The method of any one of embodiments 16-20, further including sequence analysis of a plurality of nucleic acid molecules containing one or molecular ID tag(s).

22. A spatially-encoded capture array, substantially as described or illustrated herein.

23. The spatially-encoding capture array of embodiment 22, as illustrated in FIG. 10 or FIG. 11, or in Appendix A of priority U.S. Provisional Application No. 63/487,575, filed on Feb. 28, 2023.

24. The spatially-encoding capture array of embodiment 22 or 23, including capture elements embedded in a biomolecule-permeable matrix.

25. The spatially-encoded capture array of embodiment 24, wherein: the capture elements include beads; the biomolecule-permeable matrix is a gel; or both.

26. The spatially-encoded capture array of embodiment 24 or embodiment 25, in the form of a pliable “sticker” intended to be used in direct contact with a substantially 2D sample, for analysis of targets within that sample.

27. A spatial encoding workflow essentially as described or illustrated herein.

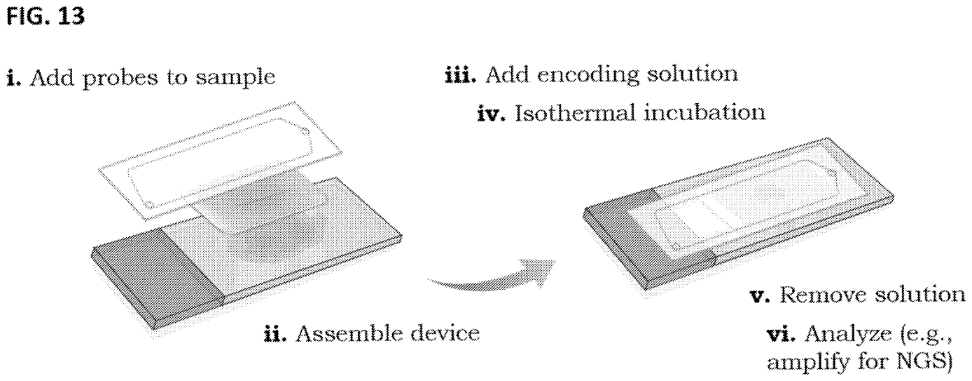

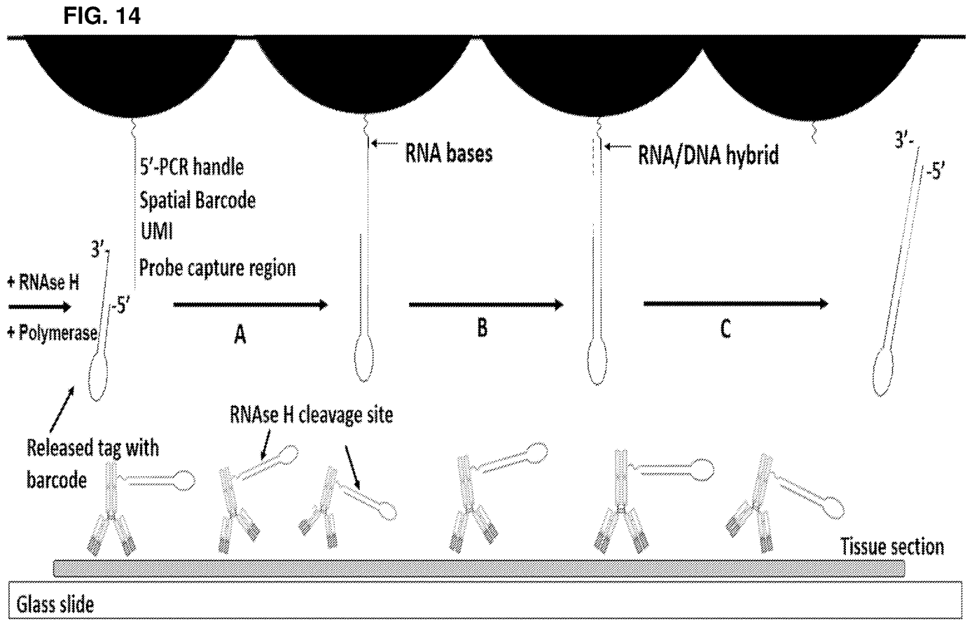

28. The spatial encoding workflow of embodiment 27, as illustrated in FIG. 12. or FIG. 13, or Appendix A of priority U.S. Provisional Application No. 63/487,575, filed on Feb. 28, 2023.

29. A spatially-encoding feature array having the grid within a grid arrangement illustrated in FIG. 10 and describe herein.

30. A computer readable medium or digital resource, or a digital database, containing spatial location information for features of a spatially-encoding array as describe herein.

31. The computer readable medium or digital resource, or a digital database, of embodiment 30, which contains spatial location information for substantially all the features of the spatially-encoding array.

32. Use of the computer readable medium or digital resource, or a digital database, of embodiment 30 or embodiment 31, to provide a user with location information for one or more targets correlated with the spatial location information of the spatially-encoding array.

33. The use of embodiment 32, wherein the correlation arises through use of the spatially-encoding array in a workflow or method as described or illustrated herein.

34. A method for isothermal spatial encoding of biological samples, substantially as described or illustrated herein.

35. Use of a DNA/RNA hybrid hairpin tag as described or illustrated herein, for transcriptomic analysis of a biological sample.

36. A spatial encoding surface, such as a capture array, for instance a bead-based capture array, substantially as describe or illustrated herein.

### Exemplary Embodiments Set 2

1. A Hairpin Tag nucleic acid molecule including, functionally connected in 5′ to 3′ order, parts A-B-C-D-E, wherein: part A includes a Cleavable Site including either: (1) a string of RNA bases, or (2) a string of DNA bases including a restriction enzyme (RE) recognition site; part B includes a string of DNA bases including a Tag PCR Handle; part C includes a string of DNA bases including a Tag ID Barcode, and which string of DNA bases forms a portion of the loop of the hairpin; part D includes a string of DNA bases having the reverse complementary sequence of the Tag PCR Handle, thereby forming a portion of the stem of the hairpin; and part E includes a string of DNA bases having the reverse complementary sequence of at least a portion of either (1) the string of RNA bases of part A or (2) the string of DNA bases including the RE recognition site, thereby forming a portion of the stem of the hairpin.

2. The Hairpin Tag nucleic acid molecule of embodiment 1, further including an attachment moiety conjugated to the 5′ end of part A.

3. The Hairpin Tag nucleic acid molecule of embodiment 2, wherein the attachment moiety provides amine-reactive crosslinker activity or thiol-reactive crosslinker activity.

4. The Hairpin Tag nucleic acid molecule of embodiment 2, further including a linker between the attachment moiety and part A.

5. The Hairpin Tag nucleic acid molecule of embodiment 4, wherein the linker includes PEG(n), where n=1-20.

6. The Hairpin Tag nucleic acid molecule of embodiment 1, wherein the Tag ID Barcode is at least 4 bases long.

7. The Hairpin Tag nucleic acid molecule of embodiment 6, wherein the Tag ID Barcode is 4, 5, 6, 7, 8, or more than 8 bases long.

8. The Hairpin Tag nucleic acid molecule of embodiment 1, wherein the Tag PCR Handle in part B and the reverse complement thereof in part D are each at least 10 bases long.

9. The Hairpin Tag nucleic acid molecule of embodiment 8, wherein the Tag PCR Handle in part B and the reverse complement thereof in part D are each 11, 12, 13, 14, 15, 16, 17, 18, 19, 20, or more than 20 bases long.

10. The Hairpin Tag nucleic acid molecule of any one of embodiments 1-9, which is constructed at least in part using a templated ligation reaction.

11. The Hairpin Tag nucleic acid molecule of any one of embodiments 1-10, wherein part A includes the string of DNA bases including the RE recognition site, and RE recognition site is at least 4 bases long.

12. The Hairpin Tag nucleic acid molecule of embodiment 11, wherein the RE recognition site is 4, 5, 6, 7, 8, 9, 10, 11, 12, 13, 14, or longer than 14 bases long.

13. The Hairpin Tag nucleic acid molecule of embodiment 11, wherein the RE recognition site is a Type IIS Restriction Enzyme recognition site, a site for a RE that produces a 3′ overhang, or both.

14. The Hairpin Tag nucleic acid molecule of any one of embodiments 1-10 which is a DNA/RNA Hybrid Hairpin Tag nucleic acid molecule, and wherein part A includes the string of RNA bases, and the string of RNA bases in part A is at least 5 bases long.

15. The DNA/RNA Hybrid Hairpin Tag nucleic acid molecule of embodiment 14, wherein the string of RNA bases in part A is 5, 6, 7, 8, 9, 10, 11, 12, 13, 14, 15, 16, 17, 18, 19, 20, or longer than 20 bases long.

16. A set of two or more Hairpin Tag nucleic acid molecules of any one of embodiments 1-13 and/or DNA/RNA Hybrid Hairpin Tag nucleic acid molecules of embodiment 14 or embodiment 15, where each of the two or more nucleic acid molecules has a unique Tag ID Barcode sequence.

17. The set of two or more Hairpin Tag nucleic acid molecules and/or DNA/RNA Hybrid Hairpin Tag nucleic acid molecules of embodiment 16, further including at least one Attenuation Tag-like nucleic acid molecule, which Attenuation Tag-like nucleic acid molecule differs from a Hairpin Tag nucleic acid molecule or DNA/RNA Hybrid Hairpin Tag nucleic acid molecule of the set by lack of a functional Cleavable Site.

18. The set of two or more Hairpin Tag nucleic acid molecules and/or DNA/RNA Hybrid Hairpin Tag nucleic acid molecules of embodiment 17, wherein the Attenuation Tag-like nucleic acid molecule differs from a Hairpin Tag nucleic acid molecule or DNA/RNA Hybrid Hairpin Tag nucleic acid molecules of the set by (1) having DNA bases in place of the RNA bases of part A, or (2) lacks the RE recognition site of part A.

19. A Tagged Probe, including a Probe Molecule to which is attached through the attachment moiety to a Hairpin Tag nucleic acid molecule of any one of embodiments 2-13 or to a DNA/RNA Hybrid Hairpin Tag nucleic acid molecule of embodiment 14 or embodiment 15.

20. The Tagged Probe of embodiment 19, wherein the Probe Molecule includes an affinity molecule having a binding affinity for a target molecule.

21. The Tagged Probe of embodiment 20, wherein the affinity molecule includes an antibody binding domain having affinity for an antigen, and the target molecule includes the antigen.

22. The Tagged Probe of embodiment 19, wherein the probe molecule includes one or more of: an antibody or binding fragment thereof, a nucleic acid, a small molecule, an organic or inorganic chemical, a putative drug target, an identified pharmaceutical drug, or a biological macromolecular complex.

23. The Tagged Probe of embodiment 19, wherein the probe molecule is one of a set of probe molecules, each of which includes one of a plurality of members of a library of small molecules, a library of drug targets, a library of biological affinity molecules, a library of natural products, a library of bio-active compounds, a genomic library, a transcriptomic library, a metabolomic library, or a drug screening library.

24. The Tagged Probe of embodiment 20, wherein the target molecule includes a biological molecule, an inorganic object, or an addressable feature of an array.

25. The Tagged Probe of embodiment 24, wherein the target molecule includes a biological molecule, and the biological molecule includes one or more of a protein, lipid, carbohydrate, a nucleic acid molecule, or a combination of proteins, lipids, carbohydrates and/or nucleic acid molecules.

26. The Tagged Probe of embodiment 25 wherein the target molecule is one of a plurality of molecules making up a complex, and the complex is located outside of or within a cell or cells in a tissue sample.

27. The Tagged Probe of any one of embodiments 19-26, further including an amplification sequence including a polymerase promoter sequence.

28. The Tagged Probe of embodiment 27, wherein the amplification sequence includes a T7 promoter sequence, such as a T7 promoter adaptor.

29. A Released Hairpin Tag nucleic acid molecule derived from a Hairpin Tag of any one of embodiments 2-13, or from a DNA/RNA Hybrid Hairpin Tag of embodiment 14 or embodiment 15, wherein the Released Hairpin Tag has been separated from the attachment moiety by enzymatic action of a Restriction Endonuclease or a RNase H enzyme.

30. A Capture Oligo (CO) nucleic acid molecule including, functionally connected in 5′ to 3′ order, parts 1-II-III-IV, wherein: part I includes a Cleavable Site including either: (1) a single RNA base or a contiguous string of RNA bases, or (2) a string of DNA bases including a restriction enzyme (RE) recognition site; part II includes a string of DNA bases including a CO PCR Handle; part III includes a string of DNA bases including a Spatial Barcode; and part IV includes a string of DNA bases including a Tag Capture Region.

31. The CO nucleic acid molecule of embodiment 30, further including an attachment moiety conjugated to the 5′ end of part 1.

32. The CO nucleic acid molecule of embodiment 31, wherein the attachment moiety provides amine-reactive crosslinker activity or thiol-reactive crosslinker activity.

33. The CO nucleic acid molecule of embodiment 31, further including a linker between the attachment moiety and part 1.

34. The CO nucleic acid molecule of embodiment 33, wherein the linker includes PEG(n), where n=1-20.

35. The CO nucleic acid molecule of embodiment 30, wherein the Spatial Barcode is at least 4 bases long.

36. The CO nucleic acid molecule of embodiment 35, wherein the Spatial Barcode is 4, 5, 6, 7, 8, or more than 8 bases long.

37. The CO nucleic acid molecule of embodiment 30, wherein the CO PCR Handle in part II is at least 5 bases long.

38. The CO nucleic acid molecule of embodiment 37, wherein the CO PCR Handle in part II is 5, 6, 7, 8, 9, 10, 11, 12, 13, 14, 15, 16, 17, 18, 19, 20, or longer than 20 bases long.

39. The CO nucleic acid molecule of any one of embodiments 30-38, further including a unique molecular identifier (UMI).

40. The CO nucleic acid molecule of any one of embodiments 30-38, the sequence of which has no more than 2 contiguous bases of internal sequence self-complementarity.

41. The CO nucleic acid molecule of embodiment 40, the sequence of which has no more than more than 3, no more than 4, no more than 5, no more than 6, no more than 7, no more than 8, no more than 9, no more than 10, no more than 11, or no more than 12 contiguous bases of internal sequence self-complementarity.

42. The CO nucleic acid molecule of any one of embodiments 30-41, which is constructed at least in part using a templated ligation reaction.

43. The CO nucleic acid molecule of any one of embodiments 30-42, wherein part I includes the string of DNA bases including the RE recognition site, and RE recognition site is at least 4 bases long.

44. The CO nucleic acid molecule of embodiment 43, wherein the RE recognition site is 4, 5, 6, 7, 8, 9, 10, 11, 12, 13, 14, or longer than 14 bases long.

45. The CO nucleic acid molecule of embodiment 43, wherein the RE recognition site is a Type IIS Restriction Enzyme recognition site, a site for a RE that leaves a 3′ overhang, or both.

46. The CO nucleic acid molecule of any one of embodiments 30-42 which is a DNA/RNA chimeric CO nucleic acid molecule, and wherein part I includes the single RNA base.

47. The CO nucleic acid molecule of any one of embodiments 30-42 which is a DNA/RNA chimeric CO nucleic acid molecule, and wherein part I includes the contiguous string of RNA bases.

48. The DNA/RNA chimeric CO nucleic acid molecule of embodiment 47, wherein the contiguous string of RNA bases in part I is 2, 3, 4, 5, 6, 7, 8, 9, 10, 11, 12, 13, 14, 15, 16, 17, 18, 19, 20 bases long, or longer than 20 bases long.

49. A set of two or more CO nucleic acid molecules of any one of embodiments 30-45 and/or DNA/RNA chimeric CO nucleic acid molecules of any one of embodiments 46-48, where each of the two or more nucleic acid molecules has a unique Spatial Barcode sequence.

50. A CO nucleic acid molecule of any one of embodiments 31-45 or a DNA/RNA chimeric CO nucleic acid molecule of any one of embodiments 46-48, which is attached to a Capture Feature through the attachment moiety.

51. The CO nucleic acid molecule of embodiment 50, wherein the Capture Feature is a bead, a chemically-functionalized spot on a glass surface, a defined region of a chemically functionalized and permeable gel, a bead or other inorganic object embedded within or on the surface of a permeable gel, or one of a series of spatially defined objects attached to a gel.

52. The CO nucleic acid molecule of embodiment 50, wherein the Capture Feature is a spatially addressable feature in an array.

53. The CO nucleic acid molecule of embodiment 52, wherein the array is a microarray having at least 100 addressable Capture Features.

54. A Spatially Encoded Capture Feature, including a Capture Feature to which is attached through the attachment moiety a CO nucleic acid molecule of any one of embodiments 31-42 or a DNA/RNA chimeric CO nucleic acid molecule of any one of embodiments 43-48.

55. The Spatially Encoded Capture Feature of embodiment 54, wherein the Capture Feature includes a bead or an addressable location on a substantially 2-dimensional surface.

56. The Spatially Encoded Capture Feature of embodiment 54 or embodiment 55, which is one Spatially Encoded Capture Feature within an array of at least 100 different Spatially Encoded Capture Features, and wherein the CO nucleic acid molecule on each of the at least 100 different Spatially Encoded Capture Features of the array each include a different Spatial Barcode.

57. A Capture Pair, including: a Hairpin Tag nucleic acid molecule of any one of embodiments 2-13, or a DNA/RNA Hybrid Hairpin Tag nucleic acid molecule of embodiment 14 or embodiment 15, or a Released hairpin tag nucleic acid molecule of embodiment 29; and a Capture Oligo (CO) nucleic acid molecule of any one of embodiments 31-42 or a DNA/RNA chimeric CO nucleic acid molecule of any one of embodiments 43-48, or an Attached CO of any one of embodiments 50-73, wherein the sequence of the Hairpin Tag nucleic acid molecule and the sequence of the CO nucleic acid molecule are at least partially complementary, such that when the Hairpin Tag nucleic acid molecule is released from its attachment moiety in proximity to the CO nucleic acid molecule, the released Hairpin Tag nucleic acid molecule is captured by sequence complementarity bonding at the 3′ ends to the CO nucleic acid molecule, such that resultant complex of the released Hairpin Tag nucleic acid molecule and the CO nucleic acid molecule is competent for a downstream extension reaction by a polymerase enzyme.

58. A Spatially Encoding Capture Array, including a defined array of spatially-addressed capture features, wherein each capture feature includes: a spatially-identifiable feature including: a pre-defined, addressable location on a substantially two-dimensional solid surface; or a bead or other similar separate, solid capture object; and attached at each feature, multiple copies of a CO nucleic acid molecule of any one of embodiments 31-42 or a DNA/RNA chimeric CO nucleic acid molecule of any one of embodiments 43-48, wherein the CO nucleic acid molecules at each feature have a unique Spatial Barcode sequence compared to the CO nucleic acid molecules at other features in the array.

59. The Spatially Encoding Capture Array of embodiment 58, wherein one or more CO nucleic acid molecule(s) is applied to spatially-addressed capture feature(s) in the array by: droplet printing of the CO into the pre-defined, addressable locations on the substantially two-dimensional solid surface, or attachment of the CO onto beads through the attachment moiety.

60. The Spatially Encoding Capture Array of embodiment 58 or embodiment 59, which array includes beads each of which include a Visual Barcode operationally coupled to the COs.

61. The Spatially Encoding Capture Array of embodiment 60, wherein the Visual Barcode enables beads to be assigned to locations within in the capture array.

62. The Spatially Encoding Capture Array of embodiment 58, wherein the bead or other similar, separate capture objects are embedded in a biomolecule-permeable matrix.

63. The Spatially Encoding Capture Array of embodiment 62, wherein: the capture objects include beads; the biomolecule-permeable matrix includes a gel; or both.

64. The Spatially Encoding Capture Array of embodiment 63, wherein the biomolecule-permeable matrix that includes the gel is formatted as a pliable sticker.

65. The Spatially Encoding Capture Array of embodiment 62, wherein the biomolecule-permeable matrix: is structurally stable at a selected temperature between 4-45° C.; is permeable to proteins such as functional RNAseH and polymerase; is permeable to ribonucleoside tri-phosphates (rNTPs) and/or deoxynucleotide triphosphates (dNTPs); is permeable to Mg²⁺ ions; is substantially inert to biological molecules; and is sufficiently flexible to permit application of a relatively thin layer of the matrix directly to a substantially two-dimensional sample or surface.

66. The Spatially Encoding Capture Array of embodiment 65, which is formulated as a three-dimensional thin-layer gel, the width and length of which are substantially larger than its thickness, and wherein the spatially-identifiable capture features are arranged substantially in a single plane across surface of the gel defined by its the length and width.

67. The Spatially Encoding Capture Array of embodiment 66, in which at least a first of the spatially-identifiable capture features is immediately adjunct and/or touching a second of the spatially-identifiable capture features.

68. The Spatially Encoding Capture Array of any one of embodiments 62-67, wherein matrix includes a hydrogel or polyacrylamide gel.

69. The Spatially Encoding Capture Array of embodiment 68, wherein the thickness of the matrix or gel is no more than about 2 mm.

70. The Spatially Encoding Capture Array of embodiment of embodiment 69, wherein the thickness of the matrix or gel is no more than about 1 mm, no more than 500 μm, no more than 250 μm, no more than 200 μm, no more than 150 μm, no more than 125 μm, no more than 100 μm, or less than 100 μm.

71. The Spatially Encoding Capture Array of embodiment of embodiment 70, wherein the thickness of the matrix or gel is 100-200 μm, 100-150 μm, or about 125 μm.

72. The Spatially Encoding Capture Array of any one of embodiments 58-71, which array is reinforced by an inert mesh or other support structure.

73. The Spatially Encoding Capture Array of any one of embodiments 58-72, wherein: the pre-defined, addressable location on a substantially two-dimensional solid surface has a surface area of no more than about 1 μm×1 μm; or the bead or other similar separate, solid capture object has diameter of no more than about 20 μm.

74. The Spatially Encoding Capture Array of embodiment 73, wherein the bead or other similar separate, solid capture object has diameter of no more than 18 μm, no more than 15 μm, no more than 12 μm, no more than 10 μm, no more than 8 μm, no more than 5 μm, no more than 3 μm, no more than 1 μm, or about 100 nm.

75. The Spatially Encoding Capture Array of embodiment 74, wherein the bead or other similar separate, solid capture object has diameter of between 1-3 μm.

76. A semi-ordered Spatially Encoding Capture Array, including: a grid of spatially addressable locations, each of which is labeled with oligonucleotides having a unique X-Y coordinated sequence, which oligonucleotides are applied to the spatially addressable locations by splint ligation of: a x-coordinate adapter oligonucleotide, which x-coordinate adapter oligonucleotide is used to label all spatially addressable locations within a row of the grid; and a y-coordinate adapter oligonucleotide, which y-coordinate adapter oligonucleotide is used to label all spatially addressable locations within a column of the grid.

77. The semi-ordered Spatially Encoding Capture Array of embodiment 76, which is constructed at least in part using a method provided in FIGS. 17A-17C.

78. The semi-ordered Spatially Encoding Capture Array of embodiment 76, which is has a grid-within-a-Grid format as illustrated in FIG. 10.

79. A semi-ordered Spatially Encoding Capture Array, including: an array of uniquely identifiable capture features, which array includes two or more sub-arrays, each sub-array including uniquely identifiable capture features the location of which is specified at least in part by identification of the sub-array within the semi-ordered Spatially Encoding Capture Array.

80. A semi-ordered Spatially Encoding Capture Array, including: a set of two or more sub-arrays each including a plurality of capture features, in which each capture feature within each sub-array is attached to a capture oligo including a unique Location Tag, the capture features within each sub-array are randomly arranged, and the capture features within each sub-array further include a sub-array-identifying oligo tag attached by splint ligation to the capture oligos on each feature in the sub-array.

81. A method for detection and/or quantification and/or localization of targets in a substantially two-dimensional (2D) sample, the method including: contacting a substantially 2D sample with at least one tagged probe to produce a substantially 2D stained sample, which tagged probe includes: a Hairpin Tag nucleic acid molecule including an attachment moiety and a Tag ID Barcode, or a DNA/RNA Hybrid Hairpin Tag nucleic acid molecule including an attachment moiety and a Tag ID Barcode; and a Probe Molecule attached through the attachment moiety to the Hairpin Tag nucleic acid molecule including a Tag ID Barcode, or the DNA/RNA Hybrid Hairpin Tag nucleic acid molecule including a Tag ID Barcode; contacting a surface of the substantially 2D stained sample with a permeable, spatially encoding capture array to form a sample-array sandwich, which spatially encoding capture array includes: a plurality of spatially identifiable features; and attached at each spatially identifiable feature, multiple copies of a Capture Oligo (CO) nucleic acid molecule or a DNA/RNA chimeric CO nucleic acid molecule, wherein the CO nucleic acid molecules at each feature have a unique Spatial Barcode sequence compared to the CO nucleic acid molecules at other features in the array; placing a flow cell or other solution-containing cover over the sample-array sandwich to form an enclosure containing the sample-array sandwich; adding to the enclosure a solution including reaction components to form a reaction mixture, which components include: a cleavage enzyme selected from a RNAseH or at least one restriction endonuclease (RE); at least one polymerase; a mixture of ribonucleoside tri-phosphates (rNTPs) and/or deoxynucleotide triphosphates (dNTPs); Mg²⁺ ions; and a buffering agent; incubating the sample-array sandwich in contact with the reaction mixture at an assay temperature for 30-60 minutes, to form a reaction product mixture; removing at least a portion of the reaction product mixture from the enclosure; and analyzing the reaction product mixture to detect and/or quantify and/or define the location of targets in the substantially 2D sample.

82. The method for detection and/or quantification and/or localization of targets in a substantially two-dimensional (2D) sample of embodiment 81, wherein the enclosure includes a flow cell.

83. The method of embodiment 81 or embodiment 82, which is carried out at a single temperature (isothermally) or within a range of about 5° C. within a single temperature.

84. The method of any one of embodiments 81-83, wherein the assay temperature has a range of 20-55° C., or 37-42° C.

85. The method of any one of embodiments 81-84, wherein the method provides location information for more than one target within the substantially 2D sample.

86. The method of any one of embodiments 81-85, wherein the at least one polymerase provides an enzymatic activity of DNA polymerase, reverse transcriptase, or RNA polymerase.

87. The method of any one of embodiments 81-86, wherein analyzing the reaction product mixture includes sequence analysis of a plurality of nucleic acid molecules containing a Spatial Barcode and a Tag ID Barcode.

88. The method of embodiment 87, wherein analyzing the reaction product mixture includes next generation sequencing (NGS) of a plurality of nucleic acid molecules containing a Spatial Barcode and a Tag ID Barcode.

89. The method of embodiment 87 or embodiment 88, wherein the plurality of nucleic acid molecules containing a Spatial Barcode and a Tag ID Barcode are full extension products released from the spatially identifiable features by cleavage at the Cleavage Site of the CO.

90. The method of embodiment 89, wherein the cleavage includes RNAseH enzymatic activity or restriction endonuclease activity.

91. The method of any one of embodiments 81-90, wherein analyzing includes assigning a spatial location of at least one nucleic acid molecule containing a Spatial Barcode and a Tag ID Barcode within the substantially 2D sample.

92. A method for isothermal spatial encoding of a biological sample, including the method of any one of embodiments 81-91, wherein the substantially 2D sample is a biological sample.

93. Use of the set of Hairpin Tag nucleic acid molecules of any one of embodiments 16-18 or the set of Capture Oligo (CO) nucleic acid molecules of embodiment 49, or both, for transcriptomic analysis of a biological sample.

94. A spatial encoding surface, such as a capture array, for instance a bead-based capture array, substantially as described or illustrated herein.

95. A spatial encoding workflow, including: contacting a hybrid RNA/DNA tag including a spatial barcode conjugated to an antibody probe with a substantially two-dimensional (2D) tissue sample, to produce a stained sample; placing a capture array including capture features in contact with the stained sample to produce a sample/array sandwich; placing a fluid-containment enclosure on top of the sample/array sandwich; introducing assay solution including active RNAseH and active polymerase into the fluid-containment enclosure, thereby bringing the assay solution into contact with the sample/array sandwich; incubating the sample/array sandwich in the sample solution at a temperature and for a period of time sufficient for the RNAseH activity to at least partially digest the hybrid RNA/DNA tag to produce a cleaved tag and thereby releasing the cleaved tag into the assay solution in proximity to a capture feature; permitting interaction of the cleaved tag with a capture oligo (CO) on the proximal capture feature to provide a captured cleaved tag; incubating the sample/array sandwich in the sample solution at a temperature and for a period of time sufficient for the polymerase activity to extend the captured cleaved tag using the CO as template, to produce an extension product; cleaving the extension product, upon sufficient extension to produce a complementary RNA/DNA region based on the RNA base(s) in the CO, with RNAseH in the sample solution, thereby releasing the full-length extension products; collecting at least a portion of the full-length, released extension products; and amplifying and/or sequencing at least one of the full-length, released extension products.

96. A computer readable medium or digital resource, or a digital database, containing spatial location information for features of a spatially encoding array of any one of embodiments 58-80.

97. The computer readable medium or digital resource, or a digital database, of embodiment 96, which contains spatial location information for substantially all the features of the spatially encoding array.

98. Use of the computer readable medium or digital resource, or a digital database, of embodiment 96 or embodiment 97, to provide a user with location information for one or more targets correlated with the spatial location information of the spatially encoding array.

99. The use of embodiment 98, wherein the correlation arises through use of the spatially encoding array in a workflow or method as described or illustrated herein.

100. A kit, including one or more of: two or more Hairpin Tag nucleic acid molecules of any one of embodiments 1-13; two or more DNA/RNA Hybrid Hairpin Tag nucleic acid molecules of embodiment 14 or embodiment 15; the set of two or more Hairpin Tags of any one of embodiments 16-18; a set of two or more Tagged Probes of any one of embodiments 19-28; two or more Capture Oligo (CO) nucleic acid molecules of any one of embodiments 30-46; two or more DNA/RNA chimeric CO nucleic acid molecules of embodiment 47 or embodiment 48; the set of two or more CO nucleic acid molecules of any one of embodiments 30-45; the set of two or more DNA/RNA chimeric CO nucleic acid molecules of any one of embodiments 46-48; two or more CO nucleic acid molecules, each attached to a capture feature, of any one of embodiments 50-53; at least one spatially encoding capture array of any one of embodiments 58-75; at least one semi-ordered spatially encoding capture array of any one of embodiments 76-80; or the spatial encoding surface of embodiment 94.

101. The kit of embodiment 100, wherein the spatially encoding capture array, semi-ordered spatially encoding capture array, or spatial encoding surface is in the format of a pliable sticker.

102. The kit of embodiment 100, further including one or more of: a container in which is contained functional RNAseH enzyme; a container in which is contained functional polymerase enzyme; a container in which is contained functional restriction enzyme; a container in which is contained one or a mixture of ribonucleoside tri-phosphates (rNTPs) and/or deoxynucleotide triphosphates (dNTPs); a container in which is contained a solution including Mg²⁺ ions; or a container in which is contained a buffer solution.

103. The kit of embodiment 101, wherein at least one of the containers contains at least two of the RNAseH enzyme, the polymerase enzyme, the restriction endonuclease enzyme, the rNTPs and/or dNTPs, the Mg²⁺ ions, or the buffer solution.

104. The kit of any one of embodiments 100-103, further including one or more of: components useful for preparing a sample for analysis using a method provided herein; a solution-containing cover suitable for placement over a sample-array sandwich on a slide, in order to form a fluid-containing enclosure for the sample-array sandwich; a flow cell cover; or a glass slide or other surface suitable for receiving a substantially two-dimensional sample.

### Example 1: Construction of DNA/RNA Hybrid Hairpin Tags

Exemplary DNA/RNA hybrid hairpin tags were constructed by ligation of two separate nucleic acids; a first nucleic containing RNA bases within the 5′ region (tag stem oligo), and a second nucleic acid made entirely of DNA, containing bases at the 3′-end designed for complementarity with the RNA bases of the first nucleic acid, followed by a hairpin loop region, and a stem region at the 5′-end (tag loop oligo). In all cases, the first nucleic acid (tag stem oligo) contained a 5′ flexible linker connecting the nucleic acid to a reactive primary amine (Integrated DNA Technologies (IDT): /5AmMC6/or IDT: /5AmMC12/), and in some cases (as outlined below), the second nucleic acid (tag loop oligo) contained an internal amino-modified base (IDT: /iAmMC6T/) for covalent labeling with amine-reactive chemistries. Prior to labeling of nucleic acids, all remaining free amino groups carried over from the synthesis reaction (when ordering standard desalted oligos from the vendor) were removed by sodium acetate/EtOH precipitation using published protocols. Precipitated amino-modified oligos (and any internal amino-modified oligos, or oligos ordered as pre-purified arrived as lyophilized by the manufacturer), were resuspended to 400 μM in H₂O and then diluted 1:1 with 200 mM Sodium Phosphate buffer, pH 8.5. The resulting samples contained 200 μM of each amino-modified oligo in a final N-hydroxysuccinimide (NHS) conjugation buffer composition of 100 mM Sodium Phosphate, pH 8.5 for downstream labeling with NHS-modified reagents.

To each 100 μl oligo sample, 10 μl of an appropriate NHS-bearing labeling reagent (at 20 mM in anhydrous DMF) was added at about a 10-fold molar excess for overnight conjugation at room temperature. Oligo labeling reactions were then quenched by adding 25 μl of 1M Tris pH 7.5 to each sample. All oligo labeling reactions were analyzed by 15% TBU gel (ThermoFisher EC6885BOX), whereby unmodified oligos of the same type were ran side-by-side with modified oligos in adjacent lanes to observe a clear upward shift of bands within lanes corresponding to their respective successful and complete modification with the appropriate NHS reagent (no bands corresponding to unmodified oligos were observed in any of the modified oligo lanes); 2 pmol loaded per band, stained with SYBR™ Gold (ThermoFisher S11494). To remove excess labeling reagent from each reaction, oligos were precipitated using sodium acetate/EtOH, resuspended in 100 μl of H₂O, then desalted using a 0.5 ml 7.5K MWCO Zeba™ column (ThermoFisher 89883) equilibrated with H₂O to further remove excess labeling reagents. Oligos labeled with surface or probe attachment chemistries (biotin or trans-cyclooctene (TCO) used here) were subjected to a second desalting step by repeating the above desalting procedure for more complete removal of any remaining free label. Oligos labeled with fluorophores were generally subjected to a single desalting step. These two oligo species (the tag stem oligo and the tag loop oligo) were covalently linked together enzymatically, using either T4 DNA ligase (M0202S or SplintR® ligase (NEB M0375S) with relatively equivalent efficiencies as determined by denaturing nucleic acid gel analysis using 10% TBU gels (ThermoFisher EC68752BOX).

Different versions of DNA/RNA hybrid hairpin tags (referred to here as v1-v6) were tested for comparison of their relative performance when conjugated to surfaces and to antibody probes, where they were used in analyte binding experiments and in tissue staining workflows. From these experiments it was concluded that smaller and more compact tag versions containing fewer unpaired bases within their stem and loop regions showed fewer non-specific interactions with tissue samples under optimized staining procedures. Due to its compact size (and relatively few single stranded bases exposed to solvent, which could be responsible for generating non-specific interactions with the sample), and its overall performance in tissue staining procedures, tag version v6 was chosen for downstream experiments. Tag version v6 (the full sequence of which is provided herein) can be ordered as a full-length tag from a vendor (purchased from IDT here), to contain a 5′ flexible linker bearing a primary amino group (IDT: /5AmMC12/) for conjugation with NHS-bearing labeling agents, without the need for ligation of two separate species. However, for all dual-labeled v6 tags used here, the described two-step labeling and ligation procedure was employed prior to gel purification of ligation products.

### Example 2: Optimization of the Tag Capture Region

A series of oligos was designed and ordered from IDT (Ab_DRD-Tag1 cleaved v1-v6, shown below) for determining optimal capture of cleaved hairpin tags by the capture oligo. These capture experiments were conducted at the isothermal assay temperature (42° C.), and within the isothermal assay buffer (1× RNAse H buffer from NEB). Oligos within this series of hairpins differ only by the length of their capturable regions, enabling a titration series around the capture Tm. The underlined sequence corresponds to the region of each oligo in the series that was captured by the capture oligo in the experiment. The salt adjusted Tm of these sequences was calculated using the Northwestern Oligo Calc website, analyzed at an oligo concentration of 50 nM and salt (monovalent cation: Na+) concentration of 75 mM.
Ab_DRD-Tag1 cleaved v1; salt adjusted Tm=60.4 (SEQ ID NO: 8)
Ab_DRD-Tag1 cleaved v2; salt adjusted Tm=54.3 (SEQ ID NO: 9)
Ab_DRD-Tag1 cleaved v3; salt adjusted Tm=52.9 (SEQ ID NO: 10)
Ab_DRD-Tag1 cleaved v4; salt adjusted Tm=46.4 (SEQ ID NO: 11)
Ab_DRD-Tag1 cleaved v5; salt adjusted Tm=43.8 (SEQ ID NO: 12)
Ab_DRD-Tag1 cleaved v6; salt adjusted Tm=40.9 (SEQ ID NO: 13)

To perform the capture experiments, MyOne T1 Streptavidin Dynabeads were coated with a single, biotin-modified capture oligo type consisting of all DNA bases (DRD1_CO_6sUMI—DNA bases; SEQ ID NO: 14) while mixing at room temperature in SA bind buffer (10 mM Tris pH 7.5, 500 mM NaCl), at a bead concentration of 1 mg/mL and an oligo concentration of 200 nM. Capture beads were washed as outlined above, and 1 mg/ml beads were mixed together with the various hairpin tags (cleaved v1-v6; SEQ ID NOs: 8-13) at a hairpin tag concentration of 50 nM, for incubation in 1× RNAse H buffer (50 mM Tris-HCl, 75 mM KCl, 3 mM MgCl₂, 10 mM DTT (pH 8.3) for 30 minutes at 42° C. degrees. Following incubation, samples were moved rapidly from the temperature block to a magnetic tube stand and given 3-4 minutes to ensure complete pulling of beads by the magnet. The supernatant was collected, and beads were resuspended in and equal volume of 1× RNAse H buffer to that of the collected supernatant volume. An equal amount of these two fractions were run on a 10% TBU denaturing gel (ThermoFisher EC68752BOX) and the amount of the hairpin oligo contained within the bead fraction was compared to the amount of the hairpin oligo contained within the supernatant fraction to generate a ratio for each hairpin oligo tested. Gel analysis revealed that hairpin oligo v4 was the most efficiently captured of the six hairpins tested under these conditions, and therefore this capture region length and composition became the basis for design of the final capture length of the optimized v6 hairpin tag.

### Example 3: Capture Oligos

Capture oligos (CO) used here were composed entirely of DNA (DRD1_CO_6sUMI—DNA bases; SEQ ID NO: 14) or were designed to contain RNA bases (DRD1_CO_6sUMI—RNA bases; SEQ ID NO: 38) within their 5′-ends. Capture oligos contained flexible linkers at their 5′-ends bearing a primary amino group for downstream conjugation with NHS-modified labels. A 3′-inverted T base modification was included to cap (prevent extension of) CO 3′-ends in experiments shown here. CO in NHS conjugation buffer (200 μM oligos in 100 mM sodium phosphate buffer, pH 8.5) were modified with NHS-LC-Biotin (ThermoFisher 21336) at a 10-fold molar excess of labeling agent as described in the labeling procedure outlined above, and allowed to react overnight at room temperature. Conjugation reactions were then quenched with 1M Tris pH 7.5 as described above, precipitated with sodium acetate/EtOH using standard procedures, and precipitated pellets were resuspended in 100 μl H₂O. Resuspended oligos were then subjected to two back-to-back desalting steps per sample, using 0.5 mL 7.5 MWCO Zeba desalting columns (ThermoFisher 89883) to remove as much free biotin as possible. To construct CO containing 5′-RNA bases, two oligos were splint-ligated together using T4 DNA ligase and a splinting oligo designed to be complementary to both the 3′-end of the biotinylated oligo containing RNA bases, and the 5′-end of the downstream oligo fragment encoding the remainder of the capture oligo sequence. Following ligation, the full-length, biotinylated CO product containing 5′-RNA bases was gel purified for use in downstream immobilization experiments.

### Example 4: Capture Beads

Capture beads were generated by coating MyOne T1 streptavidin Dynabeads (ThermoFisher 65601) with CO. In brief, beads were washed as per the manufacturer's instructions, sonicated to reduce bead clumping, and resuspended to 1 mg/mL in SA bind buffer (500 mM NaCl, 10 mM Tris pH 7.5) with 200 nM biotinylated CO and tubes were rotated for continuous mixing at room temperature for 30 minutes. Once coated with CO, the beads were washed six times in SA bind buffer with sonication steps performed in between washes to remove any free oligos. Capture beads were then resuspended in an appropriate buffer (different buffers were used for different intended applications) to a concentration of 1-2 mg/ml, sonicated to reduce clumping, and used in various downstream tag capture experiments.

### Example 5: Conjugation of Antibody Probes with DNA/RNA Hybrid Hairpin Tags

Gel analysis of tag-conjugated antibodies usually consisted of three lanes of a 3-8% Tris acetate gel (ThermoFisher WG1602BOX) for each conjugation reaction: Lane 1 contained a protein size marker; Lane 2 contained 1 μg of the original unconjugated antibody; Lane 3 contained ~2-3 μg of the tag conjugation reaction mixture. In a typical result, there were two bands in Lane 2; one that migrated at ~65 kD relative to the protein size marker, which corresponds to the BSA carrier protein in the antibody solution, and the second migrated at ~150 kD, which corresponds to the unconjugated antibody. In Lane 3, there were consistently two major bands shifted to higher positions in the gel (relative to the migration of the original unconjugated antibody), which correspond to antibodies modified with either one tag molecule or two tag molecules, as well as a very minor band corresponding to the original unconjugated antibody (usually <5%), and the ~65 kD band for the carrier BSA. This typical result indicated successful conjugation of DNA/RNA hybrid hairpin tags to antibody probes, whereby the reaction solution contained mostly one or two tags per antibody molecule (as expected). For analysis of antibodies conjugated with fluorescently-labeled DNA/RNA hybrid hairpin tags, the gel was first imaged using a fluorescence gel scanner (ThermoFisher iBright Imaging System) using an appropriate excitation wavelength and emission filter for the respective fluorophore) before staining gels with Coomassie blue. Two fluorescent bands were typically observed in lanes loaded with the conjugation reaction, and following Coomassie blue staining, similar band patterns to those seen in non-fluorescent analytical gels of conjugates (described above) were observed.

### Example 6: In Vitro Binding Assays

To determine whether conjugation to DNA/RNA hybrid hairpin tags affected antibody binding to target analytes, an in vitro binding assay using an octet instrument (OctetRED 384, forteBIO, Division of Pall Life Sciences) was performed. Here, anti-human IgG (Jackson ImmunoResearch 109-005-003) was conjugated with DNA/RNA hybrid tag v1 (SEQ ID NO: 3) and used in experiments alongside the unconjugated antibody as well as a sample of the conjugated antibody that was pre-incubated with RNAse H (NEB M0297S). The unmodified antibody and both conjugate samples were tested in a 3-fold dilution series ranging from 18 μM to 74 nM for binding to biotinylated human IgG immobilized on high precision streptavidin-coated octet tips (SATORIUS—Octet® High Precision Streptavidin (SAX) Biosensors—18-5117). The procedure was carried out at 37° C. and 500 RPM, and included a 15 minute tip pre-equilibration step in equilibration buffer (10 mM Tris pH 7.4 supplemented with 80 mM NaCl), followed by a 1 minute analyte loading step in loading buffer (1×PBS supplemented with 100 nM biotinylated human IgG), followed by a 1 minute wash step in blocking buffer (50% Superblock/50% PBS supplemented with 1 mg/ml BSA), followed by a 10 minute association step (exposure to antibody samples at different concentrations in blocking buffer), followed by a 10 minute dissociation step. These results showed similar association kinetics between all three samples at all concentrations tested, but a slight reduction in the rate of dissociation for the conjugate was observed. It was postulated that the use of 10% DMSO in the dissociation step may increase the rate of dissociation for the conjugate, and this was also observed in experiments. It was postulated that the inclusion of a small amount of detergent (0.01-0.2% Tween-20) or increasing the salt concentration (to 300 mM NaCl) in the dissociation step may further increase the rate of dissociation for the conjugate. These components improved the dissociation rate of the conjugate to a lesser extent that what was observed with inclusion of 10% DMSO. This slower dissociation was only observed using DNA/RNA hybrid hairpin tag v1 (SEQ ID NO: 3), and may not be true for tag v6 (SEQ ID NO: 7).

### Example 7: DNA/RNA Hybrid Tag-Conjugated Antibody Stability

To test the stability of conjugates stored over several weeks at 4° C., Tag v6-conjugated antibodies (α-ER [EPR4007](Abcam: ab108398) and α-HER2/ErbB2 (R&D Systems: MAB1129) were stored in PBS with 0.25% BSA at 4° C. for 7 weeks. Unconjugated (1 μg per lane) and HPv6 conjugated antibodies (2-3 μg per lane) were ran in separate lanes on a 3-8% tris-acetate gel and visualized by Coomassie blue staining. Lanes containing the stored conjugated antibodies showed that a majority (>95%) of the antibodies were still stably conjugated to either one or two molecules of Tag v6 after 7 weeks.

### Example 8: Exemplary Antibody Staining Procedures

The following protocol was utilized for antibody staining on cells in 2D culture:
1) Cells fixed with 4% PFA for 10 minutes.
2) Permeabilization by incubation with 0.25% Triton-X in 1×PBS for 15 minutes at RT.
3) Block with Image-iT™ FX signal enhancer (Life Technologies, 136933) for 30 minutes at RT.
4) Block for 30 minutes at RT with SuperBlock™ (Life Technologies, 37580) containing BSA (50 mg/ml) and sheared salmon DNA (Thermo, AM9680) at a ratio of 93%:2%:5%.
5) Bind antibody-tag conjugates at 4° C. overnight or RT for 2-hours.
6) Wash out unbound antibody with three, 5-minute washes in PBST (0.02% Tween-20 in 1×PBS).

For FFPE tissue sections, the following steps were performed:
1) FFPE slides baked at 65° C. for 30 minutes
2) Wash: 3× washes in Xylene (5-minutes), 2× washes in 100% EtOH (5-minutes), 2× washes in 95% EtOH (3-minutes), 2× washes in 70% EtOH (3-minutes), and 2× washes in H₂O (3-minutes).
3) Antigen retrieval was carried out in Sodium Citrate buffer (pH 6.0) under high pressure at 110° C. for 15 minutes.
4) Following antigen retrieval, the procedure then continues from step 3 of the antibody staining procedure above.

The labelling antibodies were used at the following concentration:
α-ER [EPR4007](Abcam: ab108398)—used at 1:150
α-HER2/ErbB2 (R&D Systems: MAB1129)—used at 1:100

### Example 9: Imaging

All imaging was conducted on a Leica THUNDER widefield deconvolution microscope with one of four objectives (HC PL APO 20×0.8 NA, HCX PL FLUOTAR L 40×0.6NA CORR PH2, HC APO 40×1.25 NA GLYC CORR CS2, or HC PL APO 63×1.4 NA OIL). Excitation light sources were provided by a Lumencor SPECTRA X Light engine housing the following LED light sources (395, 440, 470, 510, 550, 640 and 750). Emission filters consisted of a quad cube (Ex: 375-407, 462-496, 542-566, 622-654; Dc: 415, 500, 572, 660; Em: 420-450, 506-532, 578-610, 666-724) and a Y7 cube (Ex: 672-748, Dc: 760, Em: 765-855). A final fast filter wheel with the following LP filters (440, 510, 590, 700, 100%) was used to clean up the left-hand side of the emission signal coming from the quad cube, before being collected on a Leica DFC9000 sCMOS camera.

### Example 10: Tag Retrieval and qPCR Analysis of Released Tags

In some experiments, DNA/RNA hybrid hairpin tags were cleaved from MyOne T1 Streptavidin Dynabeads (ThermoFisher), and in other experiments these tag types were cleaved from conjugated probes (antibodies) bound to analytes of biological samples immobilized on glass slides (for example, fixed cells or tissue sections). Following labeling of analytes within the samples (Her2 or ER were probed here) with the appropriate antibodies conjugated to two differentially-barcoded HPv6 tags (Her2-v6Tag1 and ER-v6Tag2, respectively), samples were briefly washed in 1× RNAse H buffer (50 mM Tris-HCl, 75 mM KCl, 3 mM MgCl₂, 10 mM DTT (pH 8.3). Following this wash, RNAse H (NEB M0297S) in 1× RNAse H buffer (75 Units of enzyme in 300 μl of buffer) was placed directly onto samples for tag cleavage (release). This reaction was performed on a heat block set to 37° C. for 30 minutes, and the solution was retrieved by aspiration from the sample (cells or tissue sections). The 300 μl aspirate was then split in half, with one half receiving capture beads (0.1 mg/mL final) coated with a capture oligo containing all-DNA bases (DRD1_CO_6sUMI—DNA bases; SEQ ID NO: 14). The other half (150 μl) received capture beads (0.1 mg/mL final) that were coated with a capture oligo containing RNA within their 5′-ends (DRD1_CO_6sUMI—RNA bases; SEQ ID NO: 38).
DRD1_CO_6sUMI—DNA bases (SEQ ID NO: 14)
DRD1_CO_6sUMI—RNA bases (SEQ ID NO: 38)

The DRD1_CO_6sUMI—RNA bases oligo was generated by splint-ligation of the following two smaller oligos:
CO_5_RNA (SEQ ID NO: 15)
CO_3_invT (SEQ ID NO: 16)
CO_Splint (used to ligate C05_RNA and CO_3_invT) (SEQ ID NO: 17)

To these two samples, dNTPs (200 μM each final) and 1 μl of Maxima Reverse Transcriptase (NEB) [200 U/μl], containing RNAse H activity were added for templated extension of the 3′-ends of captured tags by capture oligos. The extension reactions were placed at 37° C. for 45 minutes. Following this incubation, beads were pulled on a magnet, and the supernatant (~125 μl) from each of the two reactions was retrieved by aspiration. The remaining beads were resuspended in 125 μl of 1× RNAse H buffer and were placed at 85° C. for 3 minutes to melt extended product from the capture oligo. Samples were quickly moved from the heat source to the magnet and the supernatant was collected as soon as the beads were completely pulled by the magnet. These two fractions were labeled supernatant and beads, respectively for each sample. The fractions were then subjected to qPCR for quantification. In brief, 10 μl of the fractions were carried over to qPCR assays using Thermo Fisher Scientific PowerUp SYBR Master Mix with the following primers:
5′ DRD LNA v4 (SEQ ID NO: 18; wherein + denotes locked nucleic acid; LNA)
3′ DRD common primer v4 (SEQ ID NO: 19)

The first tests of tag retrieval, capture, extension, and amplification were conducted using Hpv6 antibodies conjugated to α-ER [EPR4007](ab108398) abcam or α-HER2/ErbB2 (MAB11129) that were used to stain a mixture of cultured SKBR3 (Her2+, ER−) and MCF-7 (Her2−, ER+) cells that were immobilized on glass slides.

### Example 11: A-HER2-v6Tag1 Antibody Conjugate Staining of Cultured SKBR3 vs. MCF7 Cells

For SKBR3 and MCF7 cell experiments, ~50,000 cells were grown on coverslips to ~70% cell density, fixed and permeabilized as described above, and used in downstream staining experiments. Capture beads were coated as described above, with one of two different capture oligo types for capture of released tags from probe-tag conjugates bound to biological samples: one containing all DNA bases (DRD1_CO_6sUMI—DNA bases; SEQ ID NO: 14) and the other containing RNA bases at the 5′-end (DRD1_CO_6sUMI—RNA bases; SEQ ID NO: 38). Inclusion of all DNA bases in the capture oligo enables capture, and extension of captured tag 3′-ends, while inclusion of RNA bases within the 5′-end of capture oligos enables capture, extension, and release of the extended products via the complete “one-pot isothermal” reaction, due to the generation of a new DNA/RNA hybrid region formed following tag extension by the polymerase. RNAse H activity of E. coli RNAse H (NEB M0297S) as well as RNAse H activity of the Maxima polymerase enzyme (ThermoFisher Maxima EP0741) can release tags from probes bound to the surface as well as extended tags bound to capture oligos via the newly formed DNA/RNA hybrid region. Following α-HER2-v6Tag1 staining experiments, samples were washed with 1× RNAse H buffer, and were exposed to a solution containing E. coli RNAse H, for cleaving tags from bound conjugates as described previously. This solution was removed from samples following incubation at the isothermal reaction temperature, split into two equal volumes, and capture beads of the two different types were added to capture the released α-HER2-v6Tag1 hairpins in the presence of Maxima RT and dNTPs. In experiments using capture oligos made of all DNA bases, qPCR results demonstrated about a 13.7-fold increase in released α-HER2-v6Tag1 hairpin levels within the bead fraction following probing of SKBR3 cells over the bead fraction following probing of the MCF7 cells with this conjugate, congruent with what is known about their respective expression levels of the HER2 protein. These results suggested that the tag cleavage (hairpin release), capture and extension steps of the procedure were working as expected.

Within probed SKBR3 cells, a 15-fold increase in released α-HER2-v6Tag1 hairpin signal was observed in the bead fraction over that of the supernatant fraction, and a 40-fold increase in signal was observed over the background signal by qPCR. Following experiments using capture oligos containing RNA bases with their 5′-ends, qPCR results demonstrated about a 11.7-fold increase in released α-HER2-v6Tag1 hairpin signal within the supernatant fraction following probing of SKBR3 cells with the anti-Her2-v6 tag conjugate over that of the supernatant fraction following probing of MCF7 cells. This retrieved signal was similar to the amount of signal obtained from the bead fraction when using the all-DNA capture oligo. Additionally, about a 1.5-fold increase in released α-HER2-v6Tag1 hairpin signal was observed within the supernatant fraction over that of the bead fraction, indicating that ~60% of the extended product was released into the supernatant following extension of the captured hairpin on the capture oligo. This result indicated that during the isothermal one pot information transfer reaction, the cleaved α-HER2-v6Tag1 hairpins were released from the bound probes, captured by the capture oligos containing 5′-RNA bases, the captured tags were then extended, and the extended products were then released from the capture beads as predicted. The extension product release step was then optimized in future experiments to obtain greater than 90% conversion of extended products from the bead fraction into the supernatant fraction. In these final experiments, about a 120-fold increase in released extension product signal obtained following probing of SKBR3 cells was observed within the supernatant fraction over background signal.

### Example 12: A-ER-v6Tag2 Antibody Conjugate Staining of Cultured SKBR3 vs. MCF7 Cells

To test a different antibody-tag conjugate, the same cell culture samples and capture beads that were prepared and used in α-HER2-v6Tag1 antibody conjugate staining of cultured SKBR3 vs. MCF7 cells, were used for staining with α-ER-v6Tag2 antibody conjugate. Following experiments using capture oligos made of all DNA bases, qPCR results demonstrated about a 0.82-fold increase in detectable released α-ER-v6Tag2 hairpin signal within the bead fraction obtained after staining of MCF7 cells over that of the bead fraction obtained after staining of SKBR3 cells. Within the MCF7 cells, an increase of about 3.5-fold was seen in released α-ER-v6Tag2 hairpin signal on the capture beads versus within the supernatant fraction and about a 10-fold increase over the background signal. Following experiments using capture oligos containing RNA bases with their 5′-ends, qPCR results demonstrated about a 0.97-fold increase in released α-ER-v6Tag2 hairpin signal within the supernatant fraction following staining of MCF7 cells over the signal observed within the supernatant fraction after staining SKBR3 cells. Within the MCF7 cells, about a 5.9-fold increase was seen in released α-ER-v6Tag2 hairpin signal within the supernatant fraction versus that of the bead fraction. These results suggested that the expression levels of the Estrogen Receptor were roughly equivalent between MCF7 and SKBR3 cells, which is not correct based on previous published studies, and was not congruent with in-house experiments whereby these same cells were stained with the non-conjugated ER antibody and visualized using a fluorescently-labeled secondary antibody (ThermoFisher A32732). These initial studies indicated that additional blocking steps may be required for accurate staining and counting of tags released from antibody conjugates that are used for probing proteins of low expression level, as experiments probing HER2 protein levels (this protein is known to be expressed at much higher levels in SKBR3 cells than the ER protein is expressed within the nuclei of MCF7 cells) using the α-HER2-v6Tag1 conjugate did not require additional blocking steps beyond the use of a blocking solution containing SuperBlock™ supplemented with 1 mg/mL BSA and salmon sperm DNA.

### Example 13: Improved Block and Wash Conditions for Antibody Conjugate Staining

Addition of nucleic acid tags to antibodies may lead to non-specific interactions of the conjugated antibodies with other nucleic acids, or with positively charged molecules within the sample. These non-specific interactions may result in the increase of “background” signal in staining experiments. Therefore, a set of experiments were designed to determine more optimal blocking conditions for staining with antibody-tag conjugates used here. In brief, different blocking conditions were tested for non-specific staining by exposing MCF7 cells (low HER2 protein expression level), with the α-HER2-v6Tag1 conjugate, or SKBR3 cells (low to no ER protein expression), with the α-ER-v6Tag2 conjugate. The initial characterization of background signal was conducted with quantitative immunofluorescence (IF) staining and interesting results were further confirmed by qPCR analysis.

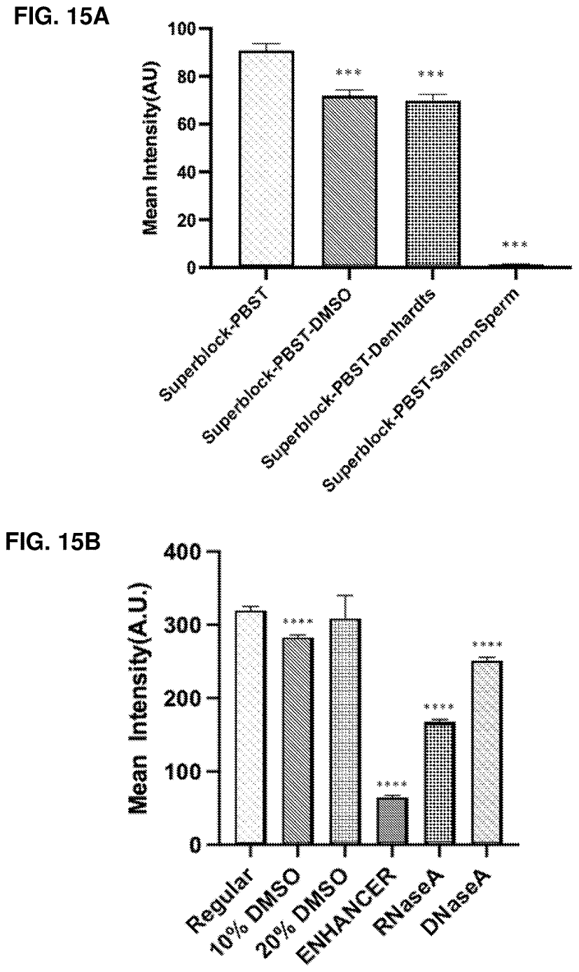

Following staining of MCF7 cells with α-HER2-v6Tag1, the following blocking steps or washing steps were tested: the control staining group was blocked with SuperBlock™ (PBS) blocking buffer (ThermoFisher 37515) and then washed with PBST. From these experiments, a fair amount of background staining was observed in both IF staining and qPCR assays (FIGS. 15A, 15B).

Next, the following conditions were tested and the resulting change in background signal was quantified via IF staining:
Washing with 10% DMSO (1.2-fold decrease in background staining)
Additional blocking with Denhardt's (1.3-fold decrease in background staining)
Additional blocking with Salmon Sperm DNA (67-fold decrease in background staining)

The use of salmon sperm DNA as a blocking agent was further tested in experiments whereby SKBR3 cells (high HER2 protein expression) were stained with α-HER2-v6Tag1, and this blocking agent showed little effect on overall staining signal. In order to confirm the benefits observed from the use of salmon sperm DNA as a blocking agent, MCF7 cells and 1954 cells were blocked and stained with α-HER2-v6Tag1, washed, RNAse H was added, released hairpins were captured, extended on capture oligos bound to capture beads, and samples were quantified by qPCR as described above. From these experiments the following observations were made:

The addition of salmon sperm DNA as a blocking agent when staining MCF7 cells (HER2 negative) with α-HER2-v6Tag1 decreased background staining by about 62-fold via qPCR quantification, which is very similar to the levels observed from IF staining experiments.

The addition of salmon sperm DNA as a blocking agent when staining 1954 cells (HER2 positive) with α-HER2-v6Tag1 decreased positive staining signal by about 1.2-fold via qPCR quantification, further confirming the IF results that showed this blocking agent does not interfere with specific staining by the antibody.

While the addition of salmon sperm DNA was effective in reducing background staining when using the α-HER2-v6Tag1 conjugate, this blocking agent was not effective enough to recover accurate signal from experiments using the α-ER-v6Tag2 conjugate. To further improve upon blocking conditions, IF experiments were performed following staining of SKBR3 cells (ER protein negative) to determine if background staining by the α-ER-v6Tag2 conjugate could be further eliminated. Here, the control condition included salmon sperm DNA within the blocking solution, and the following washing and blocking conditions were tested:
10% DMSO wash—(1.13-fold decrease in background staining)
20% DMSO wash—(1.03-fold decrease in background staining)
Image-iT™ FX signal enhancer—(4.9-fold decrease in background staining)
RNAse A treatment—(1.9-fold decrease in background staining)
DNAse A treatment—(1.27-fold decrease in background staining)

In these experiments, background signal observed within the nucleus and cytoplasm of these cells was calculated independently, and it was clear that the blocking steps were equally effective in both of these compartments of the cell. The above experiment (α-ER-v6Tag2 antibody conjugate staining of cultured SKBR3 vs. MCF7 cells) was repeated with the use of Image-iT™ FX signal enhancer as a blocking agent to test if signal from these experiments could be recovered. The addition of Image-iT™ FX signal enhancer as a blocking agent (in the end-to-end workflow with qPCR read-out) enabled observation of about a 1.8-fold increase in ER expression in MCF7 (ER+) over SKBR3 (ER−) cells. This recovery of ER signal from these experiments although significant, may not completely reflect ER levels in the cells. Therefore, additional optimization of blocking and washing conditions may be beneficial.

### Example 14: Library Construction

To test for sequence bias during hairpin retrieval and PCR amplification, a hairpin library was designed to mimic the structure of the released hairpin species. This library consisted of four random nucleotide positions (Ns) within the loop of the hairpin, representing the position of the various tag barcodes. This library was prepared by IDT via machine mixing of the four different bases at each position. The ordered oligo library was then PAGE-purified in-house using a 10% TBU gel. The library amplification bias test oligo sequence is shown in SEQ ID NO: 37.

This PAGE-purified oligo was then diluted to 0.5 μM in 1× RNAseH buffer. 22.5 μl of this sample was added to 25 μl of Q5® Hot Start High-Fidelity 2× Master Mix and 2.5 μl of each of the following primers:
DRD Forward overhang P5-tag (SEQ ID NO: 20)
DRD Reverse overhang P7-tag (SEQ ID NO: 21)

The primer stock concentration was 1 μM, bringing final concentration in the reaction to 50 nM. The following PCR cycling conditions were utilized: 98° C. (30 sec) (1×), 98° C. (10 sec)->72° C. (15 sec) (25×), 72° C. (4 min) (1×). This reaction produced a single band of ~156 bp which was visualized on a 6% TBE gel stained with SYBR Gold. The PCR reaction (50 μl) was purified with AMPure XP beads at a 1.5× ratio to remove excess (non-extended) primers. The cleaned product from this “first step” of library preparation was eluted from the beads in IBI Scientific PCR grade water (30 μl) and yielded a product of ~5 ng/μl.

For the “second step” of library preparation, 1 μl (5 ng) of the AMPure XP purified product was added to a 50 μl PCR reaction with Q5® Hot Start High-Fidelity 2× Master Mix and 18 cycles of PCR were performed under the same cycling conditions as in the “first step” of library construction, except the following primers were utilized:
P7-G7 index primer (SEQ ID NO: 22)
P5-G7 index primer (SEQ ID NO: 23)

This reaction produced a major band that ran around 225 bp when visualized on a 6% TBE gel stained with SYBR Gold. This 225 bp band was further cleaned up via a two-step size selection with AMPure XP beads (0.6× ratio->1.5×). The cleaned 225 bp band was submitted for Sanger sequencing reactions in both directions with the following primers:
KAPA primer 1 (SEQ ID NO: 24)
KAPA Primer 2 (SEQ ID NO: 25)

### Example 15: Sanger Sequencing of Extended Products

Sanger sequencing was performed with Azenta/GeneWiz for analysis of the PCR-amplified loop library test oligo. The trace files revealed a random, even mix of A, T, C, and G bases at the barcode position “NNNN”. While it is hard to determine if any specific barcode dropout occurred by Sanger sequencing, it does appear that there is possibly a bias for G or C at the first position after the priming site in the tag. To address this issue, one or more G bases may be included immediately downstream of the priming site, for instance in an optimized version of tag v6. NGS analysis of the hairpin loop library test oligo enabled higher resolution of this phenomenon.

### Example 16: NGS Analysis of Extended Products

The same band that was submitted for Sanger sequencing was also submitted for NGS on an Illumina NovaSeq 6000 S4 flow cell (150-paired end). This analysis resulted in a total of Geneious Prime 52,000,000 viable reads. To build a reference genome library for analysis, sequences corresponding to all of the possible 256 (4 base) barcodes were generated in R programming language, and were flanked by 10 nucleotides on either side of the barcode to match that of the expected flanking v6 tag sequence of the template. The paired-end (PE) reads and the “256 Barcode” reference genome were imported into Geneious Prime® 2023.0.4 for trimming and alignment. The paired end reads were trimmed to 10 nucleotides on either side of the barcode location and these reads were aligned with the “Geneious” mapper with 5-iterations of refinement. This alignment demonstrated that 98.6% of the reads mapped to the reference genome library. The percentage of representation of all 256 barcode tags was determined by dividing the number of reads obtained for each barcode sequence by the total number of reads that mapped to the reference genome library. Exhibit B, submitted herewith, provides a table containing the read counts.

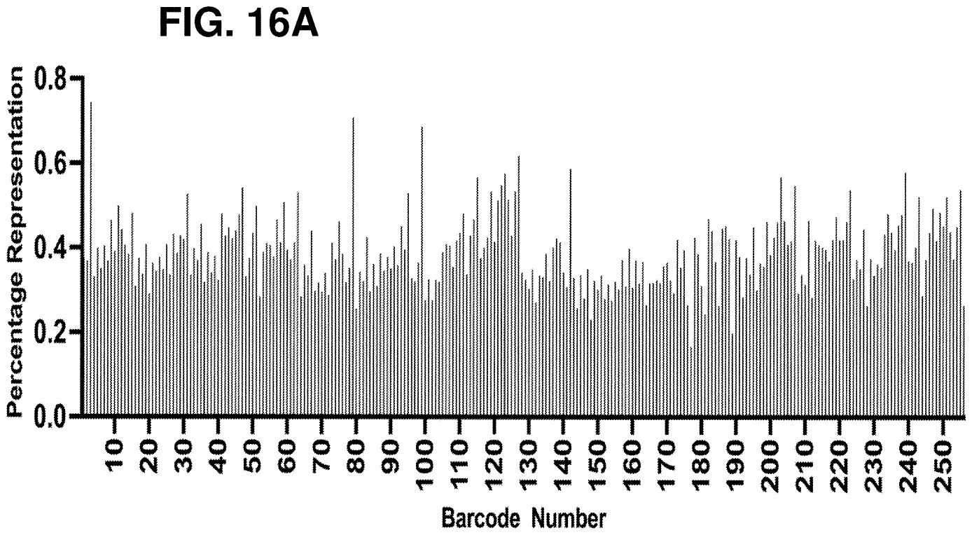

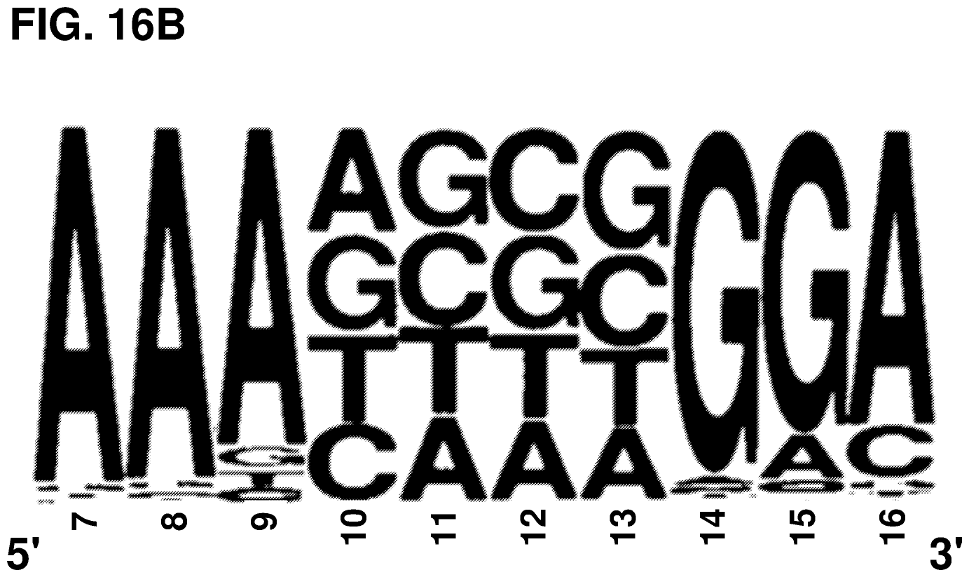

The graph shown in FIG. 16A was generated from 1 million (randomly selected) reads from the 26,000,000 PE reads which were aligned to the reference genome library; the percentage of representation of each barcode was then plotted. This was done 5 times, each time with 1 million reads chosen at random, so as to enable the generation of error calculations. The percentage of reads obtained for each of the 256 barcodes was then plotted using GraphPad Prism.

FIG. 16B is the graphic “sequence logo” (a graphical representation of the patterns within a multiple sequence alignment; see Crooks et al., Genome Res. 14(6):1188-1190, 2004) generated by uploading 10,000 reads (that had been successfully aligned to the reference genome) to WebLogo (online at weblogo.berkeley.edu/logo.cgi).

### Example 17: Capture Surfaces

In some experiments, amino-modified glass slides (AutoMate Scientific Po-104 000 406) or cover glass (AutoMate Scientific 104 000 406) were first modified with 1 mM NHS-LC-Biotin in NHS conjugation buffer to generate biotinylated surfaces. In these experiments, MyOne T1 streptavidin beads resuspended to 2 mg/ml in SA bind buffer were attached to biotinylated surfaces by allowing beads to settle on the surface. Bead-coated surfaces were then washed to remove any unbound beads. In general, this procedure resulted in dense lawns of beads immobilized on the biotinylated surface as determined by bright field imaging at various magnifications. Beads were then coated with a single biotinylated capture oligo type (at 500 nM concentration in SA bind buffer) to generate a dense monolayer of capture beads attached to a surface for various proof of concept experiments. These initial experiments were aimed toward obtaining visual confirmation of successful information (tag) transfer from a sample surface (slide) to the capture array via the isothermal information transfer reaction. To test this, fluorescently-labeled DNA/RNA hybrid hairpin tags modified to contain a 5′ TCO attachment moiety and an internal fluorophore (AF-647 or AF-550) within their loop regions were immobilized on methyltetrazine-modified glass slides. Slides were kept on ice while a solution containing all components required for the isothermal information transfer reaction (200 μM dNTPs, 5000 U/mL Maxima [RNAse]H minus RT (ThermoFisher EP0751) with (Slide 1; experiment 1, see pages 39-41 of Appendix A included in priority U.S. Provisional Application No. 63/487,575, filed on Feb. 28, 2023) or without (Slide 2; experiment 2, see pages 39-41 of Appendix A included in priority U.S. Provisional Application No. 63/487,575, filed on Feb. 28, 2023) inclusion of 500 U/mL RNAse H) was added dropwise to the sample surface. A glass capture array (thin cover glass) consisting of immobilized capture beads coated with a single capture oligo type (containing all DNA bases to enable visualization of the capture step), was then placed face-down to create a “sandwich” to bring capture beads into high proximity with the sample surface in the presence of the two solutions tested. These sandwiched slide-capture arrays were then moved to a heat block (Thermal mixer with Blocks, ThermoFisher 13687711) pre-set to 42° C. and 0 RPM to incubate at the isothermal transfer reaction temperature. Following a 30-minute incubation, capture arrays were removed from slides by adding 1×PBS buffer (ThermoFisher J61196.AP) dropwise to allow the buffer to increase the gap between the capture arrays and the sample slides, enabling their gentle separation. Slides and capture arrays were then washed with additional 1×PBS buffer and imaged using the described imaging parameters. Transfer of tags from a sample surface to the capture array was only observed when performed in the presence of a solution containing RNAse H (Slide 1; experiment 1, see pages 39-41 of Appendix A included in priority U.S. Provisional Application No. 63/487,575, filed on Feb. 28, 2023). It was determined in early attempts to transfer information from a sample surface to this type of capture array (using fluorescently-labeled tag v6 (SEQ ID NO: 7) DNA/RNA hybrid hairpin tags attached to a glass slide in the described isothermal information transfer reaction as described above; data not shown), that capture beads attached to rigid surfaces, such as glass are not optimal for high resolution information transfer. Indeed, large features (of several millimeters in diameter) containing fluorescently-labeled tags could be transferred from a glass slide to capture beads attached to rigid surfaces with high efficiency while maintaining the 2D information contained within the sample surface. However, in early attempts to transfer cellular-level information at sub-cellular resolution (from sample surfaces consisting of fixed Her2+ cells stained with anti-Her2 antibody conjugated with a fluorescently-labeled tag v6; SEQ ID NO: 7) using such a glass capture array were only successful when the two surfaces were placed in very high proximity; this required that the two surfaces be pressed together during the transfer step. This pressure pushed the capture beads into the cells of the sample, and these capture beads were highly fluorescent due to captured tag signal.

It was then postulated that capture beads immobilized within a pliable material such as a gel, would increase the proximity of capture beads with the sample surface without the need to apply as much pressure. Additionally, if the pliable material used for the capture array could be made permeable to macromolecules, it was postulated that enzyme and cofactor components required for (at least initiating) the isothermal information transfer reaction could be added across the permeable gel after the capture array has been placed in high proximity with the sample slide to initiate the transfer reaction. Furthermore, if RNA bases were to be included within the 5′-ends of capture oligos, the extended products of the isothermal information transfer reaction (the complete reaction whereby tags are released from probes bound to a sample surface, captured onto capture oligos, extended to include capture oligo information, and released as products into the surrounding solution) can be performed entirely through the gel matrix. These subsequent experiments testing pliable capture arrays are outlined below.

In other experiments, a pliable, macromolecule-permeable gel was used in place of glass, as a capture array matrix material. MyOne T1 streptavidin beads were first coated with a single, all-DNA capture oligo type as described above, then washed two additional times in 10 mM Tris pH 7.5, and resuspended to 2 mg/ml in 10 mM Tris pH 7.5. This capture bead suspension was then sonicated to reduce clumping of beads and applied directly to amino-modified glass surfaces (AutoMate Scientific Po-104 000 406). Capture beads were allowed to settle down over several hours onto the surface, where they were likely attached primarily through electrostatic interactions between the DNA on the capture beads and the amino groups on the surface. Bead coated surfaces were then washed with 10 mM Tris pH 7.5 to remove any unbound capture beads. In general, this procedure resulted in the generation of dense monolayers of capture beads immobilized on the amino-modified surface as determined by bright field imaging at various magnifications. Bead lawns were generated within particular shapes as defined by 125 μm-thick stickers containing 8 individual holes of 9 mm diameter (GRACE BIO-LABS 6544008) for example. Capture bead surfaces were then placed (with the bead coated surface facing up) on a heat block set to 45° C. As any remaining buffer (10 mM Tris pH 7.5) was allowed to evaporate from bead coated surfaces, molten agarose (high melt temp—catalog number and manufacturer) prepared using 10 mM Tris pH 7.5 at agarose percentages ranging from 0.5-2% was allowed to cool to ~40-45° C., and was slowly applied dropwise directly onto the bead surfaces.

A standard glass slide or cover glass was then used to gently press down on the molten gel to flatten the thickness of the agarose to roughly that of the sticker thickness. After cooling to room temperature, slides or cover glass used for flattening gels were gently removed by sliding-off in a single direction, revealing agarose gel “pads” cast into defined shape containing dense lawns of beads embedded within them. Throughout this exemplified gel casting procedure, electrostatic interactions will maintain capture bead adherence to the amino-modified glass surface if the molten agarose is prepared with a buffer containing little to no salt (10 mM Tris pH 7.5 was used successfully here) and cooled down enough to prevent heat-induced dissociation of beads from the surface (below 50° C. produced successful results here) upon exposure to the molten agarose. Mechanical disruption of bead adherence can also result from adding molten agarose to bead monolayers at too low of a temperature, such that the slide or cover glass used for flattening semi-hardened agarose into thin pads physically disrupts the bead monolayer. Therefore, these experiments were not optimal (in many cases beads were not stably embedded within the gels), but nevertheless arrays of this type were used as a proof of concept for obtaining sub-cellular capture of information from cells (using capture oligos containing all DNA bases). When properly prepared, the pliable bead capture arrays can be peeled away from the glass surface (almost like a thin “skin” of capture beads), and used in capture experiments.

As outlined above, early transfer experiments using the pliable capture arrays were carried out on ice to reduce enzyme activity as the sandwiches were constructed, and then these were moved over to a heat block pre-set to 42° C. to initiate the transfer reaction. To construct the sandwiches on ice, sample slides were placed on a bed of ice in an ice bucket, and a drop of the reaction mixture was placed on the sample slide, far away from the cells bound to the slide. This kept the reaction solution at low temperature prior to dragging the capture bead array “skin” through the solution, for immediate placement (beads down) directly onto the sample slide. A cover glass or glass slide was placed on top of the capture array to apply a small amount of pressure in some cases. In general, pliable capture arrays of this type were very successful in obtaining sub-cellular capture of fluorescently-labeled tags released from probes bound to cells in the presence of isothermal information transfer reaction components (RNAse H, Maxima RT (RNAse H minus), and dNTP cofactors in 1× RNAse H buffer). This was determined using sample surfaces consisting of fixed Her2+ cells stained with anti-Her2 antibody conjugated with a fluorescently-labeled tag v6 (SEQ ID NO: 7). Images of sample slides sandwiched with pliable capture arrays showed that nearly all signal obtained from fluorescent tags had been transferred from probes (bound to cells on sample slides) to capture beads of the capture arrays following a 10-minute incubation at the isothermal reaction temperature (37-42° C.).

In later experiments, RNAse H was removed from the isothermal reaction mixture used during sandwich construction and was included only in a solution applied to the back side of the capture array. In these experiments it was observed that the transfer reaction could be initiated by the addition of the triggering enzyme (RNAse H in this case) via diffusion through the gel. In subsequent experiments, capture oligos containing RNA bases within their 5′-ends were used under the same conditions as the previously described experiments, and a significant enrichment of signal corresponding to full-length product release was detected by Q-PCR only when RNAse H was included in the solution that was applied to the back side of the capture array gel (FIG. 12). These results suggested that not only could one or more of the components required for initiating the isothermal information transfer reaction be applied to the assembled sandwich via diffusion, but full-length products of the reaction could be retrieved through the gel by diffusion as well. This enabled a very simple workflow for the isothermal transfer of information from probed biological samples to a pliable capture array, coupled with retrieval of information from the reaction by aspiration, and no additional user handling.

### Example 18: Conversion of DNA-Encoded Molecule(s) for Isothermal Spatial Encoding

Here, ligation of a T7 (RNA polymerase) promotor-containing adapter directly to the free-end of a dsDNA attached to a probe molecule (biotin) on its opposite, 5′-end (mimicking a probe or library of small molecules) was demonstrated. Following an optional gel purification step, this ligated species or library can be used in binding experiments, for instance upstream of the isothermal spatial encoding workflows described herein.

To demonstrate this, adapter-modified DNA-encoded probe (biotin) was bound to streptavidin beads, unbound probes washed away, and these beads with mixed beads containing capture oligos (CO) in the same reaction tube. In these experiments, the CO did not contain a 3′-inverted T base, but did contain RNA bases in the 5′-end of the CO. A solution containing T7 RNA polymerase and Maxima Reverse Transcriptase was introduced to initiate the one-pot reaction by generating RNA transcripts in vitro, which were then captured onto the capture oligos bound to capture beads, and 3′-ends of COs were extended by the Maxima enzyme to combine information from both species (the transcribed tag and the capture oligo) within a single contiguous DNA sequence.

It was also demonstrated that, by adding a DNA polymerase (Bst 2.0 or 3.0; one which is capable of extending the 3′-end of the captured RNA strand), full-length double-stranded extension products could be generated, and that these products could be released upon RNase H introduction. This alternative assay chemistry can be introduced into the herein described spatial encoding platform, whereby spatially-encoded capture beads are placed over a sample bound to probes. As with other workflows described herein, the resulting contiguous sequence generated with this approach also contains information relating to which probe molecule was present at a given location across a sample, as the information contained within the CO provides spatial coordinates.

### Experimental Methods:

T7p adapter ligations: The ligation of the pre-annealed, dsDNA T7p adaptor (T7p ligation adapter annealed to T7p ligation adapter_rc; SEQ ID NO: 31 and 32, respectively) to the pre-annealed Short tag1 dsDNA oligo containing a contrived A-base overhang (Short Tag1 has A tail annealed to Short Tag1_rc; SEQ ID NO: 29 and 3-, respectively) was carried out in a 200 μl reaction volume containing 1×T4 DNA ligase buffer (NEB M0202S), T4 DNA ligase (NEB M0202S) (final concentration: 30 units/μl), and T4 PNK (NEB M0201S) (final concentration: 0.25 units/μl) using the dsDNA T7p adaptor at a 1 μM concentration, and the dsDNA Short Tag at a 400 nM concentration. Ligation was allowed to proceed overnight at RT. The following day the ligation reaction was EtOH precipitated, resuspended in Ultra-pure H₂O, and cleaned up on Zeba™ Spin Desalting Columns, 7K MWCO (Thermo Fisher 89882) pre-equilibrated with Ultra-pure H₂O. The same procedure was implemented for ligation of the dsDNA T7p adaptor to the Short tag1 dsDNA oligo with no A-base overhang (Short Tag1 need A tail annealed to Short Tag1_rc; SEQ ID NO: 28 and 30, respectively), however, prior to the ligation step, this oligo was subjected to an A-tailing reaction using the NEBNext® Ultra™ II End Repair/dA-Tailing (NEB #E7546S) to add an A-base overhang to this oligo (SEQ ID NO: 29). The A-tailing reaction was cleaned-up using a QIAquick PCR Purification Kit column (Qiagen 28104) and the oligo was eluted in 30 μl of Ultra-pure H₂O prior to ligation with the dsDNA T7p adaptor. Ligation products were analyzed on a 15% TBE-urea gel (Thermo Fisher EC6885BOX), where formation of a 64-base ligation product was observed following both reactions. The ligated constructs were cleaned up via EtOH precipitation, resuspended in Ultra-pure H₂O and cleaned up further using Zeba™ Spin Desalting Columns, 7K MWCO (Thermo Fisher 89882), which had been pre-equilibrated with Ultra-pure H₂O, before utilizing these constructs as templates in downstream in vitro transcription reactions.

In vitro transcription: In vitro transcription reactions were set up to test the various T7 promotor-containing tag constructs (encoding the FL T7p short Tag1) that were generated via the three different approaches described above: (1) The construct that was purchased fully synthesized and biotinylated (FL T7p short Tag1 annealed to FL T7p short Tag1_rc; SEQ ID NO: 26 and 27, respectively); (2) the ligation product of the annealed T7p adaptor (T7p ligation adapter annealed to T7p ligation adapter_rc; SEQ ID NO: 31 and 32, respectively) to the Short tag1 oligo containing a contrived A-base overhang (Short Tag1 has A tail annealed to Short Tag1_rc; SEQ ID NO: 29 and 30, respectively); and (3) the ligation product of the T7p adapter to the Short tag1 oligo with no A-tail (Short Tag1 need A tail annealed to Short Tag1_rc; SEQ ID NO: 28 and 30, respectively). For the tag construct described in (1) above, MyOne T1 streptavidin Dynabeads (ThermoFisher 65601) were used for mimicking a “target” to which the biotin “probe” (attached to this oligo construct) was bound prior to performing standard washes (described elsewhere in this application following streptavidin bead coating with biotinylated oligos) to remove unbound probes. These beads were then equilibrated in 1× transcription buffer prior to subjection to the in vitro transcription reaction. The remaining two species of the tag (2 & 3) were tested for transcript production in solution. All in vitro transcription reactions were set up in the HiScribe® T7 High Yield RNA synthesis Kit (NEB #E2040S) and allowed to incubate at 37° C. for 1 hr. Following this incubation, ⅓ of the volume of each reaction was removed and treated with an RNase Cocktail™ Enzyme Mix (ThermoFisher AM2286) for one hour at 37° C. or TURBO™ DNase (ThermoFisher AM2239). All nuclease reactions were carried out in 1× DNAse I buffer (ThermoFisher AM2239). Gel analysis of transcription reactions was carried out using a 15% TBE-urea gel (Thermo Fisher EC6885BOX), showing that roughly a 10-20-fold amplification of RNA had occurred during the reaction, based on comparison of template band to RNA band (41 bases) intensities. In the case of all three reactions (1-3), a band of the expected size of the RNA product (41 bases) was observed and this band was completely eliminated following treatment with RNAses, while this band was not affected by treatment with DNAse I.

RNA capture and extension: To demonstrate capture of the RNA transcribed from a dsDNA tag that has been modified with the dsDNA T7p adaptor, and to demonstrate templated extension of RNA 3′-ends on a capture oligo (CO), as well as extension of CO 3′-ends, we utilized the same CO that was described previously in this application (containing 5′-RNA bases), but without the inclusion of an inverted T (invT) base at the 3′-end of the CO to enable extension of this CO following capture of the transcribed RNA. The RNA CO without a 3′ invT was built in similar manner to the previous CO via a splint ligation between RNA CO no invT and CO_5_RNA (SEQ ID NO: 35 and 15, respectively), which utilized the CO_Splint (SEQ ID NO: 17) as a template for the ligation. Following ligation, this oligo was PAGE purified and bound to MyOne T1 streptavidin Dynabeads (ThermoFisher 65601) for use in downstream applications.

To test that transcribed RNA could be captured and extended on the CO, transcribed RNA was spiked into solution containing the CO bound to beads at a concentration of 0.25 mg/ml in 1× transcription buffer from the HiScribe® T7 High Yield RNA synthesis Kit (NEB #E2040S) supplemented with 5% DTT, 50 mM KCl, NTPs and dNTPs, as this will be the buffer used to test this alternative one-pot isothermal reaction. This reaction volume was 40 μl, into which 1 μl (8 Units) of Bst 3.0 (NEB M0374S) was added and the reaction was incubated for 1 hr at 37° C. Bst polymerase was used because like Klenow fragment, this polymerase is known to be capable of extending an RNA primer. Following this reaction, beads were pulled down and transferred into 10 mM Tris, heated to 85° C. degrees to de-hybridize the extended RNA, which was retrieved from the supernatant for gel analysis. The supernatant from this reaction was observed on a 15% TBE-urea gel (Thermo Fisher EC6885BOX) whereby a prominent band running at ~80 bp, corresponding to an extension product (of the RNA strand) that was efficiently completed through the DNA-templated region of the CO, but not through the RNA bases templating the 5′-region of the CO. Here, if Bst 3.0 was capable of efficiently extending through the RNA bases, we would expect to see a 96-base product, suggesting Bst 3.0 contains weak reverse transcriptase activity under these conditions. The 80 bp product represents a single stranded product containing RNA at the 5′ end and newly extended DNA bases at the 3′ end; this product will contain primer landing sites for both the CO primer DS 1 and COprimer DS 2. To demonstrate that this observed ~80 bp product truly corresponds to the captured and extended RNA, PCR was performed to create a 79 bp product. No product was generated in the negative controls (Ultra-pure water, non-extended RNA, or CO alone).

To test if the CO could be extended via the captured transcribed RNA, 1 μl of the reverse transcriptase Maxima (ThermoFisher EP0741), which contains RNAse H activity (RNAse H+), was added to the solution containing the CO immobilized on beads, along with RNA that was transcribed from the T7p FL short tag. This step was performed in the same buffer as described above (1× transcription buffer), and the reaction was incubated for 1 hr at 37° C. Beads were then pulled down by magnet, and resuspended in Ultra-pure H₂O. The beads were then heated to 85° C. to break the biotin/streptavidin interaction between the biotin-bound CO and the streptavidin beads. Following heating, beads were pulled down by magnet, and the supernatant was analyzed on a 15% TBE-urea gel (Thermo Fisher EC6885BOX) where a prominent band was observed at ~100 bases, corresponding to the expected size of the fully extended CO (templated by the captured RNA strand). As above, to demonstrate that this observed ~100-base long product truly corresponds to the extended CO, PCR was performed using the CO primer DS 1 and CO primer DS 2 primers to create a 100 bp product, as determined by gel analysis. Although it was possible to amplify the expected product, amplification of a negative control (the CO coated beads+the transcribed RNA, but without Maxima enzyme included) that was subjected to the same heating procedure that was used for disruption of oligo attachment (of the biotin moiety from the streptavidin beads) could not be amplified by PCR using the same primers.

### An Alternative One-Pot Isothermal Reaction:

Streptavidin beads coated with the biotinylated, FL T7p short tag (as described above) were subjected to an in vitro transcription reaction at a final concentration of 0.05 mg/ml, supplemented with 1 μl of T7 RNA polymerase (MO255AVial), Bst 3.0 (8 Units, NEB M0374S) and Maxima reverse transcriptase (200 Units, ThermoFisher EP0741) as well as streptavidin beads coated with the CO (as described above). This experiment was performed to mimic the transfer of information from a surface-bound T7p adaptor-modified tag to another surface containing a CO. Here, the reaction was allowed to proceed at 37° C. for 1 hr. Following the described in vitro transcription-mediated information transfer reaction, RNAse H (5 Units, NEB M0297S) was optionally introduced into the reaction. In both cases the magnetic beads were pulled down onto the magnet at RT and the products in the supernatant were collected. These two supernatants were then run on a 15% TBU gel. In both cases a band at the expected size of the extended CO (~100 bases) was detected, however this band was more prevalent in the reaction that was further treated with RNAse H. In both cases a 79 bp product was amplified via the CO primer DS 1 and CO primer DS 2 primers, indicating that this ~100 base band was indeed the extended CO.

The above results provide data supporting FIG. 7B and FIG. 8.

Restriction Enzyme-Mediated Release of Extension Products from Capture Beads:

To demonstrate restriction enzyme (RE)-mediated release of full-length extension products generated via the alternative one-pot isothermal information transfer reaction described above, a new capture oligo was synthesized (Biotin TEG-DNA CO no InvT; SEQ ID NO: 36) which contains a XhoI restriction site. This oligo was bound to MyOne T1 streptavidin Dynabeads (ThermoFisher 65601) as described above for the CO containing RNA bases in its 5′-end. Following the in vitro transcription-mediated information transfer reaction, half of the reaction was aspirated using a pipette and placed into an identical tube. Both reactions were then subjected to a magnet to pellet beads, the supernatant was removed, beads were resuspended into 1× rCutSmart™ Buffer (NEB B6004S), and 60 Units of XhoI (NEB R0146S) was added to one of the tubes (+XhoI), followed by mixing. Both tubes were allowed to incubate for 15 minutes at 37° C. Following this incubation, both reactions (with and without XhoI) were pulled down onto the magnet and the supernatant from each of the tubes was collected. These supernatants were used in PCR reactions with CO primer DS 1 and CO primer DS 2 (SEQ ID NO: 33 and 34, respectively) followed by gel analysis. A band of the expected size of 59 bp was detected in the reaction containing supernatant from the sample that was treated with XhoI but this band was not present in the reaction containing supernatant from the sample that was not treated with the XhoI enzyme. This result demonstrated that the transferred information (transcribed RNA) was captured by the CO, then extended to include information from both the probe tag as well as the capture oligo (full length extension product), and that this information was then released into solution upon introduction of the XhoI enzyme. The above results provide data supporting FIGS. 9E and 9B.

### List of Oligos Used in Example 18

FL T7p short Tag1: PAGE purified, T7p, Capture region, CO DS primer 1 landing site (SEQ ID NO: 26)
FL T7p short Tag1_Biotin_rc: PAGE purified (SEQ ID NO: 27)
Short Tag1 need A tail: PAGE purified (SEQ ID NO: 28)
Short Tag1 has A tail: PAGE purified_(SEQ ID NO: 29)
Short Tag1_rc: PAGE purified (SEQ ID NO: 30)
T7p ligation adapter: HPLC purified (SEQ ID NO: 31)
T7p ligation adapter_rc: HPLC purified (SEQ ID NO: 32)
CO DS primer 1: standard desalting Tm=47-51 (SEQ ID NO: 33)
CO DS primer 2: standard desalting Tm=47-51 (SEQ ID NO: 34)
RNA CO no invT: Page purified (SEQ ID NO: 35)
CO_5_RNA (SEQ ID NO: 15)
CO 3 invT (SEQ ID NO: 16)
CO_Splint (SEQ ID NO: 17)
Biotin TEG-DNA CO no InvT: HPLC purified, XhoI restriction site (SEQ ID NO: 36)

### SELECT REFERENCES

Fu et al., Cell 185:4621-4633, 2022, doi.org/10.1061/j.cell.2022.10.021
Rodrigues et al., Science 363:1463-1467, 2019
Troll et al., BMC Genomics. 20:1023, 2019, doi.org/10.1186/s12864-019-6355-0
Yang et al., Chembiochem 16(9):1365-1370, 2015
U.S. Pat. No. 10,002,316 “Spatially addressable molecular barcoding:
US 2022/0033802 “Method and apparatus for encoding cellular spatial position information”
US 2020/0190583 “Methods and Compositions for Sequentially Detecting Targets”
WO 2018/087539 “Tagless encoding chemical library”

### Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. A Hairpin Tag nucleic acid molecule comprising, functionally connected in 5′ to 3′ order, parts A-B-C-D-E, wherein:
part A comprises a Cleavable Site comprising either:
(1) a string of RNA bases, or
(2) a string of DNA bases comprising a restriction enzyme (RE) recognition site;
part B comprises a string of DNA bases comprising a Tag PCR Handle;
part C comprises a string of DNA bases comprising a Tag ID Barcode, and which string of DNA bases forms a portion of the loop of the hairpin;
part D comprises a string of DNA bases having the reverse complementary sequence of the Tag PCR Handle, thereby forming a portion of the stem of the hairpin; and
part E comprises a string of DNA bases having the reverse complementary sequence of at least a portion of either (1) the string of RNA bases of part A or (2) the string of DNA bases comprising the RE recognition site, thereby forming a portion of the stem of the hairpin.

2. The Hairpin Tag nucleic acid molecule of claim 1, further comprising an attachment moiety conjugated to the 5′ end of part A.

3. The Hairpin Tag nucleic acid molecule of claim 2, wherein the attachment moiety provides amine-reactive crosslinker activity or thiol-reactive crosslinker activity.

4. The Hairpin Tag nucleic acid molecule of claim 2, further comprising a linker between the attachment moiety and part A.

5. The Hairpin Tag nucleic acid molecule of claim 4, wherein the linker comprises PEG(n), where n=1-20.

6. The Hairpin Tag nucleic acid molecule of claim 1, wherein the Tag ID Barcode is at least 4 bases long.

7. The Hairpin Tag nucleic acid molecule of claim 6, wherein the Tag ID Barcode is 4, 5, 6, 7, 8, or more than 8 bases long.

8. The Hairpin Tag nucleic acid molecule of claim 1, wherein the Tag PCR Handle in part B and the reverse complement thereof in part D are each at least 10 bases long.

9. The Hairpin Tag nucleic acid molecule of claim 8, wherein the Tag PCR Handle in part B and the reverse complement thereof in part D are each 11, 12, 13, 14, 15, 16, 17, 18, 19, 20, or more than 20 bases long.

10. The Hairpin Tag nucleic acid molecule of any one of claims 1-9, which is constructed at least in part using a templated ligation reaction.

11. The Hairpin Tag nucleic acid molecule of any one of claims 1-10, wherein part A comprises the string of DNA bases comprising the RE recognition site, and RE recognition site is at least 4 bases long.

12. The Hairpin Tag nucleic acid molecule of claim 11, wherein the RE recognition site is 4, 5, 6, 7, 8, 9, 10, 11, 12, 13, 14, or longer than 14 bases long.

13. The Hairpin Tag nucleic acid molecule of claim 11, wherein the RE recognition site is a Type IIS Restriction Enzyme recognition site, a site for a RE that produces a 3′ overhang, or both.

14. The Hairpin Tag nucleic acid molecule of any one of claims 1-10 which is a DNA/RNA Hybrid Hairpin Tag nucleic acid molecule, and wherein part A comprises the string of RNA bases, and the string of RNA bases in part A is at least 5 bases long.

15. The DNA/RNA Hybrid Hairpin Tag nucleic acid molecule of claim 14, wherein the string of RNA bases in part A is 5, 6, 7, 8, 9, 10, 11, 12, 13, 14, 15, 16, 17, 18, 19, 20, or longer than 20 bases long.

16. A set of two or more Hairpin Tag nucleic acid molecules of any one of claims 1-13 and/or DNA/RNA Hybrid Hairpin Tag nucleic acid molecules of claim 14 or claim 15, where each of the two or more nucleic acid molecules has a unique Tag ID Barcode sequence.

17. The set of two or more Hairpin Tag nucleic acid molecules and/or DNA/RNA Hybrid Hairpin Tag nucleic acid molecules of claim 16, further comprising at least one Attenuation Tag-like nucleic acid molecule, which Attenuation Tag-like nucleic acid molecule differs from a Hairpin Tag nucleic acid molecule or DNA/RNA Hybrid Hairpin Tag nucleic acid molecule of the set by lack of a functional Cleavable Site.

18. The set of two or more Hairpin Tag nucleic acid molecules and/or DNA/RNA Hybrid Hairpin Tag nucleic acid molecules of claim 17, wherein the Attenuation Tag-like nucleic acid molecule differs from a Hairpin Tag nucleic acid molecule or DNA/RNA Hybrid Hairpin Tag nucleic acid molecules of the set by (1) having DNA bases in place of the RNA bases of part A, or (2) lacks the RE recognition site of part A.

19. A Tagged Probe, comprising a Probe Molecule to which is attached through the attachment moiety to a Hairpin Tag nucleic acid molecule of any one of claims 2-13 or to a DNA/RNA Hybrid Hairpin Tag nucleic acid molecule of claim 14 or claim 15.

20. The Tagged Probe of claim 19, wherein the Probe Molecule comprises an affinity molecule having a binding affinity for a target molecule.

21. The Tagged Probe of claim 20, wherein the affinity molecule comprises an antibody binding domain having affinity for an antigen, and the target molecule comprises the antigen.

22. The Tagged Probe of claim 19, wherein the probe molecule comprises one or more of: an antibody or binding fragment thereof, a nucleic acid, a small molecule, an organic or inorganic chemical, a putative drug target, an identified pharmaceutical drug, or a biological macromolecular complex.

23. The Tagged Probe of claim 19, wherein the probe molecule is one of a set of probe molecules, each of which comprises one of a plurality of members of a library of small molecules, a library of drug targets, a library of biological affinity molecules, a library of natural products, a library of bio-active compounds, a genomic library, a transcriptomic library, a metabolomic library, or a drug screening library.

24. The Tagged Probe of claim 20, wherein the target molecule comprises a biological molecule, an inorganic object, or an addressable feature of an array.

25. The Tagged Probe of claim 24, wherein the target molecule comprises a biological molecule, and the biological molecule comprises one or more of a protein, lipid, carbohydrate, a nucleic acid molecule, or a combination of proteins, lipids, carbohydrates and/or nucleic acid molecules.

26. The Tagged Probe of claim 25 wherein the target molecule is one of a plurality of molecules making up a complex, and the complex is located outside of or within a cell or cells in a tissue sample.

27. The Tagged Probe of any one of claims 19-26, further comprising an amplification sequence comprising a polymerase promoter sequence.

28. The Tagged Probe of claim 27, wherein the amplification sequence comprises a T7 promoter sequence, such as a T7 promoter adaptor.

29. A Released Hairpin Tag nucleic acid molecule derived from a Hairpin Tag of any one of claims 2-13, or from a DNA/RNA Hybrid Hairpin Tag of claim 14 or claim 15, wherein the Released Hairpin Tag has been separated from the attachment moiety by enzymatic action of a Restriction Endonuclease or a RNase H enzyme.

30. A Capture Oligo (CO) nucleic acid molecule comprising, functionally connected in 5′ to 3′ order, parts 1-II-III-IV, wherein:
part I comprises a Cleavable Site comprising either:
(1) a single RNA base or a contiguous string of RNA bases, or
(2) a string of DNA bases comprising a restriction enzyme (RE) recognition site;
part II comprises a string of DNA bases comprising a CO PCR Handle;
part III comprises a string of DNA bases comprising a Spatial Barcode; and
part IV comprises a string of DNA bases comprising a Tag Capture Region.

31. The CO nucleic acid molecule of claim 30, further comprising an attachment moiety conjugated to the 5′ end of part 1.

32. The CO nucleic acid molecule of claim 31, wherein the attachment moiety provides amine-reactive crosslinker activity or thiol-reactive crosslinker activity.

33. The CO nucleic acid molecule of claim 31, further comprising a linker between the attachment moiety and part I.

34. The CO nucleic acid molecule of claim 33, wherein the linker comprises PEG(n), where n=1-20.

35. The CO nucleic acid molecule of claim 30, wherein the Spatial Barcode is at least 4 bases long.

36. The CO nucleic acid molecule of claim 35, wherein the Spatial Barcode is 4, 5, 6, 7, 8, or more than 8 bases long.

37. The CO nucleic acid molecule of claim 30, wherein the CO PCR Handle in part II is at least 5 bases long.

38. The CO nucleic acid molecule of claim 37, wherein the CO PCR Handle in part II is 5, 6, 7, 8, 9, 10, 11, 12, 13, 14, 15, 16, 17, 18, 19, 20, or longer than 20 bases long.

39. The CO nucleic acid molecule of any one of claims 30-38, further comprising a unique molecular identifier (UMI).

40. The CO nucleic acid molecule of any one of claims 30-38, the sequence of which has no more than 2 contiguous bases of internal sequence self-complementarity.

41. The CO nucleic acid molecule of claim 40, the sequence of which has no more than more than 3, no more than 4, no more than 5, no more than 6, no more than 7, no more than 8, no more than 9, no more than 10, no more than 11, or no more than 12 contiguous bases of internal sequence self-complementarity.

42. The CO nucleic acid molecule of any one of claims 30-41, which is constructed at least in part using a templated ligation reaction.

43. The CO nucleic acid molecule of any one of claims 30-42, wherein part I comprises the string of DNA bases comprising the RE recognition site, and RE recognition site is at least 4 bases long.

44. The CO nucleic acid molecule of claim 43, wherein the RE recognition site is 4, 5, 6, 7, 8, 9, 10, 11, 12, 13, 14, or longer than 14 bases long.

45. The CO nucleic acid molecule of claim 43, wherein the RE recognition site is a Type IS Restriction Enzyme recognition site, a site for a RE that leaves a 3′ overhang, or both.

46. The CO nucleic acid molecule of any one of claims 30-42 which is a DNA/RNA chimeric CO nucleic acid molecule, and wherein part I comprises the single RNA base.

47. The CO nucleic acid molecule of any one of claims 30-42 which is a DNA/RNA chimeric CO nucleic acid molecule, and wherein part I comprises the contiguous string of RNA bases.

48. The DNA/RNA chimeric CO nucleic acid molecule of claim 47, wherein the contiguous string of RNA bases in part I is 2, 3, 4, 5, 6, 7, 8, 9, 10, 11, 12, 13, 14, 15, 16, 17, 18, 19, 20 bases long, or longer than 20 bases long.

49. A set of two or more CO nucleic acid molecules of any one of claims 30-45 and/or DNA/RNA chimeric CO nucleic acid molecules of any one of claims 46-48, where each of the two or more nucleic acid molecules has a unique Spatial Barcode sequence.

50. A CO nucleic acid molecule of any one of claims 31-45 or a DNA/RNA chimeric CO nucleic acid molecule of any one of claims 46-48, which is attached to a Capture Feature through the attachment moiety.

51. The CO nucleic acid molecule of claim 50, wherein the Capture Feature is a bead, a chemically-functionalized spot on a glass surface, a defined region of a chemically functionalized and permeable gel, a bead or other inorganic object embedded within or on the surface of a permeable gel, or one of a series of spatially defined objects attached to a gel.

52. The CO nucleic acid molecule of claim 50, wherein the Capture Feature is a spatially addressable feature in an array.

53. The CO nucleic acid molecule of claim 52, wherein the array is a microarray having at least 100 addressable Capture Features.

54. A Spatially Encoded Capture Feature, comprising a Capture Feature to which is attached through the attachment moiety a CO nucleic acid molecule of any one of claims 31-42 or a DNA/RNA chimeric CO nucleic acid molecule of any one of claims 43-48.

55. The Spatially Encoded Capture Feature of claim 54, wherein the Capture Feature comprises a bead or an addressable location on a substantially 2-dimensional surface.

56. The Spatially Encoded Capture Feature of claim 54 or claim 55, which is one Spatially Encoded Capture Feature within an array of at least 100 different Spatially Encoded Capture Features, and wherein the CO nucleic acid molecule on each of the at least 100 different Spatially Encoded Capture Features of the array each comprise a different Spatial Barcode.

57. A Capture Pair, comprising:
a Hairpin Tag nucleic acid molecule of any one of claims 2-13, or a DNA/RNA Hybrid Hairpin Tag nucleic acid molecule of claim 14 or claim 15, or a Released hairpin tag nucleic acid molecule of claim 29; and
a Capture Oligo (CO) nucleic acid molecule of any one of claims 31-42 or a DNA/RNA chimeric CO nucleic acid molecule of any one of claims 43-48, or an Attached CO of any one of claims 50-73,
wherein the sequence of the Hairpin Tag nucleic acid molecule and the sequence of the CO nucleic acid molecule are at least partially complementary, such that when the Hairpin Tag nucleic acid molecule is released from its attachment moiety in proximity to the CO nucleic acid molecule, the released Hairpin Tag nucleic acid molecule is captured by sequence complementarity bonding at the 3′ ends to the CO nucleic acid molecule, such that resultant complex of the released Hairpin Tag nucleic acid molecule and the CO nucleic acid molecule is competent for a downstream extension reaction by a polymerase enzyme.

58. A Spatially Encoding Capture Array, comprising a defined array of spatially-addressed capture features, wherein each capture feature comprises:
a spatially-identifiable feature comprising:
a pre-defined, addressable location on a substantially two-dimensional solid surface; or
a bead or other similar separate, solid capture object; and
attached at each feature, multiple copies of a CO nucleic acid molecule of any one of claims 31-42 or a DNA/RNA chimeric CO nucleic acid molecule of any one of claims 43-48, wherein the CO nucleic acid molecules at each feature have a unique Spatial Barcode sequence compared to the CO nucleic acid molecules at other features in the array.

59. The Spatially Encoding Capture Array of claim 58, wherein one or more CO nucleic acid molecule(s) is applied to spatially-addressed capture feature(s) in the array by:
droplet printing of the CO into the pre-defined, addressable locations on the substantially two-dimensional solid surface, or
attachment of the CO onto beads through the attachment moiety.

60. The Spatially Encoding Capture Array of claim 58 or claim 59, which array comprises beads each of which comprise a Visual Barcode operationally coupled to the COs.

61. The Spatially Encoding Capture Array of claim 60, wherein the Visual Barcode enables beads to be assigned to locations within in the capture array.

62. The Spatially Encoding Capture Array of claim 58, wherein the bead or other similar, separate capture objects are embedded in a biomolecule-permeable matrix.

63. The Spatially Encoding Capture Array of claim 62, wherein:
the capture objects comprise beads;
the biomolecule-permeable matrix comprises a gel; or
both.

64. The Spatially Encoding Capture Array of claim 63, wherein the biomolecule-permeable matrix that comprises the gel is formatted as a pliable sticker.

65. The Spatially Encoding Capture Array of claim 62, wherein the biomolecule-permeable matrix:
is structurally stable at a selected temperature between 4-45° C.;
is permeable to proteins such as functional RNAseH and polymerase;
is permeable to ribonucleoside tri-phosphates (rNTPs) and/or deoxynucleotide triphosphates (dNTPs);
is permeable to Mg²⁺ ions;
is substantially inert to biological molecules; and
is sufficiently flexible to permit application of a relatively thin layer of the matrix directly to a substantially two-dimensional sample or surface.

66. The Spatially Encoding Capture Array of claim 65, which is formulated as a three-dimensional thin-layer gel, the width and length of which are substantially larger than its thickness, and wherein the spatially-identifiable capture features are arranged substantially in a single plane across surface of the gel defined by its the length and width.

67. The Spatially Encoding Capture Array of claim 66, in which at least a first of the spatially-identifiable capture features is immediately adjunct and/or touching a second of the spatially-identifiable capture features.

68. The Spatially Encoding Capture Array of any one of claims 62-67, wherein matrix comprises a hydrogel or polyacrylamide gel.

69. The Spatially Encoding Capture Array of claim 68, wherein the thickness of the matrix or gel is no more than about 2 mm.

70. The Spatially Encoding Capture Array of claim of claim 69, wherein the thickness of the matrix or gel is no more than about 1 mm, no more than 500 μm, no more than 250 μm, no more than 200 μm, no more than 150 μm, no more than 125 μm, no more than 100 μm, or less than 100 μm.

71. The Spatially Encoding Capture Array of claim of claim 70, wherein the thickness of the matrix or gel is 100-200 μm, 100-150 μm, or about 125 μm.

72. The Spatially Encoding Capture Array of any one of claims 58-71, which array is reinforced by an inert mesh or other support structure.

73. The Spatially Encoding Capture Array of any one of claims 58-72, wherein:
the pre-defined, addressable location on a substantially two-dimensional solid surface has a surface area of no more than about 1 μm×1 μm; or
the bead or other similar separate, solid capture object has diameter of no more than about 20 μm.

74. The Spatially Encoding Capture Array of claim 73, wherein the bead or other similar separate, solid capture object has diameter of no more than 18 μm, no more than 15 μm, no more than 12 μm, no more than 10 μm, no more than 8 μm, no more than 5 μm, no more than 3 μm, no more than 1 μm, or about 100 nm.

75. The Spatially Encoding Capture Array of claim 74, wherein the bead or other similar separate, solid capture object has diameter of between 1-3 μm.

76. A semi-ordered Spatially Encoding Capture Array, comprising:
a grid of spatially addressable locations, each of which is labeled with oligonucleotides having a unique X-Y coordinated sequence, which oligonucleotides are applied to the spatially addressable locations by splint ligation of:
a x-coordinate adapter oligonucleotide, which x-coordinate adapter oligonucleotide is used to label all spatially addressable locations within a row of the grid; and
a y-coordinate adapter oligonucleotide, which y-coordinate adapter oligonucleotide is used to label all spatially addressable locations within a column of the grid.

77. The semi-ordered Spatially Encoding Capture Array of claim 76, which is constructed at least in part using a method provided in FIGS. 17A-17C.

78. The semi-ordered Spatially Encoding Capture Array of claim 76, which has a grid-within-a-Grid format as illustrated in FIG. 10.

79. A semi-ordered Spatially Encoding Capture Array, comprising:
an array of uniquely identifiable capture features, which array comprises two or more sub-arrays, each sub-array comprising uniquely identifiable capture features the location of which is specified at least in part by identification of the sub-array within the semi-ordered Spatially Encoding Capture Array.

80. A semi-ordered Spatially Encoding Capture Array, comprising:
a set of two or more sub-arrays each comprising a plurality of capture features, in which each capture feature within each sub-array is attached to a capture oligo comprising a unique Location Tag, the capture features within each sub-array are randomly arranged, and the capture features within each sub-array further comprise a sub-array-identifying oligo tag attached by splint ligation to the capture oligos on each feature in the sub-array.

81. A method for detection and/or quantification and/or localization of targets in a substantially two-dimensional (2D) sample, the method comprising:
contacting a substantially 2D sample with at least one tagged probe to produce a substantially 2D stained sample, which tagged probe comprises:
a Hairpin Tag nucleic acid molecule comprising an attachment moiety and a Tag ID Barcode, or a DNA/RNA Hybrid Hairpin Tag nucleic acid molecule comprising an attachment moiety and a Tag ID Barcode; and
a Probe Molecule attached through the attachment moiety to the Hairpin Tag nucleic acid molecule comprising a Tag ID Barcode, or the DNA/RNA Hybrid Hairpin Tag nucleic acid molecule comprising a Tag ID Barcode;
contacting a surface of the substantially 2D stained sample with a permeable, spatially encoding capture array to form a sample-array sandwich, which spatially encoding capture array comprises:
a plurality of spatially identifiable features; and
attached at each spatially identifiable feature, multiple copies of a Capture Oligo (CO) nucleic acid molecule or a DNA/RNA chimeric CO nucleic acid molecule, wherein the CO nucleic acid molecules at each feature have a unique Spatial Barcode sequence compared to the CO nucleic acid molecules at other features in the array;
placing a flow cell or other solution-containing cover over the sample-array sandwich to form an enclosure containing the sample-array sandwich;
adding to the enclosure a solution comprising reaction components to form a reaction mixture, which components comprise:
a cleavage enzyme selected from a RNAseH or at least one restriction endonuclease (RE);
at least one polymerase;
a mixture of ribonucleoside tri-phosphates (rNTPs) and/or deoxynucleotide triphosphates (dNTPs);
Mg²⁺ ions; and
a buffering agent;
incubating the sample-array sandwich in contact with the reaction mixture at an assay temperature for 30-60 minutes, to form a reaction product mixture;
removing at least a portion of the reaction product mixture from the enclosure; and
analyzing the reaction product mixture to detect and/or quantify and/or define the location of targets in the substantially 2D sample.

82. The method for detection and/or quantification and/or localization of targets in a substantially two-dimensional (2D) sample of claim 81, wherein the enclosure comprises a flow cell.

83. The method of claim 81 or claim 82, which is carried out at a single temperature (isothermally) or within a range of about 5° C. within a single temperature.

84. The method of any one of claims 81-83, wherein the assay temperature has a range of 20-55° C., or 37-42° C.

85. The method of any one of claims 81-84, wherein the method provides location information for more than one target within the substantially 2D sample.

86. The method of any one of claims 81-85, wherein the at least one polymerase provides an enzymatic activity of DNA polymerase, reverse transcriptase, or RNA polymerase.

87. The method of any one of claims 81-86, wherein analyzing the reaction product mixture comprises sequence analysis of a plurality of nucleic acid molecules containing a Spatial Barcode and a Tag ID Barcode.

88. The method of claim 87, wherein analyzing the reaction product mixture comprises next generation sequencing (NGS) of a plurality of nucleic acid molecules containing a Spatial Barcode and a Tag ID Barcode.

89. The method of claim 87 or claim 88, wherein the plurality of nucleic acid molecules containing a Spatial Barcode and a Tag ID Barcode are full extension products released from the spatially identifiable features by cleavage at the Cleavage Site of the CO.

90. The method of claim 89, wherein the cleavage comprises RNAseH enzymatic activity or restriction endonuclease activity.

91. The method of any one of claims 81-90, wherein analyzing comprises assigning a spatial location of at least one nucleic acid molecule containing a Spatial Barcode and a Tag ID Barcode within the substantially 2D sample.

92. A method for isothermal spatial encoding of a biological sample, comprising the method of any one of claims 81-91, wherein the substantially 2D sample is a biological sample.

93. Use of the set of Hairpin Tag nucleic acid molecules of any one of claims 16-18 or the set of Capture Oligo (CO) nucleic acid molecules of claim 49, or both, for transcriptomic analysis of a biological sample.

94. A spatial encoding surface, such as a capture array, for instance a bead-based capture array, substantially as described or illustrated herein.

95. A spatial encoding workflow, comprising:
contacting a hybrid RNA/DNA tag comprising a spatial barcode conjugated to an antibody probe with a substantially two-dimensional (2D) tissue sample, to produce a stained sample;
placing a capture array comprising capture features in contact with the stained sample to produce a sample/array sandwich;
placing a fluid-containment enclosure on top of the sample/array sandwich;
introducing assay solution comprising active RNAseH and active polymerase into the fluid-containment enclosure, thereby bringing the assay solution into contact with the sample/array sandwich;
incubating the sample/array sandwich in the sample solution at a temperature and for a period of time sufficient for the RNAseH activity to at least partially digest the hybrid RNA/DNA tag to produce a cleaved tag and thereby releasing the cleaved tag into the assay solution in proximity to a capture feature;
permitting interaction of the cleaved tag with a capture oligo (CO) on the proximal capture feature to provide a captured cleaved tag;
incubating the sample/array sandwich in the sample solution at a temperature and for a period of time sufficient for the polymerase activity to extend the captured cleaved tag using the CO as template, to produce an extension product;
cleaving the extension product, upon sufficient extension to produce a complementary RNA/DNA region based on the RNA base(s) in the CO, with RNAseH in the sample solution, thereby releasing the full-length extension products;
collecting at least a portion of the full-length, released extension products; and
amplifying and/or sequencing at least one of the full-length, released extension products.

96. A computer readable medium or digital resource, or a digital database, containing spatial location information for features of a spatially encoding array of any one of claims 58-80.

97. The computer readable medium or digital resource, or a digital database, of claim 96, which contains spatial location information for substantially all the features of the spatially encoding array.

98. Use of the computer readable medium or digital resource, or a digital database, of claim 96 or claim 97, to provide a user with location information for one or more targets correlated with the spatial location information of the spatially encoding array.

99. The use of claim 98, wherein the correlation arises through use of the spatially encoding array in a workflow or method as described or illustrated herein.

100. A kit, comprising one or more of:
two or more Hairpin Tag nucleic acid molecules of any one of claims 1-13;
two or more DNA/RNA Hybrid Hairpin Tag nucleic acid molecules of claim 14 or claim 15;
the set of two or more Hairpin Tags of any one of claims 16-18;
a set of two or more Tagged Probes of any one of claims 19-28;
two or more Capture Oligo (CO) nucleic acid molecules of any one of claims 30-46;
two or more DNA/RNA chimeric CO nucleic acid molecules of claim 47 or claim 48;
the set of two or more CO nucleic acid molecules of any one of claims 30-45;
the set of two or more chimeric CO nucleic acid molecules of any of claims 46-48;
two or more attached CO nucleic acid molecules of any one of claims 50-53;
at least one spatially encoding capture array of any one of claims 58-75;
at least one semi-ordered spatially encoding capture array of any one of claims 76-80; or
the spatial encoding surface of claim 94.

101. The kit of claim 100, wherein the spatially encoding capture array, semi-ordered spatially encoding capture array, or spatial encoding surface is in the format of a pliable sticker.

102. The kit of claim 100, further including one or more of:
a container in which is contained functional RNAseH enzyme;
a container in which is contained functional polymerase enzyme;
a container in which is contained functional restriction enzyme;
a container in which is contained one or a mixture of ribonucleoside tri-phosphates (rNTPs) and/or deoxynucleotide triphosphates (dNTPs);
a container in which is contained a solution comprising Mg²⁺ ions; or
a container in which is contained a buffer solution.

103. The kit of claim 101, wherein at least one of the containers contains at least two of the RNAseH enzyme, the polymerase enzyme, the restriction endonuclease enzyme, the rNTPs and/or dNTPs, the Mg²⁺ ions, or the buffer solution.

104. The kit of any one of claims 100-103, further comprising one or more of:
components useful for preparing a sample for analysis using a method provided herein;
a solution-containing cover suitable for placement over a sample-array sandwich on a slide, in order to form a fluid-containing enclosure for the sample-array sandwich; or
a flow cell cover, a glass slide, or other surface suitable for receiving a substantially two-dimensional sample.
